(19) United States
(12) Patent Application Publication
(10) Pub. No.: US 2021/0363152 A1
(43) Pub. Date: Nov. 25, 2021

(54) AMINOAZINE AMIDES

(71) Applicant: Hoffmann-La Roche Inc., Little Falls, NJ (US)

(72) Inventors: Jill Melissa Baccei, Poway, CA (US); Yalda Bravo, San Diego, CA (US); Austin Chih-Yu Chen, San Marcos, CA (US); Ryan Christopher Clark, Santa Barbara, CA (US); Brian Andrew Stearns, Encinitas, CA (US); Yen Pham Hong Truong, San Diego, CA (US)

(21) Appl. No.: 17/311,144

(22) PCT Filed: Dec. 10, 2019
(86) PCT No.: PCT/US2019/065341

§ 371 Date: Jun. 4, 2021

Related U.S. Application Data
(60) Provisional application No. 62/939,853, filed on Nov. 25, 2019.

(30) Foreign Application Priority Data
Dec. 11, 2018 (EP) PCT/EP2018/084377

(51) Int. Cl. C07D 491/107; C07D 403/06; C07D 401/06; C07D 471/10; C07D 495/10; C07D 471/04; C07D 491/048

(52) U.S. Cl. CPC C07D 491/107; C07D 403/06; C07D 401/06; C07D 491/048; C07D 495/10; C07D 471/04; C07D 471/10

(57) ABSTRACT

The disclosure provides novel compounds having the general formula (I) or a pharmaceutically acceptable salt, solvate or salt of the solvate thereof, compositions including the compounds and methods of using the compounds.

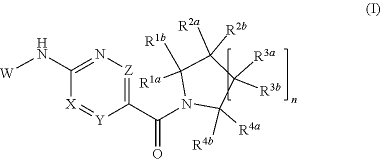

### BACKGROUND

Lyophosphatidic acid (LPA) exerts significant effects on cell growth, motility, survival, and proliferation. LPA levels are regulated, in part, by the glycoprotein autotaxin (ATX), which functions as lysophopholipase D, hydrolyzing lysophosphatidylcholine (LPC) into LPA. Through its modulation of LPA, ATX has been implicated in several physiological processes, including tumor progression and survival, neural development, vascular development, fibrosis, and lymphocyte trafficking. ATX is also thought to be involved in cholestatic and other forms of chronic pruritus and acute and chronic organ transplant rejection. Thus, small molecule inhibitors of ATX will be very valuable, for example, as cancer treatments. In addition they can be used to address organ transplantation or to ameliorate the effects of pruritus.

ATX is an extracellular enzyme that belongs to the ecto nucleotide pyrophosphatse/phosphodiesteraase (ENPP2) family and was first isolated from A2058 melanoma cells. ATX was later found to be glycoprotein lysophospholipase D, the enzyme that catalyzes the production of lysophosphatidic acid (LPA), which in turn, acts through a set of six G-protein coupled receptors (GPCRs) known as LPA₁₋₆, to elicit a wide range of cellular responses including cell proliferation, survival and motility. Increased ATX expression has been detected in chronic liver disease (CLD) and appears to be implicated in cirrhosis and hepatocellular carcinoma (HCC) (See Kaffe et al. Hepatology 2017 April; 65(4):1369-1383), as well as cholestatic pruritus associated with liver disease (See Kremer et al. Gastroenterology, 139 (2010):1008-1018). ATX inhibition and consequent inhibition of downstream LPA signaling has been shown to have therapeutic effect in a mouse model of nonalcoholic steatohepatitis (NASH), and may be a potential therapeutic treatment for fibrotic diseases including multiple fibrotic liver disease and NASH (See Bain et al. J Pharmacol. Exp. Ther. 2017 360(1):1-13). ATX is believed to play a role in inflammation underlying many chronic diseases such as rheumatoid arthritis, multiple sclerosis, atherosclerosis, organ fibrosis including liver and lung fibrosis, hepatitis, asthma, diabetes and obesity (Sevastou et al. Biochimica et Biophysica Acta, 2013, 1831:42-60, Benesch M. et al. FEBS Lett 2014, 588(16): 2712-2727, Park et al. Am. J. Respir. Crit. Care Med., 2013 188:928-940). Increased ATX expression is also found in ulcerative colitis, Crohn's disease and inflammatory bowel disease (See Hozumi et al. Lab. Invest., 2013 93: 508-519). ATX inhibition has also shown efficacy in mouse models of inflammation, inflammatory bowel disease (IBD), multiple sclerosis (MS), and pain (See Thirunavukkarasu K. et al. J Pharmacol Exp Ther. 2016 359(1):207-14 and Saga H., https://doi.org/10.1371/journal/pone/0093230). Upregulated ATX/LPA signaling leads to increased LPA levels and increased LPA receptor production, which is believed to create an environment promoting cancer proliferation, migration, metastasis and cancer therapy resistance. ATX/LPA signaling positively correlates with the invasive and metastatic potential of several cancers including melanoma, breast cancer, ovarian cancer, thyroid cancer, renal cell cancer, lung cancer, neuroblastoma, hepatocellular carcinoma (HCC) and glioblastoma multiforme (See Samadi et al. Biochimie, 2011 93:61-70). ATX is one of many lipid-metabolizing enzymes that play a role in angioproliferative diseases, including those affecting the eye such as age-related macular degeneration (AMD), diabetic retinopathy and retinopathy of prematurity (ROP) (See Stahl et al. Br J Ophthalmol. 2011 95(11): 1496-15010). ATX is also abundant in the human aqueous humor and of possible therapeutic importance in the treatment of ocular hypertension in glaucoma patients (See Iyer et al. https://doi.org/10.1371/journal.pone.0042627). Recent studies also implicate adipose-derived ATX in metabolic disorders including obesity and insulin resistance or diabetes (See D'Souza et al. Endocrinology, 2017 158(4):791-803).

### SUMMARY

The present disclosure relates to certain aminoazine amide compounds, methods of making these compounds, methods of using these compounds, and compositions comprising these compounds.

In some embodiments, the compounds disclosed herein relate to inhibiting ATX, thereby inhibiting LPA production. In some embodiments, the compounds disclosed herein relate to the treatment or prophylaxis in a mammal, wherein the mammal suffers from one or more of a renal condition, a liver condition, an inflammatory condition, a nervous system disorder, a respiratory system disorder, a vascular condition, a cardiovascular condition, a fibrotic disease, cancer, an ocular condition, a metabolic condition, cholestatic pruritis, non-cholestatic pruritus, acute organ transplant rejection, and chronic organ transplant rejection.

For example, The present disclosure provides compounds of Formula (I), or pharmaceutically acceptable salts thereof, compositions comprising compounds of Formula (I), or pharmaceutically acceptable salts thereof and methods of making and using compounds of Formula (I), or pharmaceutically acceptable salts thereof. Compounds of Formula (I), or pharmaceutically acceptable salts thereof, may be used for treating certain diseases, disorders, and conditions, either as mono-therapies or as components of combination therapies. ATX inhibitors such as compounds of Formula (I), or pharmaceutically acceptable salts thereof, may be useful in the treatment or prophylaxis or conditions, diseases, disorders in which the ATX or LPA is involved, such as autoimmune diseases including rheumatoid arthritis and multiple sclerosis, inflammatory diseases including inflammatory bowel disease, ulcerative colitis and Crohn's disease, chronic inflammatory disorders including rheumatoid arthritis (RA), multiple sclerosis (MS), idiopathic pulmonary fibrosis (IPF), hepatitis and atherosclerosis, acute inflammatory disorder such as sepsis, respiratory diseases including asthma, vascular and cardiovascular diseases including atheroscelrosis, fibrotic diseases including fibrosis of the liver, lung, kidney and peritoneum, renal disease, liver diseases including chronic liver disease, cirrhosis, multiple fibrotic liver disease, fatty liver disease and nonalcoholic steatohepatitis (NASH) andcholestatic and other forms of chronic pruritus associated with liver disease, cancer including melanoma, breast cancer, ovarian cancer, thyroid cancer, renal cell cancer, lung cancer, neuroblastoma, hepatocellular carcinoma (HCC) and glioblastoma multiforme, metabolic disorders including diabetes and obesity, diseases of the eye including diabetic retinopathy, ROP. AMD and glaucoma, and acute and chronic organ transplant rejection.

Some embodiments provide compounds of formula (I).

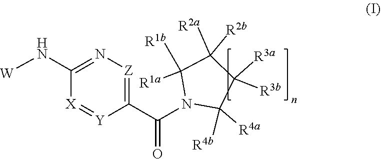

or a pharmaceutically acceptable salt, solvate or salt of the solvate.

In some embodiments W is selected from

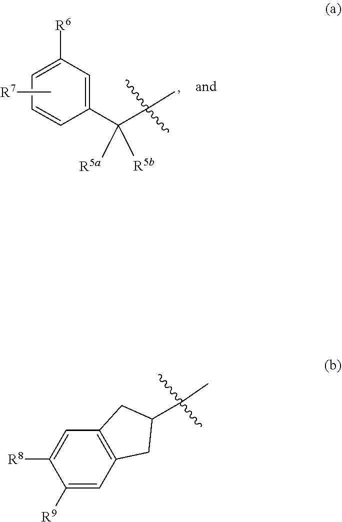

In some embodiments X, Y and Z are each independently selected from N and CH. In some embodiments X is N, and Y and Z are each CH.

In some embodiments n is 0, 1 or 2. In some embodiments n is 0 or 1.

In some embodiments R^{1a} and R^{1b} are each independently selected from (a) hydrogen, (b) C₁₋₆alkyl optionally substituted with OR^{a}, and (c) C₁₋₆haloalkyl. In some embodiments one of R^{1a} and R^{1b} is hydrogen, and the other is selected from (a) hydrogen, (b) C₁₋₄alkyl optionally substituted with OH, and (c) C₁₋₄fluoroalkyl. In some embodiments R^{1a}+R^{1b} together with the carbon atom to which each pair is attached form a (a) 4- to 6-membered carbocycle optionally substituted with one to three groups independently selected from C₁₋₄alkyl, and OH; or (b) 4- to 6-membered heterocycle having 1 heteroatom selected from O, S(O)ₘ, and N—R^{b}.

In some embodiments R^{2a}, R^{2b} R^{3a}, and R^{3b} are each independently selected from (a) hydrogen, (b) C₁₋₆alkyl optionally substituted with OR^{a} or NR^{b}R^{c}, (c) C₁₋₆haloalkyl, (d) halogen, (e) OR^{a}, (f) NR^{b}R^{c}, and (g) S(O)ₘC₁₋₆alkyl. In some embodiments R^{2a}+R^{2b}, together with the carbon atom to which each pair is attached form a (a) 4- to 6-membered carbocycle optionally substituted with one to three groups independently selected from C₁₋₄alkyl, and OH; or (b) 4- to 6-membered heterocycle having 1 heteroatom selected from O, S(O)ₘ, and N—R^{b}. In some embodiments R^{1a}+R^{2a}, and the carbon atoms to which each pair is attached together form a (a) 5-to 6-membered heterocycle having 1 heteroatom selected from O, S(O)ₘ, and N—R^{b}; or (b) 5- to 6-membered heteroaryl.

In some embodiments R^{4a} and R^{4b} are each independently selected from (a) hydrogen, (b) C₁₋₆alkyl optionally substituted with OR^{a}, and (c) C₁₋₆haloalkyl. In some embodiments R^{4a} and R^{4b} are each hydrogen.

In some embodiments R^{5a} and R^{5b} are each independently selected from (a) hydrogen, and (b) C₁₋₆alkyl.

In some embodiments R⁶ is selected from (a) C₁₋₆alkyl, (b) C₁₋₆haloalkyl, (c) (CH₂)ₚC₃₋₆cycloalkyl, (d) OR^{a}, (e) NR^{b}R^{c}, (f) halogen, (g) SF₅, (h) CN, (i) S(O)ₘC₁₋₆alkyl, and (j) C(O)NR^{b}R^{c}. In some embodiments R⁶ is selected from (a) C₁₋₄alkyl, (b) C₁₋₄fluoroalkyl, (c) (CH₂)ₚC₃₋₄cycloalkyl, (d) OC₁₋₄alkyl, (e) OC₁₋₄fluoroalkyl, (f) NR^{b}R^{c}, (g) halogen, (h) SF₅, (i) CN, and (j) S(O)ₘC₁₋₆alkyl. In some embodiments R⁶ is selected from (a) C₁₋₄alkyl, (b) C₁₋₂fluoroalkyl, (c) C₃₋₄cycloalkyl, (d) OC₁₋₄alkyl, and (e) OC₁₋₂fluoroalkyl.

In some embodiments R⁷, R⁸ and R⁹ are each independently selected from (a) hydrogen, and (b) a group under R⁶. In some embodiments R⁷ is selected from (a) C₁₋₄alkyl, (b) C₁₋₄fluoroalkyl, (c) (CH₂)ₚC₃₋₄cycloalkyl, (d) OC₁₋₄alkyl, (e) OC₁₋₄fluoroalkyl, (f) O(CH₂)ₚC₃₋₄cycloalkyl, (g) NR^{b}R^{c}, (h) halogen, and (i) hydrogen. In some embodiments R⁷ is selected from (a) C₁₋₄alkyl, (b) C₁₋₂fluoroalkyl, (c) C₃₋₄cycloalkyl, (d) CH₂C₃₋₄cycloalkyl, (e) OC₁₋₄alkyl, (f) OC₁₋₂fluoroalkyl, (g) OCH₂C₃₋₄cycloalkyl, and (h) azetidinyl.

In some embodiments m is 0, 1 or 2.

In some embodiments p is 0, 1 or 2.

In some embodiments R^{a} is selected from (a) hydrogen, (b) C₁₋₆alkyl, (c) C₁₋₆halolkyl, and (d) (CH₂)ₚC₃₋₆cycloalkyl.

In some embodiments R^{b} and R^{c} are independently selected from (a) hydrogen, (b) —C(O)C₁₋₆alkyl, (c) —SO₂C₁₋₆alkyl, (d) C₁₋₆alkyl optionally substituted with halogen, S(O)ₘC₁₋₆alkyl, (e) C₁₋₆halolkyl, (f) C₃₋₆cycloalkyl, (g) 4- to 6-membered heterocycle, and (h) C(O)OC₁₋₆alkyl. In some embodiments R^{b}, R^{c} and the atom to which they are attached together form a 4- to 6-membered ring optionally containing one additional heteroatom selected from 0, S(O)ₘ, and NH.

In some embodiments the compound of formula (I) is a compound having the formula (Ia)

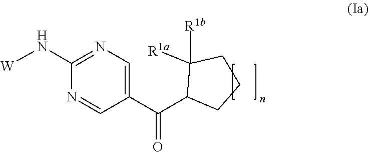

or a pharmaceutically acceptable salt, solvate or salt of the solvate thereof, wherein
W is selected from

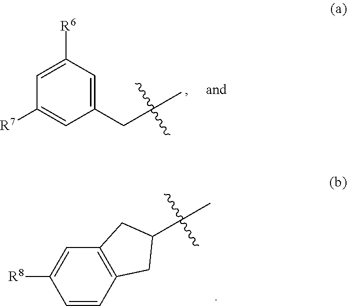

n is 0 or 1;
R⁶ is selected from (a) C₁₋₄alkyl, (b) C₁₋₄fluoroalkyl, (c) (CH₂)ₚC₃₋₄cycloalkyl, (d) OC₁₋₄alkyl, (e) OC₁₋₄fluoroalkyl, (f) NR^{b}R^{c}, (g) halogen, (h) SF₅, (i) CN, and (j) S(O)₂C₁₋₄alkyl;
R⁷ is selected from (a) C₁₋₄alkyl, (b) C₁₋₄fluoroalkyl, (c) (CH₂)ₚC₃₋₄cycloalkyl, (d) OC₁₋₄alkyl, (e) OC₁₋₄fluoroalkyl, (f) O(CH₂)ₚC₃₋₄cycloalkyl, (g) NR^{b}R^{c}, (h) halogen, and (i) hydrogen; and
R⁸ is selected from (a) C₁₋₄alkyl, (b) C₃₋₄cycloalkyl, (c) OC₁₋₄alkyl, (d) halogen, (e) CN, and (f) hydrogen.

Some embodiments provide a pharmaceutical composition comprising a compound of Formula (I), or a pharmaceutically acceptable salt thereof, and a pharmaceutically acceptable carrier.

Some embodiments provide a method for the treatment or prophylaxis of a disease, disorder, or condition selected from the group consisting of: renal disease, liver disease, chronic inflammatory disorder or inflammatory diseases, autoimmune diseases, respiratory disease, vascular and cardiovascular diseases, fibrotic diseases, cancer, ocular disease, metabolic disease, cholestatic and other forms of chronic pruritus and acute and chronic organ transplant rejection, which comprises administering to a patient in need of such treatment a therapeutically effective amount of a compound of formula (I) or a pharmaceutically acceptable salt, solvate or salt of a solvate thereof, or a pharmaceutical composition comprising a compound of formula (I), or a pharmaceutically acceptable salt, solvate or salt of a solvate thereof. Some embodiments provide a method for the treatment of a chronic inflammatory disorder which comprises administering to a patient in need of such treatment, a therapeutically effective amount of a compound of formula (I) or a pharmaceutically acceptable salt, solvate or salt of a solvate thereof, or a pharmaceutical composition comprising a compound of formula (I), or a pharmaceutically acceptable salt, solvate or salt of a solvate thereof. In certain embodiments, the chronic inflammatory disorder is rheumatoid arthritis (RA), multiple sclerosis (MS), idiopathic pulmonary fibrosis (IPF), hepatitis or atherosclerosis.

Some embodiments provide a method for the treatment or prophylaxis of multiple sclerosis which comprises administering to a patient in need of such treatment a therapeutically effective amount of a compound of formula (I) or a pharmaceutically acceptable salt, solvate or salt of a solvate thereof, or a pharmaceutical composition comprising a compound of formula (I), or a pharmaceutically acceptable salt, solvate or salt of a solvate thereof.

Some embodiments provide a method for inhibiting ATX, comprising contacting a cell with a compound of formula (I), or a pharmaceutically acceptable salt, solvate or salt of a solvate thereof. In some embodiments, the cell is a mammalian cell. In some embodiments, the cell is a human cell.

Some embodiments provide a method of decreasing LPA production in a cell, comprising contacting a cell with a compound of formula (I), or a pharmaceutically acceptable salt, solvate or salt of a solvate thereof. In some embodiments, the cell is a mammalian cell. In some embodiments, the cell is a human cell.

### DETAILED DESCRIPTION

Some embodiments provide compounds of formula (I)

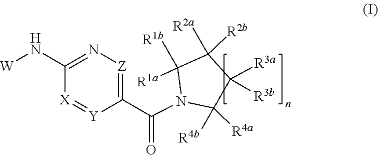

or a pharmaceutically acceptable salt, solvate or salt of the solvate thereof, wherein
W is selected from

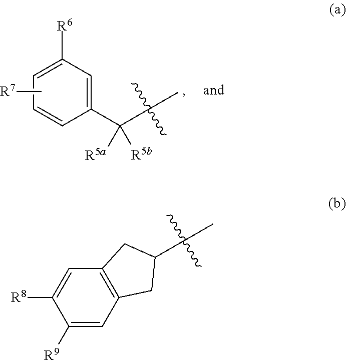

X, Y and Z are each independently selected from N and CH;
n is 0, 1 or 2;
R^{1a} and R^{1b}, R^{4a} and R^{4b} are each independently selected from

(a) hydrogen,

(b) C₁₋₆alkyl optionally substituted with OR^{a}, and

(c) C₁₋₆haloalkyl;

R^{2a}, R^{2b} R^{3a}, and R^{3b} are each independently selected from

(a) hydrogen,

(b) C₁₋₆alkyl optionally substituted with OR^{a} or NR^{b}R^{c},

(c) C₁₋₆haloalkyl,

(d) halogen,

(e) OR^{a},

(f) NR^{b}R^{c}, and

(g) S(O)ₘC₁₋₆alkyl; or

R^{1a}+R^{1b}, or R^{2a}+R^{2b}, or R^{3a}+R^{3b}, together with the carbon atom to which each pair is attached form a

(a) 4- to 6-membered carbocycle optionally substituted with one to three groups independently selected from C₁₋₄alkyl, and OH; or

(b) 4- to 6-membered heterocycle having 1 heteroatom selected from O, S(O)ₘ, and N—R^{b}; or

R^{1a}+R^{2a}, or R^{2a}+R^{3a} and the carbon atoms to which each pair is attached together form a

(a) 5- to 6-membered heterocycle having 1 heteroatom selected from O, S(O)ₘ, and N—R^{b}; or

(b) 5- to 6-membered heteroaryl;

R^{5a} and R^{5b} are each independently selected from

(a) hydrogen, and

(b) C₁₋₆alkyl,

R⁶ is selected from

(a) C₁₋₆alkyl,

(b) C₁₋₆haloalkyl,

(c) (CH₂)ₚC₃₋₆cycloalkyl,

(d) OR^{a},

(e) NR^{b}R^{c},

(f) halogen

(g) SF₅,

(h) CN,

(i) S(O)ₘC₁₋₆alkyl, and

(j) C(O)NR^{b}R^{c},

R⁷, R⁸ and R⁹ are each independently selected from

(a) hydrogen, and

(b) a group under R⁶,

m is 0, 1 or 2;
p is 0, 1 or 2;
R^{a} is selected from

(a) hydrogen,

(b) C₁₋₆alkyl,

(c) C₁₋₆haloalkyl, and

(d) (CH₂)ₚC₃₋₆cycloalkyl,

R^{b} and R^{c} are independently selected from

(a) hydrogen,

(b) —C(O)C₁₋₆alkyl,

(c) —SO₂C₁₋₆alkyl,

(d) C₁₋₆alkyl optionally substituted with S(O)ₘC₁₋₆alkyl,

(e) C₁₋₆halolkyl,

(f) C₃₋₆cycloalkyl,

(g) 4- to 6-membered heterocycle, and

(h) C(O)OC₁₋₆alkyl; or

R^{b}, R^{c} and the atom to which they are attached together form a 4- to 6-membered ring optionally containing one additional heteroatom selected from O, S(O)ₘ, and NH.

In some embodiments X is N, and Y and Z are each CH. In some embodiments Y is N, and X and Z are each CH. In some embodiments Z is N, and X and Y are each CH. In some embodiments X, Y and Z are each CH. In some embodiments n is 0 for the moiety [C(R^{3a})(R^{3b})]ₙ(i.e., the nitrogen containing ring is azetidine). In some embodiments n is 1 for the moiety [C(R^{3a})(R^{3b})]ₙ(i.e., the nitrogen containing ring is pyrrolidine). In some embodiments n is 2 for the moiety [C(R^{3a})(R^{3b})]ₙ(i.e., the nitrogen containing ring is piperidine). In some embodiments R^{2a}, R^{2b}, R^{3a}, R^{3b}, R^{4a}, R^{4b} are each hydrogen. In some embodiments R^{1a}, R^{1b}, R^{3a}, R^{3b}, R^{4a}, R^{4b} are each hydrogen.

In some embodiments, R^{1a} and R^{1b} are each independently selected from (a) hydrogen, (b) C₁₋₆alkyl optionally substituted with OR^{a}, and (c) C₁₋₆haloalkyl; or R^{1a}+R^{1b} together with the carbon atom to which the pair is attached form a (a) 4- to 6-membered carbocycle optionally substituted with one to three groups independently selected from C₁₋₄alkyl, and OH; or (b) 4- to 6-membered heterocycle having 1 heteroatom selected from O, S(O)ₘ, and N—R^{b}; R^{2a}, R^{2b}, R^{3a}, R^{3b}, R^{4a} and R^{4b} are each hydrogen. In some embodiments, one of R^{1a} and R^{1b} is hydrogen, and the other is selected from (a) hydrogen, (b) C₁₋₄alkyl optionally substituted with OH, and (c) C₁₋₄fluoroalkyl. In some embodiments one or both R^{1a} and R^{1b} are hydrogen. In some embodiments one or both R^{1a} and R^{1b} are C₁₋₆alkyl optionally substituted with OR^{a}, for example methyl, ethyl, n-propyl, isopropyl, n-butyl, sec-butyl and t-butyl, each of which may be unsubstituted or substituted with hydroxy, methoxy, ethoxy, n-propoxy, isopropoxy, and the like. In some embodiments one or both R^{1a} and R^{1b} are C₁₋₆haloalkyl for example, —CF₃, —CH₂F, —CF₂H, —CH₂CHF₂, —CH₂CF₃, —CF₂Cl, or —CH(CF₃)₂.

In some embodiments, R^{1a}+R^{1b} together with the carbon atom to which the pair is attached form a: (a) 4- to 6-membered carbocycle optionally substituted with one to three groups independently selected from C₁₋₄alkyl, and OH; or (b) 4- to 6-membered heterocycle having 1 heteroatom selected from O, S(O)ₘ, and N—R^{b}. In some embodiments R^{1a}+R^{1b}, together with the carbon atom to which both are attached form a 4- to 6-membered carbocycle optionally substituted with one to three groups independently selected from C₁₋₄alkyl, and OH, for example cyclopropyl, cyclobutyl, cyclopentyl and cyclohexyl, each of which may be unsubstituted or substituted with one or more groups selected from OH, methyl, ethyl, n-propyl, isopropyl, n-butyl, sec-butyl and t-butyl. In some embodiments R^{1a}+R^{1b}, together with the carbon atom to which both are attached form a 4- to 6-membered heterocycle having 1 heteroatom selected from O, S(O)ₘ, and NR^{b}. In some embodiments the heterocycle is an oxygen containing heterocycle such as oxetane, tetrahydrofuran and tetrahydropyran. In some embodiments the heterocycle is a sulfur-containing heterocycle such as thietane (including oxide and dioxide), tetrahydrothiophene (including oxide and dioxide) and tetrahydrothiopyran (including oxide and dioxide). In some embodiments the heterocycle is a nitrogen-containing heterocycle such as azetidine, pyrrolidine, and piperidine wherein the nitrogen atom of each is unsubstituted or substituted with —C(O)C₁₋₆alkyl (for example, acetyl, n-propanoyl, isopropanoyl, n-butanoyl, sec-butanoyl, and t-butanoyl), —SO₂C₁₋₆alkyl (for example, methanesulfonyl, ethansulfonyl, n-propanesulfonyl, isopropanesulfonyl, n-butanesulfonyl, sec-butanesulfonyl, and t-butanesulfonyl), C₁₋₆alkyl optionally substituted with S(O)ₘC₁₋₆alkyl (for example, methyl, ethyl, n-propyl, isopropyl, n-butyl, sec-butyl, t-butyl, methylsulfonylmethyl, methylsulfonylethyl), C₁₋₆haloalkyl (for example, 2,2-difluoroethyl and 2,2,2-trifluoroethyl), C₃₋₆cycloalkyl (for example, cyclopropyl, cyclobutyl, cyclopentyl and cyclohexyl), 4- to 6-membered heterocycle (for example, oxetanyl, tetrahydrofuranyl, tetrahydropyranyl, azetidinyl, pyrrolidinyl and piperidinyl), or C(O)OC₁₋₆alkyl (for example t-butyloxycarbonyl). In some embodiments of this paragraph, R^{2a}, R^{2b}, R^{3a}, R^{3b}, R^{4a}, R^{4b} are each hydrogen.

In some embodiments, one of R^{2a} and R^{2b} is hydrogen and the other is selected from (a) hydrogen, (b) C₁₋₆alkyl optionally substituted with OR^{a} or NR^{b}R^{c}, (c) C₁₋₆haloalkyl, (d) halogen, (e) OR^{a}, (f) NR^{b}R^{c}, and (g) S(O)ₘC₁₋₆alkyl; or R^{2a}+R^{2b} together with the carbon atom to which the pair is attached form a (a) 4- to 6-membered carbocycle optionally substituted with one to three groups independently selected from C₁₋₄alkyl, and OH; or (b) 4- to 6-membered heterocycle having 1 heteroatom selected from O, S(O)ₘ, and N—R^{b}; and R^{1a}, R^{1b}, R^{3a}, R^{3b}, R^{4a} and R^{4b} are each hydrogen. In some embodiments one or both R^{2a} and R^{2b} are hydrogen. In some embodiments one or both R^{2a} and R^{2b} are C₁₋₆alkyl optionally substituted with OR^{a} or NR^{b}R^{c}, for example methyl, ethyl, n-propyl, isopropyl, n-butyl, sec-butyl and t-butyl, each of which may be unsubstituted or substituted with hydroxy, methoxy, ethoxy, n-propoxy, isopropoxy, amino, methylamino, dimethylamino, and the like. In some embodiments one or both R^{2a} and R^{2b} are C₁₋₆haloalkyl for example, —CF₃, —CH₂F, —CF₂H, —CH₂CHF₂, —CH₂CF₃, —CF₂Cl, or —CH(CF₃)₂. In some embodiments one or both R^{2a} and R^{2b} are halogen, for example, fluoro, chloro, bromo or iodo. In some embodiments one of R^{2a} and R^{2b} is OR^{a} (for example hydroxy, methoxy, ethoxy, n-propoxy, isopropoxy), or NR^{b}R^{c} (for example, amino, methylamino, dimethylamino), or S(O)ₘC₁₋₆alkyl (for example, methylthio, methylsulfinyl, methylsulfonyl).

In some embodiments R^{2a}+R^{2b}, together with the carbon atom to which both are attached form a 4- to 6-membered carbocycle optionally substituted with one to three groups independently selected from C₁₋₄alkyl, and OH, for example cyclopropyl, cyclobutyl, cyclopentyl and cyclohexyl, each of which may be unsubstituted or substituted with one or more groups selected from OH, methyl, ethyl, n-propyl, isopropyl, n-butyl, sec-butyl and t-butyl. In some embodiments R^{2a}+R^{2b}, together with the carbon atom to which both are attached form a 4- to 6-membered heterocycle having 1 heteroatom selected from O, S(O)ₘ, and NR^{b}. In some embodiments the heterocycle is an oxygen containing heterocycle such as oxetane, tetrahydrofuran and tetrahydropyran. In some embodiments the heterocycle is a sulfur-containing heterocycle such as thietane (including oxide and dioxide), tetrahydrothiophene (including oxide and dioxide) and tetrahydrothiopyran (including oxide and dioxide). In some embodiments the heterocycle is a nitrogen-containing heterocycle such as azetidine, pyrrolidine, and piperidine wherein the nitrogen atom of each is unsubstituted or substituted with —C(O)C₁₋₆alkyl (for example, acetyl, n-propanoyl, isopropanoyl, n-butanoyl, sec-butanoyl, and t-butanoyl), —SO₂C₁₋₆alkyl (for example, methanesulfonyl, ethansulfonyl, n-propanesulfonyl, isopropanesulfonyl, n-butanesulfonyl, sec-butanesulfonyl, and t-butanesulfonyl), C₁₋₆alkyl optionally substituted with S(O)ₘC₁₋₆alkyl (for example, methyl, ethyl, n-propyl, isopropyl, n-butyl, sec-butyl, t-butyl, methylsulfonylmethyl, methylsulfonylethyl), C₁₋₆haloalkyl (for example, 2,2-difluoroethyl and 2,2,2-trifluoroethyl), C₃-6cycloalkyl (for example, cyclopropyl, cyclobutyl, cyclopentyl and cyclohexyl), 4- to 6-membered heterocycle (for example, oxetanyl, tetrahydrofuranyl, tetrahydropyranyl, azetidinyl, pyrrolidinyl and piperidinyl), or C(O)OC₁₋₆alkyl (for example t-butyloxycarbonyl). In some embodiments of this paragraph, R^{1a}, R^{1b}, R^{3a}, R^{3b}, R^{4a}, R^{4b} are each hydrogen.

In some embodiments each R^{3a} and R^{3b} is hydrogen.

In some embodiments one or both R^{4a} and R^{4b} are hydrogen. In some embodiments one or both R^{4a} and R^{4b} are C₁₋₆alkyl (for example methyl, ethyl, n-propyl, isopropyl, n-butyl, sec-butyl, t-butyl).

In some embodiments, n is 0 or 1. In some embodiments n is 0. In some embodiments n is 1. In some embodiments n is 2.

In some embodiments, W is selected from

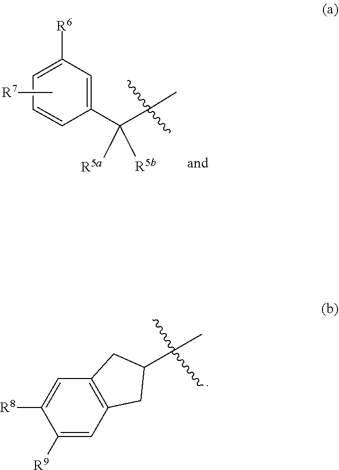

In some embodiments W is

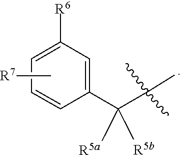

In some embodiments W is

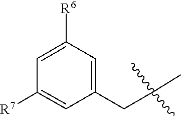

In some embodiments W is

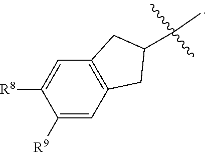

In some embodiments W is

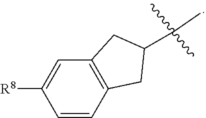

In some embodiments, W is selected from

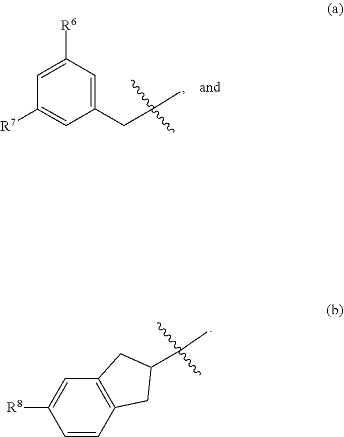

In some embodiments one or both R^{5a} and R^{5b} are hydrogen. In some embodiments one or both R^{5a} and R^{5b} are C₁₋₆alkyl (for example methyl, ethyl, n-propyl, isopropyl, n-butyl, sec-butyl, t-butyl).

In some embodiments R⁶ is —O—C₁₋₆alkyl, for example methoxy, ethoxy, n-propyloxy, isopropoxy, n-butoxy, sec-butoxy, t-butoxy; in some embodiments R⁶ is —O—C₁₋₄alkyl. In some embodiments R⁶ is —O—C₁₋₆halolkyl, for example fluoromethoxy, difluoromethoxy, trifluoromethoxy, 2-fluoroethoxy, 2,2-difluoroethoxy, 2,2,2-trifluoroethoxy; in some embodiments R⁶ is —O—C₁₋₄fluoroalkyl or —O—C₁₋₂fluoroalkyl. In some embodiments R⁶ is —O—(CH₂)ₚC₃₋₆cycloalkyl, for example cyclobutyloxy, cyclopropyloxy, cyclopropylmethoxy, cyclobutylmethoxy, cyclopropylethoxy, cyclobutylethoxy. In some embodiments R⁶ is C₁₋₆alkyl, for example methyl, ethyl, n-propyl, isopropyl, n-butyl, sec-butyl, t-butyl; in some embodiments R⁶ is —C₁₋₄alkyl. In some embodiments R⁶ is C₁₋₆halolkyl, for example fluoromethyl, difluoromethyl, trifluoromethyl, 2-fluoroethyl, 2,2-difluoroethyl, 2,2,2-trifluoroethyl; in some embodiments R⁶ is C₁₋₄fluoroalkyl or C₁₋₂fluoroalkyl. In some embodiments R⁶ is halogen, for example fluoro, chloro, bromo and iodo. In some embodiments R⁶ is (CH₂)ₚC₃₋₆cycloalkyl, for example cyclobutyl, cyclopropyl, cyclopropylmethyl, cyclobutylmethyl, cyclopropylethyl, cyclobutylethyl. In some embodiments R⁶ is NR^{b}R^{c}, for example amino, methylamino, dimethylamino, azetidine, pyrrolidine and piperidine. In some embodiments R⁶ is CN. In some embodiments R⁶ is SF₅. In some embodiments R⁶ is S(O)ₘC₁₋₆alkyl, for example methylthio, methylsulfinyl, methylsulfonyl. In some embodiments R⁶ is C(O)NR^{b}R^{c}, for example, carboxamide, N-methylcarboxamide, N,N-dimethylcarboxamide, 1-azetidinylcarbonyl.

In some embodiments R⁷ is hydrogen. In some embodiments R⁷ is —O—C₁₋₆alkyl, for example methoxy, ethoxy, n-propyloxy, isopropoxy, n-butoxy, sec-butoxy, t-butoxy; in some embodiments R⁷ is —O—C₁₋₄alkyl. In some embodiments R⁷ is —O—C₁₋₆halolkyl, for example fluoromethoxy, difluoromethoxy, trifluoromethoxy, 2-fluoroethoxy, 2,2-difluoroethoxy, 2,2,2-trifluoroethoxy; in some embodiments R⁷ is —O—C₁₋₄fluoroalkyl or —O—C₁₋₂fluoroalkyl. In some embodiments R⁷ is —O—(CH₂)ₚC₃₋₆cycloalkyl, for example cyclobutyloxy, cyclopropyloxy, cyclopropylmethoxy, cyclobutylmethoxy, cyclopropylethoxy, cyclobutylethoxy. In some embodiments R⁷ is C₁₋₆alkyl, for example methyl, ethyl, n-propyl, isopropyl, n-butyl, sec-butyl, t-butyl; in some embodiments R⁷ is —C₁₋₄alkyl. In some embodiments R⁷ is C₁₋₆halolkyl, for example fluoromethyl, difluoromethyl, trifluoromethyl, 2-fluoroethyl, 2,2-difluoroethyl, 2,2,2-trifluoroethyl; in some embodiments R⁷ is C₁₋₄fluoroalkyl or C₁₋₂fluoroalkyl. In some embodiments R⁷ is halogen, for example fluoro, chloro, bromo and iodo. In some embodiments R⁷ is (CH₂)ₚC₃₋₆cycloalkyl, for example cyclobutyl, cyclopropyl, cyclopropylmethyl, cyclobutylmethyl, cyclopropylethyl, cyclobutylethyl. In some embodiments R⁷ is NR^{b}R^{c}, for example amino, methylamino, dimethylamino, azetidine, pyrrolidine and piperidine. In some embodiments R⁷ is CN. In some embodiments R⁷ is SF₅. In some embodiments R⁷ is S(O)ₘC₁₋₆alkyl, for example methylthio, methylsulfinyl, methylsulfonyl. In some embodiments R⁷ is C(O)NR^{b}R^{c}, for example, carboxamide, N-methylcarboxamide, N,N-dimethylcarboxamide, 1-azetidinylcarbonyl.

In some embodiments R⁸ is hydrogen. In some embodiments R⁸ is —O—C₁₋₆alkyl, for example methoxy, ethoxy, n-propyloxy, isopropoxy, n-butoxy, sec-butoxy, t-butoxy; in some embodiments R⁸ is —O—C₁₋₄alkyl. In some embodiments R⁸ is —O—C₁₋₆halolkyl, for example fluoromethoxy, difluoromethoxy, trifluoromethoxy, 2-fluoroethoxy, 2,2-difluoroethoxy, 2,2,2-trifluoroethoxy; in some embodiments R⁸ is —O—C₁₋₄fluoroalkyl or —O—C₁₋₂fluoroalkyl. In some embodiments R⁸ is —O—(CH₂)ₚC₃₋₆cycloalkyl, for example cyclobutyloxy, cyclopropyloxy, cyclopropylmethoxy, cyclobutylmethoxy, cyclopropylethoxy, cyclobutylethoxy. In some embodiments R⁸ is C₁₋₆alkyl, for example methyl, ethyl, n-propyl, isopropyl, n-butyl, sec-butyl, t-butyl; in some embodiments R⁸ is —C₁₋₄alkyl. In some embodiments R⁸ is C₁₋₆halolkyl, for example fluoromethyl, difluoromethyl, trifluoromethyl, 2-fluoroethyl, 2,2-difluoroethyl, 2,2,2-trifluoroethyl; in some embodiments R⁸ is C₁₋₄fluoroalkyl or C₁₋₂fluoroalkyl. In some embodiments R⁸ is halogen, for example fluoro, chloro, bromo and iodo. In some embodiments R⁸ is (CH₂)ₚC₃₋₆cycloalkyl, for example cyclobutyl, cyclopropyl, cyclopropylmethyl, cyclobutylmethyl, cyclopropylethyl, cyclobutylethyl. In some embodiments R⁸ is NR^{b}R^{c}, for example amino, methylamino, dimethylamino, azetidine, pyrrolidine and piperidine. In some embodiments R⁸ is CN. In some embodiments R⁸ is SF₅. In some embodiments R⁸ is S(O)ₘC₁₋₆alkyl, for example methylthio, methylsulfinyl, methylsulfonyl. In some embodiments R⁸ is C(O)NR^{b}R^{c}, for example, carboxamide, N-methylcarboxamide, N,N-dimethylcarboxamide, 1-azetidinylcarbonyl.

In some embodiments R⁹ is hydrogen. In some embodiments R⁹ is —O—C₁₋₆alkyl, for example methoxy, ethoxy, n-propyloxy, isopropoxy, n-butoxy, sec-butoxy, t-butoxy; in some embodiments R⁹ is —O—C₁₋₄alkyl. In some embodiments R⁹ is —O—C₁₋₆halolkyl, for example fluoromethoxy, difluoromethoxy, trifluoromethoxy, 2-fluoroethoxy, 2,2-difluoroethoxy, 2,2,2-trifluoroethoxy; in some embodiments R⁹ is —O—C₁₋₄fluoroalkyl or —O—C₁₋₂fluoroalkyl. In some embodiments R⁹ is —O—(CH₂)ₚC₃₋₆cycloalkyl, for example cyclobutyloxy, cyclopropyloxy, cyclopropylmethoxy, cyclobutylmethoxy, cyclopropylethoxy, cyclobutylethoxy. In some embodiments R⁹ is C₁₋₆alkyl, for example methyl, ethyl, n-propyl, isopropyl, n-butyl, sec-butyl, t-butyl; in some embodiments R⁹ is —C₁₋₄alkyl. In some embodiments R⁹ is C₁₋₆halolkyl, for example fluoromethyl, difluoromethyl, trifluoromethyl, 2-fluoroethyl, 2,2-difluoroethyl, 2,2,2-trifluoroethyl; in some embodiments R⁹ is C₁₋₄fluoroalkyl or C₁₋₂fluoroalkyl. In some embodiments R⁹ is halogen, for example fluoro, chloro, bromo and iodo. In some embodiments R⁹ is (CH₂)ₚC₃₋₆cycloalkyl, for example cyclobutyl, cyclopropyl, cyclopropylmethyl, cyclobutylmethyl, cyclopropylethyl, cyclobutylethyl. In some embodiments R⁹ is NR^{b}R^{c}, for example amino, methylamino, dimethylamino, azetidine, pyrrolidine and piperidine. In some embodiments R⁹ is CN. In some embodiments R⁹ is SF₅. In some embodiments R⁹ is S(O)ₘC₁₋₆alkyl, for example methylthio, methylsulfinyl, methylsulfonyl. In some embodiments R⁹ is C(O)NR^{b}R^{c}, for example, carboxamide, N-methylcarboxamide, N,N-dimethylcarboxamide, 1-azetidinylcarbonyl.

In some embodiments, provided are compounds of formula (I) having the formula (Ia)

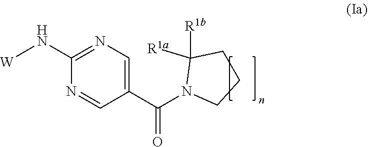

wherein the variables are as described elsewhere herein. In certain embodiments, W is selected from

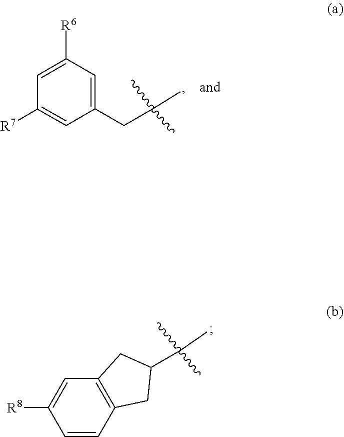

n is 0 or 1; R⁶ is selected from (a) C₁₋₄alkyl, (b) C₁₋₄fluoroalkyl, (c) (CH₂)ₚC₃₋₄cycloalkyl, (d) OC₁₋₄alkyl, (e) OC₁₋₄fluoroalkyl, (f) NR^{b}R^{c}, (g) halogen, (h) SF₅, (i) CN, and (j) S(O)₂C₁₋₄alkyl; R⁷ is selected from (a) C₁₋₄alkyl, (b) C₁₋₄fluoroalkyl, (c) (CH₂)ₚC₃₋₄cycloalkyl, (d) OC₁₋₄alkyl, (e) OC₁₋₄fluoroalkyl, (f) O(CH₂)ₚC₃₋₄cycloalkyl, (g) NR^{b}R^{c}, (h) halogen, and (i) hydrogen; R⁸ is selected from (a) C₁₋₄alkyl, (b) C₃₋₄cycloalkyl, (c) OC₁₋₄alkyl, (d) halogen, (e) CN, and (f) hydrogen.

In some embodiments the compound of formula (I) is selected from the group consisting of:
(2-((3-isopropyl-5-(trifluoromethoxy)benzyl)amino)pyrimidin-5-yl)(6-oxa-1-azaspiro[3.3]heptan-1-yl)methanone;
6-oxa-1-azaspiro[3.3]heptan-1-yl(2-((3-(trifluoromethoxy)benzyl)amino)pyrimidin-5-yl)-methanone;
2-oxa-6-azaspiro[3.3]heptan-6-yl(2-((3-(trifluoromethoxy)benzyl)amino)pyrimidin-5-yl)-methanone;
(2-((3-(tert-butyl)-5-(trifluoromethoxy)benzyl)amino)pyrimidin-5-yl)(6-oxa-1-azaspiro[3.3]heptan-1-yl)methanone;
(2-((3-(tert-butyl)-5-(trifluoromethoxy)benzyl)amino)pyrimidin-5-yl)(2-methylazetidin-1-yl)methanone;
(2-((3-isopropyl-5-(trifluoromethoxy)benzyl)amino)pyrimidin-5-yl)(2-methylazetidin-1-yl)methanone;
(2-((2,3-dihydro-1H-inden-2-yl)amino)pyrimidin-5-yl)(6-oxa-1-azaspiro[3.3]heptan-1-yl)methanone;
azetidin-1-yl(2-((2,3-dihydro-1H-inden-2-yl)amino)pyrimidin-5-yl)methanone;
(2-((3,5-bis(trifluoromethyl)benzyl)amino)pyrimidin-5-yl)(2-methylazetidin-1-yl)-methanone;
(2-((3,5-dichlorobenzyl)amino)pyrimidin-5-yl)(2-methylazetidin-1-yl)methanone;
(2-((3,5-dimethoxybenzyl)amino)pyrimidin-5-yl)(2-methylazetidin-1-yl)methanone;
(2-((3-methyl-5-(trifluoromethoxy)benzyl)amino)pyrimidin-5-yl)(2-methylazetidin-1-yl)-methanone;
6-oxa-1-azaspiro[3.3]heptan-1-yl(2-((1-(3-(trifluoromethoxy)phenyl)ethyl)amino)pyrimidin-5-yl)methanone;
3-(((5-(6-oxa-1-azaspiro[3.3]heptane-1-carbonyl)pyrimidin-2-yl)amino)methyl)-benzonitrile;
(2-((3-chlorobenzyl)amino)pyrimidin-5-yl)(6-oxa-1-azaspiro[3.3]heptan-1-yl)methanone;
(2-((3-(methylsulfonyl)benzyl)amino)pyrimidin-5-yl)(6-oxa-1-azaspiro[3.3]heptan-1-yl)-methanone;
(2-((3-(difluoromethoxy)benzyl)amino)pyrimidin-5-yl)(6-oxa-1-azaspiro[3.3]heptan-1-yl)methanone;
(2-((3-(difluoromethyl)benzyl)amino)pyrimidin-5-yl)(6-oxa-1-azaspiro[3.3]heptan-1-yl)-methanone;
(2-((3-isopropyl-5-(pentafluorothio)benzyl)amino)pyrimidin-5-yl)(6-oxa-1-azaspiro[3.3]-heptan-1-yl)methanone;
6-oxa-1-azaspiro[3.3]heptan-1-yl(2-((3-(trifluoromethyl)benzyl)amino)pyrimidin-5-yl)-methanone;
(2-((3-isopropyl-5-(trifluoromethoxy)benzyl)amino)pyrimidin-5-yl)(pyrrolidin-1-yl)-methanone;
(2-((3-isopropyl-5-(trifluoromethoxy)benzyl)amino)pyrimidin-5-yl)(piperidin-1-yl)methanone;
(2-((3-cyclobutyl-5-(trifluoromethoxy)benzyl)amino)pyrimidin-5-yl)(6-oxa-1-azaspiro[3.3]heptan-1-yl)methanone;
6-oxa-1-azaspiro[3.3]heptan-1-yl(2-((3-(pentafluorothio)benzyl)amino)pyrimidin-5-yl)-methanone;
(2-((3-bromo-5-(trifluoromethoxy)benzyl)amino)pyrimidin-5-yl)(6-oxa-1-azaspiro[3.3]-heptan-1-yl)methanone;
(2-((3-fluoro-5-(trifluoromethoxy)benzyl)amino)pyrimidin-5-yl)(6-oxa-1-azaspiro[3.3]-heptan-1-yl)methanone;
(2-((3-(cyclopropylmethoxy)-5-(trifluoromethoxy)benzyl)amino)pyrimidin-5-yl)(6-oxa-1-azaspiro[3.3]heptan-1-yl)methanone;
(2-((3-isopropoxy-5-(trifluoromethoxy)benzyl)amino)pyrimidin-5-yl)(6-oxa-1-azaspiro[3.3]heptan-1-yl)methanone;
(2-((3-(cyclopropylmethyl)-5-(trifluoromethoxy)benzyl)amino)pyrimidin-5-yl)(6-oxa-1-azaspiro[3.3]heptan-1-yl)methanone;
(2-((3-methoxy-5-(trifluoromethoxy)benzyl)amino)pyrimidin-5-yl)(6-oxa-1-azaspiro[3.3]heptan-1-yl)methanone;
(2-((3-ethoxy-5-(trifluoromethoxy)benzyl)amino)pyrimidin-5-yl)(6-oxa-1-azaspiro[3.3]-heptan-1-yl)methanone;
(2-((5-methoxy-2,3-dihydro-1H-inden-2-yl)amino)pyrimidin-5-yl)(6-oxa-1-azaspiro[3.3]-heptan-1-yl)methanone;
(2-((5-chloro-2,3-dihydro-1H-inden-2-yl)amino)pyrimidin-5-yl)(6-oxa-1-azaspiro[3.3]heptan-1-yl)methanone;
2-((5-(6-oxa-1-azaspiro[3.3]heptane-1-carbonyl)pyrimidin-2-yl)amino)-2,3-dihydro-1H-indene-5-carbonitrile;
6-oxa-1-azaspiro[3.3]heptan-1-yl(2-((3-(2,2,2-trifluoroethoxy)-5-(trifluoromethoxy)-benzyl)amino)pyrimidin-5-yl)methanone;
(2-((3-cyclopropyl-5-(trifluoromethyl)benzyl)amino)pyrimidin-5-yl)(6-oxa-1-azaspiro[3.3]heptan-1-yl)methanone;
(2-((3,5-dicyclopropylbenzyl)amino)pyrimidin-5-yl)(6-oxa-1-azaspiro[3.3]heptan-1-yl)-methanone;
(2-((3-cyclopropyl-5-isopropoxybenzyl)amino)pyrimidin-5-yl)(6-oxa-1-azaspiro[3.3]-heptan-1-yl)methanone;
(2-((3-isopropoxy-5-propylbenzyl)amino)pyrimidin-5-yl)(6-oxa-1-azaspiro[3.3]heptan-1-yl)methanone;
(2-((5-chloro-2,3-dihydro-1H-inden-2-yl)amino)pyrimidin-5-yl)(7-methyl-1,7-diazaspiro[3.5]nonan-1-yl)methanone;
(2-((5-chloro-2,3-dihydro-1H-inden-2-yl)amino)pyrimidin-5-yl)(2,2-dimethylazetidin-1-yl)methanone;
(2-((5-fluoro-2,3-dihydro-1H-inden-2-yl)amino)pyrimidin-5-yl)(6-oxa-1-azaspiro[3.3]-heptan-1-yl)methanone;
(2-((5-bromo-2,3-dihydro-1H-inden-2-yl)amino)pyrimidin-5-yl)(6-oxa-1-azaspiro[3.3]heptan-1-yl)methanone;
(2-((5-chloro-2,3-dihydro-TH-inden-2-yl)amino)pyrimidin-5-yl)(2-methylazetidin-1-yl)-methanone;
(2-((5-chloro-2,3-dihydro-1H-inden-2-yl)amino)pyrimidin-5-yl)(2-(difluoromethyl)-azetidin-1-yl)methanone;
(2-((5-chloro-2,3-dihydro-1H-inden-2-yl)amino)pyrimidin-5-yl)(2-oxa-5-azaspiro[3.4]octan-5-yl)methanone;
(2-((5-chloro-2,3-dihydro-TH-inden-2-yl)amino)pyrimidin-5-yl)(7-oxa-1-azaspiro[3.5]nonan-1-yl)methanone;
(2-((5-chloro-2,3-dihydro-TH-inden-2-yl)amino)pyrimidin-5-yl)(6-oxa-1-azaspiro[3.5]nonan-1-yl)methanone;
(2-((5-chloro-2,3-dihydro-TH-inden-2-yl)amino)pyrimidin-5-yl)(6,6-dioxido-6-thia-1-azaspiro[3.3]heptan-1-yl)methanone;
(2-((3,5-dichlorobenzyl)amino)pyrimidin-5-yl)(6-oxa-1-azaspiro[3.3]heptan-1-yl)-methanone;
(2-((3,4-dichlorobenzyl)amino)pyrimidin-5-yl)(6-oxa-1-azaspiro[3.3]heptan-1-yl)-methanone;
(2-((3,5-diisopropoxybenzyl)amino)pyrimidin-5-yl)(6-oxa-1-azaspiro[3.3]heptan-1-yl)-methanone;
(2-((3,5-diethoxybenzyl)amino)pyrimidin-5-yl)(6-oxa-1-azaspiro[3.3]heptan-1-yl)-methanone;
(2-((2,5-dichlorobenzyl)amino)pyrimidin-5-yl)(6-oxa-1-azaspiro[3.3]heptan-1-yl)-methanone;
(2-((2,3-dichlorobenzyl)amino)pyrimidin-5-yl)(6-oxa-1-azaspiro[3.3]heptan-1-yl)-methanone;
azetidin-1-yl(2-((5-chloro-2,3-dihydro-1H-inden-2-yl)amino)pyrimidin-5-yl)methanone;
(2-((5-chloro-2,3-dihydro-1H-inden-2-yl)amino)pyrimidin-5-yl)(2-(methoxymethyl)-azetidin-1-yl)methanone;
(2-((5-chloro-2,3-dihydro-1H-inden-2-yl)amino)pyrimidin-5-yl)((S)-2-(methoxymethyl)-azetidin-1-yl)methanone;
(2-((3,5-diisopropoxybenzyl)amino)pyrimidin-5-yl)(2-(2-hydroxypropan-2-yl)azetidin-1-yl)methanone;
(2-((3-cyclopropyl-5-isopropoxybenzyl)amino)pyrimidin-5-yl)(2-(2-hydroxypropan-2-yl)azetidin-1-yl)methanone;
(2-((5-chloro-2,3-dihydro-1H-inden-2-yl)amino)pyrimidin-5-yl)(2-(2-hydroxypropan-2-yl)azetidin-1-yl)methanone;
(2-((5-bromo-2,3-dihydro-1H-inden-2-yl)amino)pyrimidin-5-yl)(6-oxa-1-azaspiro[3.3]-heptan-1-yl)methanone;
(S)-(2-((5-bromo-2,3-dihydro-1H-inden-2-yl)amino)pyrimidin-5-yl)(6-oxa-1-azaspiro[3.3]heptan-1-yl)methanone;
(R)-(2-((5-bromo-2,3-dihydro-1H-inden-2-yl)amino)pyrimidin-5-yl)(6-oxa-1-azaspiro[3.3]heptan-1-yl)methanone;
(2-((3-(azetidin-1-yl)-5-isopropoxybenzyl)amino)pyrimidin-5-yl)(6-oxa-1-azaspiro[3.3]-heptan-1-yl)methanone;
(2-(2-hydroxypropan-2-yl)azetidin-1-yl)(2-((3-isopropyl-5-(trifluoromethoxy)benzyl)-amino)pyrimidin-5-yl)methanone;
(2-((3,5-dicyclopropylbenzyl)amino)pyrimidin-5-yl)(2-(2-hydroxypropan-2-yl)azetidin-1-yl)methanone;
(2-((3-ethoxy-5-(trifluoromethoxy)benzyl)amino)pyrimidin-5-yl)(2-(2-hydroxypropan-2-yl)azetidin-1-yl)methanone;
(2-((3-cyclopropyl-5-ethoxybenzyl)amino)pyrimidin-5-yl)(6-oxa-1-azaspiro[3.3]heptan-1-yl)methanone;
(2-((3-(azetidin-1-yl)-5-(trifluoromethoxy)benzyl)amino)pyrimidin-5-yl)(2-(2-hydroxy-propan-2-yl)azetidin-1-yl)methanone;
(2-((3-isopropyl-5-(trifluoromethoxy)benzyl)amino)pyrimidin-5-yl)(2-methylazetidin-1-yl)methanone;
(R)-(2-((3-isopropyl-5-(trifluoromethoxy)benzyl)amino)pyrimidin-5-yl)(2-methylazetidin-1-yl)methanone;
(S)-(2-((3-isopropyl-5-(trifluoromethoxy)benzyl)amino)pyrimidin-5-yl)(2-methylazetidin-1-yl)methanone;
6-oxa-1-azaspiro[3.3]heptan-1-yl(5-((3-(trifluoromethoxy)benzyl)amino)pyrazin-2-yl)-methanone;
6-oxa-1-azaspiro[3.3]heptan-1-yl(6-((3-(trifluoromethoxy)benzyl)amino)pyridazin-3-yl)-methanone;
6-oxa-1-azaspiro[3.3]heptan-1-yl(6-((3-(trifluoromethoxy)benzyl)amino)pyridin-3-yl)methanone;
azetidin-1-yl(5-((3-isopropyl-5-(trifluoromethoxy)benzyl)amino)pyrazin-2-yl)methanone;
(5-((3-isopropyl-5-(trifluoromethoxy)benzyl)amino)pyrazin-2-yl)(6-oxa-1-azaspiro[3.3]heptan-1-yl)methanone;
(6-((3-isopropyl-5-(trifluoromethoxy)benzyl)amino)pyridin-3-yl)(6-oxa-1-azaspiro[3.3]heptan-1-yl)methanone;
(6-((3-cyclopropyl-5-(trifluoromethoxy)benzyl)amino)pyridin-3-yl)(6-oxa-1-azaspiro[3.3]heptan-1-yl)methanone;
(6-((5-chloro-2,3-dihydro-1H-inden-2-yl)amino)pyridin-3-yl)(6-oxa-1-azaspiro[3.3]heptan-1-yl)methanone;
(2-((3-cyclopropyl-5-(trifluoromethoxy)benzyl)amino)pyrimidin-5-yl)(1-azaspiro[3.3]heptan-1-yl)methanone;
(2-((3-cyclopropyl-5-(trifluoromethoxy)benzyl)amino)pyrimidin-5-yl)(6-oxa-1-azaspiro[3.3]heptan-1-yl)methanone;
azetidin-1-yl(2-((3-cyclopropyl-5-(trifluoromethoxy)benzyl)amino)pyrimidin-5-yl)methanone;
(2-((3-cyclopropyl-5-(trifluoromethoxy)benzyl)amino)pyrimidin-5-yl)(2-oxa-6-azaspiro[3.3]heptan-6-yl)methanone;
(2-((3-cyclopropyl-5-(trifluoromethoxy)benzyl)amino)pyrimidin-5-yl)(3-hydroxyazetidin-1-yl)methanone;
(2-((3-cyclopropyl-5-(trifluoromethoxy)benzyl)amino)pyrimidin-5-yl)(3-(2-hydroxypropan-2-yl)azetidin-1-yl)methanone;
(2-((3-cyclopropyl-5-(trifluoromethoxy)benzyl)amino)pyrimidin-5-yl)(3-fluoroazetidin-1-yl)methanone;
(2-((3-cyclopropyl-5-(trifluoromethoxy)benzyl)amino)pyrimidin-5-yl)(3,3-difluoroazetidin-1-yl)methanone;
(2-((3-cyclopropyl-5-(trifluoromethoxy)benzyl)amino)pyrimidin-5-yl)(3-hydroxy-3-methylazetidin-1-yl)methanone;
(2-((3-cyclopropyl-5-(trifluoromethoxy)benzyl)amino)pyrimidin-5-yl)(3-(methylsulfonyl)azetidin-1-yl)methanone;
(2-((3-cyclopropyl-5-(trifluoromethoxy)benzyl)amino)pyrimidin-5-yl)(2,2-dimethylazetidin-1-yl)methanone;
(2-((3-cyclopropyl-5-(trifluoromethoxy)benzyl)amino)pyrimidin-5-yl)(2,3-dihydro-1H-pyrrolo[2,3-b]pyridin-1-yl)methanone;
(2-((3-cyclopropyl-5-(trifluoromethoxy)benzyl)amino)pyrimidin-5-yl)(2,3-dihydro-1H-pyrrolo[3,2-c]pyridin-1-yl)methanone;
tert-butyl 1-(2-((3-cyclopropyl-5-(trifluoromethoxy)benzyl)amino)pyrimidine-5-carbonyl)-1,6-diazaspiro[3.3]heptane-6-carboxylate;
(2-((3-cyclopropyl-5-(trifluoromethoxy)benzyl)amino)pyrimidin-5-yl)(2,4-dimethylazetidin-1-yl)methanone;
(2-((3-cyclopropyl-5-(trifluoromethoxy)benzyl)amino)pyrimidin-5-yl)(2-(methoxymethyl)pyrrolidin-1-yl)methanone;
(S)-(2-((3-cyclopropyl-5-(trifluoromethoxy)benzyl)amino)pyrimidin-5-yl)(2-(methoxymethyl)pyrrolidin-1-yl)methanone;
(R)-(2-((3-cyclopropyl-5-(trifluoromethoxy)benzyl)amino)pyrimidin-5-yl)(2-(methoxymethyl)pyrrolidin-1-yl)methanone;
(2-((3-cyclopropyl-5-(trifluoromethoxy)benzyl)amino)pyrimidin-5-yl)(2-oxa-5-azaspiro[3.4]octan-5-yl)methanone;
(2-((3-cyclopropyl-5-(trifluoromethoxy)benzyl)amino)pyrimidin-5-yl)(6-oxa-1-azaspiro[3.4]octan-1-yl)methanone;
(2-((3-cyclopropyl-5-(trifluoromethoxy)benzyl)amino)pyrimidin-5-yl)(2,3-dihydro-TH-pyrrolo[2,3-c]pyridin-1-yl)methanone;
(2-((3-cyclopropyl-5-(trifluoromethoxy)benzyl)amino)pyrimidin-5-yl)(3-(methoxymethyl)azetidin-1-yl)methanone;
(2-((3-cyclopropyl-5-(trifluoromethoxy)benzyl)amino)pyrimidin-5-yl)(3-((dimethylamino)methyl)azetidin-1-yl)methanone;
(2-((3-cyclopropyl-5-(trifluoromethoxy)benzyl)amino)pyrimidin-5-yl)(7-oxa-1-azaspiro[3.5]nonan-1-yl)methanone;
(2-((3-cyclopropyl-5-(trifluoromethoxy)benzyl)amino)pyrimidin-5-yl)(1,1-dioxido-1-thia-6-azaspiro[3.3]heptan-6-yl)methanone;
(2-((3-cyclopropyl-5-(trifluoromethoxy)benzyl)amino)pyrimidin-5-yl)(2-(methoxymethyl)azetidin-1-yl)methanone;
(R)-(2-((3-cyclopropyl-5-(trifluoromethoxy)benzyl)amino)pyrimidin-5-yl)(2-(methoxymethyl)azetidin-1-yl)methanone;
(S)-(2-((3-cyclopropyl-5-(trifluoromethoxy)benzyl)amino)pyrimidin-5-yl)(2-(methoxymethyl)azetidin-1-yl)methanone;
(2-((3-cyclopropyl-5-(trifluoromethoxy)benzyl)amino)pyrimidin-5-yl)(1-oxa-6-azaspiro[3.3]heptan-6-yl)methanone;
(2-((3-cyclopropyl-5-(trifluoromethoxy)benzyl)amino)pyrimidin-5-yl)(2,2-dioxido-2-thia-6-azaspiro[3.3]heptan-6-yl)methanone;
(2-((3-cyclopropyl-5-(trifluoromethoxy)benzyl)amino)pyrimidin-5-yl)(6,6-dioxido-6-thia-1-azaspiro[3.3]heptan-1-yl)methanone;
(2-((3-cyclopropyl-5-(trifluoromethoxy)benzyl)amino)pyrimidin-5-yl)(6-oxa-1-azaspiro[3.5]nonan-1-yl)methanone;
(2-((3-cyclopropyl-5-(trifluoromethoxy)benzyl)amino)pyrimidin-5-yl)(3-methoxyazetidin-1-yl)methanone;
(2-((3-cyclopropyl-5-(trifluoromethoxy)benzyl)amino)pyrimidin-5-yl)(3-(dimethylamino)azetidin-1-yl)methanone;
(2-((3-cyclopropyl-5-(trifluoromethoxy)benzyl)amino)pyrimidin-5-yl)(hexahydro-TH-furo[3,4-b]pyrrol-1-yl)methanone;
(2-((3-cyclopropyl-5-(trifluoromethoxy)benzyl)amino)pyrimidin-5-yl)(7-methyl-1,7-diazaspiro[3.5]nonan-1-yl)methanone;
(2-((3-cyclopropyl-5-(trifluoromethoxy)benzyl)amino)pyrimidin-5-yl)(2-(difluoromethyl)azetidin-1-yl)methanone;
(2-((3-cyclopropyl-5-(trifluoromethoxy)benzyl)amino)pyrimidin-5-yl)(2-(trifluoromethyl)azetidin-1-yl)methanone;
(2-((3-cyclopropyl-5-(trifluoromethoxy)benzyl)amino)pyrimidin-5-yl)(2-(2-hydroxypropan-2-yl)azetidin-1-yl)methanone;
(2-((3-cyclopropyl-5-(trifluoromethoxy)benzyl)amino)pyrimidin-5-yl)(6-oxa-1-azaspiro[3.4]octan-1-yl)methanone;
(R)-(2-((3-cyclopropyl-5-(trifluoromethoxy)benzyl)amino)pyrimidin-5-yl)(6-oxa-1-azaspiro[3.4]octan-1-yl)methanone;
(S)-(2-((3-cyclopropyl-5-(trifluoromethoxy)benzyl)amino)pyrimidin-5-yl)(6-oxa-1-azaspiro[3.4]octan-1-yl)methanone;
(2-((3-cyclopropyl-5-(trifluoromethoxy)benzyl)amino)pyrimidin-5-yl)(2,4-dimethylazetidin-1-yl)methanone;
(2-((3-cyclopropyl-5-(trifluoromethoxy)benzyl)amino)pyrimidin-5-yl)((2R,4R)-2,4-dimethylazetidin-1-yl)methanone;
(2-((3-cyclopropyl-5-(trifluoromethoxy)benzyl)amino)pyrimidin-5-yl)((2S,4S)-2,4-dimethylazetidin-1-yl)methanone;
(2-((3-cyclopropyl-5-(trifluoromethoxy)benzyl)amino)pyrimidin-5-yl)((2R,4S)-2,4-dimethylazetidin-1-yl)methanone;
(2-((3-cyclopropyl-5-(trifluoromethoxy)benzyl)amino)pyrimidin-5-yl)(6-oxa-1-azaspiro[3.5]nonan-1-yl)methanone;
(R)-(2-((3-cyclopropyl-5-(trifluoromethoxy)benzyl)amino)pyrimidin-5-yl)(6-oxa-1-azaspiro[3.5]nonan-1-yl)methanone;
(S)-(2-((3-cyclopropyl-5-(trifluoromethoxy)benzyl)amino)pyrimidin-5-yl)(6-oxa-1-azaspiro[3.5]nonan-1-yl)methanone;
(2-((3-ethyl-5-(trifluoromethoxy)benzyl)amino)pyrimidin-5-yl)(6-oxa-1-azaspiro[3.3]heptan-1-yl)methanone;
(2-((5-methyl-2,3-dihydro-1H-inden-2-yl)amino)pyrimidin-5-yl)(6-oxa-1-azaspiro[3.3]heptan-1-yl)methanone;
(2-((5-cyclopropyl-2,3-dihydro-1H-inden-2-yl)amino)pyrimidin-5-yl)(6-oxa-1-azaspiro[3.3]heptan-1-yl)methanone;
(2-((5-ethyl-2,3-dihydro-1H-inden-2-yl)amino)pyrimidin-5-yl)(6-oxa-1-azaspiro[3.3]heptan-1-yl)methanone;
(2-((5,6-dibromo-2,3-dihydro-1H-inden-2-yl)amino)pyrimidin-5-yl)(6-oxa-1-azaspiro[3.3]heptan-1-yl)methanone;
(2-((5,6-dimethyl-2,3-dihydro-1H-inden-2-yl)amino)pyrimidin-5-yl)(6-oxa-1-azaspiro[3.3]heptan-1-yl)methanone;
(2-((5-chloro-2,3-dihydro-1H-inden-2-yl)amino)pyrimidin-5-yl)(6-oxa-1-azaspiro[3.3]-heptan-1-yl)methanone;
(R)-(2-((5-chloro-2,3-dihydro-1H-inden-2-yl)amino)pyrimidin-5-yl)(6-oxa-1-azaspiro[3.3]heptan-1-yl)methanone;
(S)-(2-((5-chloro-2,3-dihydro-1H-inden-2-yl)amino)pyrimidin-5-yl)(6-oxa-1-azaspiro[3.3]heptan-1-yl)methanone;
(2-((5-chloro-2,3-dihydro-1H-inden-2-yl)amino)pyrimidin-5-yl)(2-(2-hydroxypropan-2-yl)azetidin-1-yl)methanone;
(2-(((R)-5-chloro-2,3-dihydro-1H-inden-2-yl)amino)pyrimidin-5-yl)(2-(2-hydroxypropan-2-yl)azetidin-1-yl)methanone;
(2-((5-chloro-2,3-dihydro-1H-inden-2-yl)amino)pyrimidin-5-yl)(6-oxa-1-azaspiro[3.4]octan-1-yl)methanone;
(2-(((R)-5-chloro-2,3-dihydro-1H-inden-2-yl)amino)pyrimidin-5-yl)(6-oxa-1-azaspiro[3.4]octan-1-yl)methanone;
(2-((5-chloro-2,3-dihydro-1H-inden-2-yl)amino)pyrimidin-5-yl)(2-methylazetidin-1-yl)-methanone;
(2-(((R)-5-chloro-2,3-dihydro-1H-inden-2-yl)amino)pyrimidin-5-yl)(2-methylazetidin-1-yl)methanone;
(2-((5-chloro-2,3-dihydro-1H-inden-2-yl)amino)pyrimidin-5-yl)(6-hydroxy-6-methyl-1-azaspiro[3.3]heptan-1-yl)methanone;
(R)-(2-((5-chloro-2,3-dihydro-1H-inden-2-yl)amino)pyrimidin-5-yl)(6-hydroxy-6-methyl-1-azaspiro[3.3]heptan-1-yl)methanone;
2-((5-chloro-2,3-dihydro-1H-inden-2-yl)amino)pyrimidin-5-yl)((S)-2-(2-hydroxypropan-2-yl)azetidin-1-yl)methanone;
2-(((R)-5-chloro-2,3-dihydro-1H-inden-2-yl)amino)pyrimidin-5-yl)((S)-2-(2-hydroxypropan-2-yl)azetidin-1-yl)methanone;
(2-((5-chloro-2,3-dihydro-1H-inden-2-yl)amino)pyrimidin-5-yl)((R)-2-(2-hydroxypropan-2-yl)azetidin-1-yl)methanone;
(2-(((R)-5-chloro-2,3-dihydro-1H-inden-2-yl)amino)pyrimidin-5-yl)((R)-2-(2-hydroxypropan-2-yl)azetidin-1-yl)methanone;
(2-((3-(methylsulfonyl)-5-(trifluoromethoxy)benzyl)amino)pyrimidin-5-yl)(6-oxa-1-azaspiro[3.3]heptan-1-yl)methanone;
(2-((5-(methylsulfonyl)-2,3-dihydro-1H-inden-2-yl)amino)pyrimidin-5-yl)(6-oxa-1-azaspiro[3.3]heptan-1-yl)methanone;
3-(((5-(6-oxa-1-azaspiro[3.3]heptane-1-carbonyl)pyrimidin-2-yl)amino)methyl)-5-(trifluoromethoxy)benzonitrile;
3-(((5-(6-oxa-1-azaspiro[3.3]heptane-1-carbonyl)pyrimidin-2-yl)amino)methyl)-5-(trifluoromethoxy)benzamide;
(2-((3-cyclopropyl-5-(trifluoromethoxy)benzyl)amino)pyrimidin-5-yl)(1,6-diazaspiro[3.3]heptan-1-yl)methanone;
(2-((3-cyclopropyl-5-(trifluoromethoxy)benzyl)amino)pyrimidin-5-yl)(6-methyl-1,6-diazaspiro[3.3]heptan-1-yl)methanone;
(2-((3-cyclopropyl-5-(trifluoromethoxy)benzyl)amino)pyrimidin-5-yl)(6-ethyl-1,6-diazaspiro[3.3]heptan-1-yl)methanone;
(6-cyclobutyl-1,6-diazaspiro[3.3]heptan-1-yl)(2-((3-cyclopropyl-5-(trifluoromethoxy)-benzyl)amino)pyrimidin-5-yl)methanone;
(2-((3-cyclopropyl-5-(trifluoromethoxy)benzyl)amino)pyrimidin-5-yl)(6-(oxetan-3-yl)-1,6-diazaspiro[3.3]heptan-1-yl)methanone;
(2-((3-cyclopropyl-5-(trifluoromethoxy)benzyl)amino)pyrimidin-5-yl)(6-cyclopropyl-1,6-diazaspiro[3.3]heptan-1-yl)methanone;
(2-((3-cyclopropyl-5-(trifluoromethoxy)benzyl)amino)pyrimidin-5-yl)(6-isopropyl-1,6-diazaspiro[3.3]heptan-1-yl)methanone;
(2-((3-cyclopropyl-5-(trifluoromethoxy)benzyl)amino)pyrimidin-5-yl)(6-(2,2,2-trifluoroethyl)-1,6-diazaspiro[3.3]heptan-1-yl)methanone;
(2-((3-cyclopropyl-5-(trifluoromethoxy)benzyl)amino)pyrimidin-5-yl)(6-(2,2-difluoroethyl)-1,6-diazaspiro[3.3]heptan-1-yl)methanone;
(2-((3-cyclopropyl-5-(trifluoromethoxy)benzyl)amino)pyrimidine-5-yl)(6-(2-(methylsulfonyl)ethyl)-1,6-diazaspiro[3.3]heptan-1-yl)methanone;
(2-((3-isopropyl-5-(trifluoromethoxy)benzyl)amino)pyrimidin-5-yl)(6-(methylsulfonyl)-1,6-diazaspiro[3.3]heptan-1-yl)methanone;
(2-((5-chloro-2,3-dihydro-1H-inden-2-yl)amino)pyrimidin-5-yl)(6-(methylsulfonyl)-1,6-diazaspiro[3.3]heptan-1-yl)methanone;
1-(1-(2-((3-cyclopropyl-5-(trifluoromethoxy)benzyl)amino)pyrimidine-5-carbonyl)-1,6-diazaspiro[3.3]heptan-6-yl)ethanone;
1-(1-(2-((5-chloro-2,3-dihydro-1H-inden-2-yl)amino)pyrimidine-5-carbonyl)-1,6-diazaspiro[3.3]heptan-6-yl)ethanone;
(2-((3-cyclopropyl-5-(trifluoromethoxy)benzyl)amino)pyrimidin-5-yl)(6-methyl-1,6-diazaspiro[3.4]octan-1-yl)methanone;
(2-((3-cyclopropyl-5-(trifluoromethoxy)benzyl)amino)pyrimidin-5-yl)(6-methyl-1,6-diazaspiro[3.5]nonan-1-yl)methanone;
(2-((3-cyclopropyl-5-(trifluoromethoxy)benzyl)amino)pyrimidin-5-yl)(2-methyl-2,5-diazaspiro[3.4]octan-5-yl)methanone;
(2-((3-(azetidin-1-yl)-5-(trifluoromethoxy)benzyl)amino)pyrimidin-5-yl)(6-oxa-1-azaspiro[3.3]heptan-1-yl)methanone;
(2-((3-ethoxy-5-isopropoxybenzyl)amino)pyrimidin-5-yl)(6-oxa-1-azaspiro[3.3]heptan-1-yl)methanone;
(2-((3-ethoxy-5-isopropoxybenzyl)amino)pyrimidin-5-yl)(2-(2-hydroxypropan-2-yl)azetidin-1-yl)methanone;
(2-((5-isopropoxy-2,3-dihydro-1H-inden-2-yl)amino)pyrimidin-5-yl)(6-oxa-1-azaspiro[3.3]heptan-1-yl)methanone; and
(2-((5-ethoxy-2,3-dihydro-1H-inden-2-yl)amino)pyrimidin-5-yl)(6-oxa-1-azaspiro[3.3]heptan-1-yl)methanone,

or a pharmaceutically acceptable salt, solvate, or solvate of the salt of any of the foregoing.

Some embodiments provide a pharmaceutical composition comprising a compound of formula (I), or a pharmaceutically acceptable salt thereof, and a pharmaceutically acceptable carrier.

Some embodiments provide a method for the treatment or prophylaxis of renal conditions, liver conditions, inflammatory conditions, conditions of the nervous system, conditions of the respiratory system, vascular and cardiovascular conditions, fibrotic diseases, cancer, ocular conditions, metabolic conditions, cholestatic and other forms of chronic pruritus and acute and chronic organ transplant rejection, which comprises administering to a patient in need of such treatment a therapeutically effective amount of a compound of formula (I) or a pharmaceutically acceptable salt, solvate or salt of a solvate thereof.

Some embodiments provide a method for the treatment or prophylaxis of multiple sclerosis which comprises administering to a patient in need of such treatment a therapeutically effective amount of a compound of formula (I) or a pharmaceutically acceptable salt, solvate or salt of a solvate thereof.

Any of the features of an embodiment is applicable to all embodiments identified herein. Moreover, any of the features of an embodiment is independently combinable, partly or wholly with other embodiments described herein in any way, e.g., one, two, or three or more embodiments may be combinable in whole or in part. Further, any of the features of an embodiment may be made optional to other embodiments. Any embodiment of a method can comprise another embodiment of a compound, and any embodiment of a compound can be configured to perform a method of another embodiment.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The term “patient” includes mammals such as mice, rats, cows, sheep, pigs, rabbits, goats, horses, monkeys, dogs, cats, and humans. In some embodiments, the patient is a human.

The term “halo” or “halogen” refers to any radical of fluorine, chlorine, bromine or iodine.

The term “alkyl” refers to a saturated hydrocarbon chain that may be a straight chain or branched chain, containing the indicated number of carbon atoms. For example, C₁₋₆ alkyl indicates that the group may have from 1 to 6 (inclusive) carbon atoms in it. In some embodiments, an alkyl is a C₁₋₆ alkyl which represents a straight-chain or branched saturated hydrocarbon radical having 1 to 6 carbon atoms. Examples of alkyl include without limitation methyl, ethyl, n-propyl, isopropyl, n-butyl, isobutyl, sec-butyl, and tert-butyl.

The term “cycloalkyl” refers to a fully saturated monocyclic, bicyclic, tricyclic or other polycyclic hydrocarbon group having the indicated number of ring carbon atoms. Multicyclic cycloalkyl may be fused, bridged or spiro ring systems. Cycloalkyl groups include, but are not limited to, cyclopropyl, cyclobutyl, cyclopentyl, cyclohexyl, cycloheptyl, cyclooctyl and norbornyl. In some embodiments, cycloalkyl is a monocyclic C₃-C₈ cycloalkyl.

The term “haloalkyl” refers to an alkyl group in which at least one hydrogen atom is replaced by halo. In some embodiments, more than one hydrogen atom (e.g., 2, 3, 4, 5 or 6) are replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Examples of haloalkyl also include “fluoroalkyl”, that is an alkyl group in which one or more hydrogen atoms are replaced with fluorine.

As referred to herein, the term “alkoxy” refers to a group of formula —O-(alkyl). Alkoxy can be, for example, methoxy, ethoxy, propoxy, isopropoxy, butoxy, iso-butoxy, sec-butoxy, pentoxy, 2-pentoxy, 3-pentoxy, or hexyloxy. Likewise, the term “thioalkoxy” refers to a group of formula —S-(alkyl). The terms “haloalkoxy” and “thiohaloalkoxy” refer to —O-(haloalkyl) and —S-(haloalkyl), respectively.

In any of the aforementioned groups, one or more hydrogen atoms in the alkyl portion of the group may be replaced with deuterium, for example, a deutero methoxy group (—OCD₃) or a deutero methyl group (—CD₃).

The term “aralkyl” refers to an alkyl moiety in which an alkyl hydrogen atom is replaced by an aryl group. One of the carbons of the alkyl moiety serves as the point of attachment of the aralkyl group to another moiety. Non-limiting examples of “aralkyl” include benzyl, 2-phenylethyl, and 3-phenylpropyl groups.

The term “alkenyl” refers to a straight or branched hydrocarbon chain containing the indicated number of carbon atoms and having one or more carbon-carbon double bonds. Alkenyl groups can include, e.g., vinyl, allyl, 1-butenyl, and 2-hexenyl. In some embodiments, an alkenyl is a C₂-C₆ alkenyl.

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. Cycloalkenyl moieties can include, e.g., cyclopentenyl, cyclohexenyl, cyclohexadienyl, or norbornenyl.

The term “alkynyl” refers to a straight or branched hydrocarbon chain containing the indicated number of carbon atoms and having one or more carbon-carbon triple bonds. Alkynyl groups can include, e.g., ethynyl, propargyl, 1-butynyl, and 2-hexynyl. In some embodiments, an alkynyl is a C₂-C₆ alkynyl.

The term “heterocycle”, “heterocyclyl” or “heterocyclic” as used herein except where noted, represents a stable 4-, 5-, 6- or 7-membered monocyclic- or a stable 6-, 7-, 8-, 9-, 10-, 11-, or 12-membered bicyclic heterocyclic ring system which comprises at least one non-aromatic (i.e. saturated or partially unsaturated) ring which consists of carbon atoms and from one to four, preferably up to three, heteroatoms selected from the group consisting of N, O and S, wherein the nitrogen and sulfur atoms may optionally be oxidized as N-oxide, sulfoxide or sulfone, and wherein the nitrogen atom may optionally be quaternized. A heterocycle can be bonded via a ring carbon atom or, if available, via a ring nitrogen atom. Bicyclic heterocyclic ring systems may be fused, bridged, or spiro bicyclic heterocyclic ring system(s). In some embodiments, heterocyclyl is monocyclic having 4 to 7, preferably 4 to 6, ring atoms, of which 1 or 2 are heteroatoms independently selected from the group consisting of N, O and S. In some embodiments, a heterocyclyl group is bicyclic, and in which case, the second ring may be an aromatic or a non-aromatic ring which consists of carbon atoms and from one to four, preferably up to three, heteroatoms independently selected from the group consisting of N, O and S, or the second ring may be a benzene ring, or a “cycloalkyl”, or a “cycloalkenyl”, as defined herein. Examples of such heterocyclic groups include, but are not limited to azetidine, chroman, dihydrofuran, dihydropyran, dioxane, dioxolane, hexahydroazepine, imidazolidine, imidazoline, indoline, isochroman, isoindoline, isothiazoline, isothiazolidine, isoxazoline, isoxazolidine, morpholine, oxazoline, oxazolidine, oxetane, piperazine, piperidine, dihydropyridine, tetrahydropyridine, dihydropyridazine, pyran, pyrazolidine, pyrazoline, pyrrolidine, pyrroline, tetrahydrofuran, tetrahydropyran, thiamorpholine, tetrahydrothiophene, thiazoline, thiazolidine, thiomorpholine, thietane, thiolane, sulfolane, 1,3-dioxolane, 1,3-oxazolidine, 1,3-thiazolidine, tetrahydrothiopyran, tetrahydrotriazine, 1,3-dioxane, 1,4-dioxane, hexahydrotriazine, tetrahydro-oxazine, tetrahydropyrimidine, perhydroazepine, perhydro-1,4-diazepine, perhydro-1,4-oxazepine, 7-azabicyclo[2.2.1]heptane, 3-azabicyclo[3.2.0]heptane, 7-azabicyclo[4.1.0]heptane, 2,5-diazabicyclo[2.2.1]heptane, 2-oxa-5-azabicyclo[2.2.1]heptane, tropane, 2-oxa-6-azaspiro[3.3]heptane, dihydrobenzofuran, diydrobenzimidazolyl, dihydrobenzoxazole, and dihydrobenzothiazolyl, and N-oxides or sulfones or sulfoxides thereof.

The term “aryl” as used herein, is intended to mean any stable monocyclic or bicyclic carbon ring of up to 6 members in each ring (i.e., 6 to 10 total ring atoms) wherein at least one ring is aromatic. For example, a C₆-C₁₀ aryl group such as phenyl, naphthyl, tetrahydronaphthyl, indanyl, or 1H-indenyl.

The term “heteroaryl”, as used herein except where noted, represents a stable 5-6- or 7-membered monocyclic- or stable 9- or 10-membered fused bicyclic ring system which comprises at least one aromatic ring, which consists of carbon atoms and from one to four, preferably up to three, heteroatoms selected from the group consisting of N, O and S wherein the nitrogen and sulfur heteroatoms may optionally be oxidized, and the nitrogen heteroatom may optionally be quaternized. In the case of a “heteroaryl” which is a bicyclic group, the second ring need not be aromatic and need not comprise a heteroatom. Accordingly, bicyclic “heteroaryl” includes, for example, a stable 5- or 6-membered monocyclic aromatic ring consisting of carbon atoms and from one to four, preferably up to three, heteroatoms, as defined immediately above, fused to a benzene ring, or a second monocyclic “heteroaryl”, or a “heterocyclyl”, a “cycloalkyl”, or a “cycloalkenyl”, as defined above. Examples of heteroaryl groups include, but are not limited to, benzimidazole, benzopyrazole, benzisothiazole, benzisoxazole, benzofuran, isobenzofuran, benzothiazole, benzothiophene, benzotriazole, benzoxazole, cinnoline, furan, furazan, imidazole, indazole, indole, indolizine, isoquinoline, isothiazole, isoxazole, naphthyridine, oxadiazole, oxazole, phthalazine, pteridine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, benzimidazole, benzothiadiazole, isoindole, pyrrolopyridines, imidazopyridines such as imidazo[1,2-a]pyridine, pyrazolopyridine, pyrrolopyrimidine and N-oxides thereof.

The term “treating”, “treat”, or “treatment” refers generally to controlling, alleviating, ameliorating, slowing the progress of or eliminating a named condition once the condition has been established. In addition to its customary meaning, the term “prophylaxis”, “prophylactic”, “preventing”, “prevent”, or “prevention” also refers to delaying the onset of, or reducing the risk of developing a named condition or of a process that can lead to the condition, or the recurrence of symptoms of a condition.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

### Compound Forms and Salts

The compounds of this disclosure may contain one or more asymmetric centers and thus occur as racemates and racemic mixtures, enantiomerically enriched mixtures, single enantiomers, individual diastereomers and diastereomeric mixtures. The compounds of the present disclosure may, either by nature of asymmetric centers or by restricted rotation, be present in the form of isomers (e.g., enantiomers, diastereomers).

It will also be appreciated that when two or more asymmetric centers are present in the compounds of the disclosure, several diastereomers and enantiomers of the exemplified structures will often be possible, and that pure diastereomers and pure enantiomers represent preferred embodiments. It is intended that pure stereoisomers, pure diastereomers, pure enantiomers, and mixtures thereof, are within the scope of the disclosure.

All isomers, whether separated, pure, partially pure, or in racemic mixture, of the compounds of this disclosure are encompassed within the scope of this disclosure. The purification of said isomers and the separation of said isomeric mixtures may be accomplished by standard techniques known in the art. For example, diastereomeric mixtures can be separated into the individual isomers by chromatographic processes or crystallization, and racemates can be separated into the respective enantiomers either by chromatographic processes on chiral phases or by resolution.

The compounds of the present disclosure include all cis, trans, syn, anti, entgegen (E), and zusammen (Z) isomers as well as mixtures thereof. The compounds of the present disclosure may also be represented in multiple tautomeric forms, in such instances, the present disclosure expressly includes all tautomeric forms of the compounds described herein, even though only a single tautomeric form may be represented. In addition, where a term used in the present disclosure encompasses a group that may tautomerize, all tautomeric forms are expressly included thereunder. For example, hydroxy substituted heteroaryl groups include, but are not limited to, 2-hydroxypyridine as well as 2-pyridone, 1-hydroxyisoquinoline as well as 1-oxo-1,2-dihyroisoquinoline, 2-hydroxypyrimidine as well as 2-pyrimidone, 2-hydroxyquinoline as well as 2-quinolinone, 5-hydroxy-1,2,4-oxadiazole as well as 1,2,4-oxadiazole-5(4H)one, and the like. All such isomeric forms of such compounds are expressly included in the present disclosure.

The compounds of the present disclosure include the compounds themselves, as well as their salts, solvate, and solvate of the salt, if applicable. Salts for the purposes of the present disclosure are preferably pharmaceutically acceptable salts of the compounds according to the present disclosure. Salts which are not themselves suitable for pharmaceutical uses but can be used, for example, for isolation or purification of the compounds according to the disclosure are also included. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. When the compound of the present disclosure is basic, pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfonic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, naphthalenedisulfonic, methanesulfonic, ethanesulfonic, ethanedisulfonic, camphorsulfonic, gluconic, mandelic, mucic, pantothenic, oxalic, isethionic, and the like.

When the compound of the present disclosure is acidic, salts may be prepared from pharmaceutically acceptable non-toxic bases, including inorganic and organic bases. Such salts that may be prepared include lithium salt, sodium salt, potassium salt, magnesium salt, calcium salt, dicyclohexylamine salt, N-methyl-D-glucamine salt, tris(hydroxymethyl)methylamine salt, arginine salt, lysine salt, and the like.

Lists of suitable salts may be found in Remington's Pharmaceutical Sciences, 17th ed., Mack Publishing Company, Easton, Pa., 1985, p. 1418; S. M. Berge et al., “Pharmaceutical Salts”, J. Pharm. Sci. 1977, 66, 1-19; and “Pharmaceutical Salts: Properties, Selection, and Use. A Handbook”; Wermuth, C. G. and Stahl, P. H. (eds.) Verlag Helvetica Chimica Acta, Zurich, 2002 [ISBN 3-906390-26-8]; each of which is incorporated herein by reference in its entirety.

Solvates in the context of the present disclosure are designated as those forms of the compounds according to the present disclosure which form a complex in the solid or liquid state by stoichiometric coordination with solvent molecules. Hydrates are a specific form of solvates, in which the coordination takes place with water. Hydrates are preferred solvates in the context of the present disclosure. The formation of solvates is described in greater detail in “Solvents and Solvent Effects in Organic Chemistry”; Reichardt, C. and Welton T.; John Wiley & Sons, 2011 [ISBN: 978-3-527-32473-6], the contents of which is incorporated herein by reference in its entirety. A person of ordinary skill in the art would recognize the solvates of the present disclosure.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶CL, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Due to comparatively easy preparability and detectability, especially compounds labeled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. Such modifications of the compounds according to the present disclosure may therefore in some cases also constitute a preferred embodiment of the present disclosure. In some embodiments, hydrogen atoms of the compounds described herein may be replaced with deuterium atoms. Isotopic variants of the compounds according to the present disclosure can be prepared by processes known to those skilled in the art, for example by the methods described below and the methods described in the working examples, by using corresponding isotopic modifications of the particular reagents and/or starting compounds therein.

### Pharmaceutical Compositions

The term “pharmaceutical composition” as used herein is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt, or solvate or solvate of the salt thereof, and a pharmaceutically acceptable carrier.

The term “pharmaceutically acceptable carrier” refers to a carrier or an adjuvant that may be administered to a patient, together with a compound of the present disclosure, or a pharmaceutically acceptable salt, solvate, or salt of the solvate thereof, and which does not destroy the pharmacological activity thereof and is nontoxic when administered in doses sufficient to deliver a therapeutic amount of the compound.

In some embodiments, the compounds of the present application are administered at about 1 mg to 1,000 mg, about 2 mg to 900 mg, about 3 mg to 800 mg, about 4 mg to 700 mg, about 5 mg to 600 mg, about 10 mg to 500 mg, about 50 mg to 400 mg, about 100 mg to 300 mg, about 150 mg to 250 mg, or any value in between. In some embodiments, the total daily dosage may be divided and administered in portions during the day, for example, once per day, twice per day, three times per day or four times per day. In some embodiments, the total dosage may be administered once per week, twice per week, three times per week, four times per week, five times per week or six times per week.

In some embodiments, the pharmaceutical compositions of the present disclosure for injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

In some embodiments, the pharmaceutical compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin. If desired, and for more effective distribution, the compounds can be incorporated into slow release or targeted delivery systems such as polymer matrices, liposomes, and microspheres.

In some embodiments, the pharmaceutical compositions that are injectable formulations can be sterilized, for example, by filtration through a bacterial-retaining filter, or by incorporating sterilizing agents in the form of sterile solid pharmaceutical compositions that can be dissolved or dispersed in sterile water or other sterile injectable mediumjust prior to use.

In some embodiments, solid dosage forms of the instant pharmaceutical compositions for oral administration. In some embodiments, the oral dosage forms include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid pharmaceutical compositions of a similar type may also be employed as fillers in soft and hard-filled gelatin capsules using such excipients as lactose or milk sugar as well as high molecular weight polyethylene glycols and the like.

The solid dosage forms of the instant pharmaceutical compositions of tablets, dragées, capsules, pills, and granules can be prepared with coatings and shells such as enteric coatings and other coatings well known in the pharmaceutical formulating art. They may optionally contain opacifying agents and can also be of a formulation that they release the active ingredient(s) only, or preferentially, in a certain part of the intestinal tract, optionally, in a delayed manner. Examples of embedding pharmaceutical compositions which can be used include polymeric substances and waxes.

The active compounds can also be in microencapsulated form, if appropriate, with one or more of the above-mentioned excipients.

Some embodiments provide liquid dosage forms of the instant pharmaceutical compositions for oral administration. In some embodiments, the liquid dosages include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral pharmaceutical compositions can also include adjuvants such as wetting agents, emulsifying and suspending agents, sweetening, flavoring, and perfuming agents.

Suspensions of the instant compounds, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Pharmaceutical compositions for rectal or vaginal administration are preferably suppositories which can be prepared by mixing the compounds with suitable non-irritating excipients or carriers such as cocoa butter, polyethylene glycol or a suppository wax which are solid at RT but liquid at body temperature and therefore melt in the rectum or vaginal cavity and release the active compound.

Dosage forms for topical administration of a compound or pharmaceutical composition of the present disclosure include powders, patches, sprays, ointments and inhalants. The active compound is mixed under sterile conditions with a pharmaceutically acceptable carrier and any needed preservatives, buffers, or propellants which may be required.

### Uses

Some embodiments provide a method for the treatment or prophylaxis of a disease, disorder, or condition selected from the group consisting of: renal conditions, liver conditions, inflammatory conditions, conditions of the nervous system, conditions of the respiratory system, vascular and cardiovascular conditions, fibrotic diseases, cancer, ocular conditions, metabolic conditions, cholestatic and other forms of chronic pruritus and acute and chronic organ transplant rejection, comprising administering to a patient in need of such treatment a therapeutically effective amount of a compound of formula (I), or a pharmaceutically acceptable salt, solvate or salt of a solvate thereof, or a pharmaceutical composition comprising a compound of formula (I), or a pharmaceutically acceptable salt, solvate or salt of a solvate thereof.

Renal conditions include, but are not limited to, acute kidney injury and chronic renal disease with and without proteinuria including end-stage renal disease (ESRD). In more detail, this includes decreased creatinine clearance and decreased glomerular filtration rate, microalbuminuria, albuminuria and proteinuria, glomerulosclerosis with expansion of reticulated mesangial matrix with or without significant hypercellularity (particularly diabetic nephropathy and amyloidosis), focal thrombosis of glomerular capillaries (particularly thrombotic microangiopathies), global fibrinoid necrosis, ischemic lesions, malignant nephrosclerosis (such as ischemic retraction, reduced renal blood flow and renal arteriopathy), swelling and proliferation of intracapillary (endothelial and mesangial) and/or extracapillary cells (crescents) like in glomerular nephritis entities, focal segmental glomerular sclerosis, IgA nephropathy, vasculitides/systemic diseases as well as acute and chronic kidney transplant rejection.

Liver conditions include, but are not limited to, liver cirrhosis, hepatic congestion, cholestatic liver disease including pruritus, nonalcoholic steatohepatitis and acute and chronic liver transplant rejection.

Inflammatory conditions include, but are not limited to, arthritis, osteoarthritis, multiple sclerosis, systemic lupus erythematodes, inflammatory bowel disease, abnormal evacuation disorder and the like as well as inflammatory airways diseases such as idiopathic pulmonary fibrosis (IPF), chronic obstructive pulmonary disease (COPD) or chronic asthma bronchiale.

Further conditions of the respiratory system include, but are not limited to, other diffuse parenchymal lung diseases of different etiologies including iatrogenic drug-induced fibrosis, occupational and/or environmental induced fibrosis, systemic diseases and vasculitides, granulomatous diseases (sarcoidosis, hypersensitivity pneumonia), collagen vascular disease, alveolar proteinosis, Langerhans cell granulomatosis, lymphangioleiomyomatosis, inherited diseases (Hermansky-Pudlak Syndrome, tuberous sclerosis, neurofibromatosis, metabolic storage disorders, familial interstitial lung disease), radiation induced fibrosis, silicosis, asbestos induced pulmonary fibrosis or acute respiratory distress syndrome (ARDS).

Conditions of the nervous system include, but are not limited to, neuropathic pain, schizophrenia, neuro-inflammation (e.g. astrogliosis), peripheral and/or autonomic (diabetic) neuropathies, multiple sclerosis, and the like.

Vascular conditions include, but are not limited to, atherosclerosis, thrombotic vascular disease as well as thrombotic microangiopathies, proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction and the like.

Cardiovascular conditions include, but are not limited to, acute coronary syndrome, coronary heart disease, myocardial infarction, arterial and pulmonary hypertension, cardiac arrhythmia such as atrial fibrillation, stroke and other vascular damage.

Fibrotic diseases include, but are not limited to myocardial and vascular fibrosis, renal fibrosis, liver fibrosis, pulmonary fibrosis, skin fibrosis, scleroderma and encapsulating peritonitis.

Cancer and cancer metastasis include, but are not limited to, breast cancer, ovarian cancer, lung cancer, prostate cancer, mesothelioma, glioma, hepatic carcinoma, gastrointestinal cancers and progression and metastatic aggressiveness thereof.

Ocular conditions include, but are not limited to, proliferative and non-proliferative (diabetic) retinopathy, dry and wet age-related macular degeneration (AMD), macular edema, central arterial/venous occlusion, traumatic injury, glaucoma and the like. Particularly, the ocular condition is glaucoma.

Metabolic conditions include, but are not limited to, obesity and diabetes.

Accordingly, the present disclosure further provides a method for the treatment or prophylaxis of renal conditions, liver conditions, inflammatory conditions, conditions of the nervous system, conditions of the respiratory system, vascular and cardiovascular conditions, fibrotic diseases, cancer, ocular conditions, metabolic conditions, cholestatic and other forms of chronic pruritus and acute and chronic organ transplant rejection, which comprises administering to a patient in need of such treatment a therapeutically effective amount of a compound of formula (I) or a pharmaceutically acceptable salt, solvate or salt of a solvate thereof.

In some embodiments the condition to be treated is multiple sclerosis, including relapsing-remitting multiple sclerosis, relapsing multiple sclerosis, primary-progressive multiple sclerosis, or secondary-progressive multiple sclerosis. In some embodiments the administration of a compound of formula (I) attenuates, reverses, or inhibits demyelination in the patient being treated. In some embodiments the administration of a compound of formula (I) promotes remyelination in the patient being treated. In some embodiments, demyelination or remyelination may be monitored by magnetic resonance including imaging (MRI) including conventional T₁-weighted and T₂-weighted imaging, magnetic resonance spectroscopy, diffusion tensor imaging (DTI), magnetization transfer imaging and separation of T₂ relaxation components. In some embodiments, demyelnation or remyelination may be monitored by ultrashort echo time (TE) imaging or ³¹P spectroscopy.

### Administration

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, or any value in between (e.g., from about 0.01 to about 100 mg/kg, from about 0.1 to about 100 mg/kg, from about 1 to about 100 mg/kg, from about 1 to about 10 mg/kg, or any value in between) every 4 to 120 hours, or any value in between. The interrelationship of dosages for animals and humans (based on milligrams per meter squared of body surface) is described by Freireich et al., Cancer Chemother. Rep. 50, 219-244 (1966) and is understood by those skilled in the art. Body surface area may be approximately determined from height and weight of the patient by those skilled in the art. See, e.g., Scientific Tables, Geigy Pharmaceuticals, Ardsley, N.Y., 537 (1970). In certain embodiments, the compositions are administered by oral administration or by injection. The methods herein contemplate administration of an effective amount of compound or compound composition to achieve the desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, and the judgment of the treating physician.

In some embodiments, dosage forms include from about 0.001 milligrams to about 2,000 milligrams, or any value in between (including, from about 0.001 milligrams to about 1,000 milligrams, from about 0.001 milligrams to about 500 milligrams, from about 0.01 milligrams to about 250 milligrams, from about 0.01 milligrams to about 100 milligrams, from about 0.05 milligrams to about 50 milligrams, and from about 0.1 milligrams to about 25 milligrams, or any value in between) of a compound of Formula (I) (and/or a compound of any of the other formulae described herein) or a salt (e.g., a pharmaceutically acceptable salt) thereof as defined anywhere herein. The dosage forms can further include a pharmaceutically acceptable carrier and/or an additional therapeutic agent.

Appropriate dosage levels may be determined by any suitable method known to one skilled in the medical arts. Preferably, the active substance is administered at a frequency of 1 to 4 times per day for topical administration, or less often if a drug delivery system is used.

Nevertheless, actual dosage levels and time course of administration of the active ingredients in the pharmaceutical compositions of the present disclosure may be varied so as to obtain an amount of the active ingredient which is effective to achieve the desired therapeutic response for a particular patient, composition and mode of administration, without being toxic to the patient. It may therefore be necessary where appropriate to deviate from the stated amounts, in particular as a function of age, gender, body weight, diet and general health status of the patient, route of administration, individual response to the active ingredient, nature of the preparation, and time or interval over which administration takes place. Thus, it may be satisfactory in some cases to manage with less than the aforementioned minimum amount, whereas in other cases the stated upper limit must be exceeded. It may in the event of administration of larger amounts be advisable to divide these into multiple individual doses spread over the day.

### Combinations

In some embodiments, compounds of formula (I), or a pharmaceutically acceptable salt, solvate, or salt of the solvate thereof, may be co-administered with one or more additional therapeutic agents. The additional therapeutic agents include, but are not limited to corticosteroids, interferons, monoclonal antibodies, and immunomodulators. For example, methylprednisolone; interferon beta-1a, interferon beta-lb; natalizumab, alemtuzumab, daclizumab, ocrelizumab; glatiramer acetate, mitoxantrone, fingolimod, teriflunomide, cladribine and dimethyl fumarate.

In some embodiments, compounds of formula (I), or a pharmaceutically acceptable salt, solvate, or salt of the solvate thereof, may be administered to a subject undergoing plasmapheresis.

### Synthesis

The preparation of compounds of formula (I) of the present disclosure may be carried out in sequential or convergent synthetic routes. Syntheses of the disclosure are shown in the following general schemes. The skills required for carrying out the reactions and purifications of the resulting products are known to those persons skilled in the art. In case a mixture of enantiomers or diastereoisomers is produced during a reaction, these enantiomers or diastereoisomers can be separated by methods described herein or known to the man skilled in the art such as e.g. (chiral) chromatography or crystallization. The substituents and indices used in the following description of the processes have the significance given herein.

Compounds of formula (I) may be prepared as illustrated in Scheme 1. A halogenated heteroarylcarboxylic acid or carboxylate 1 is reacted with an amine 2 in the presence of a base, followed by hydrolysis in the case of carboxylate, to provide the amino heteroarylcarboxylic acid 3. The acid 3 is coupled with a cyclic amine 4 using standard amide coupling techniques (for example, HATU, EDCI, DCC, HOBt/HBTU with an organic base like triethylamine or diisopropylethyl amine) to provide the corresponding compound of formula (I).

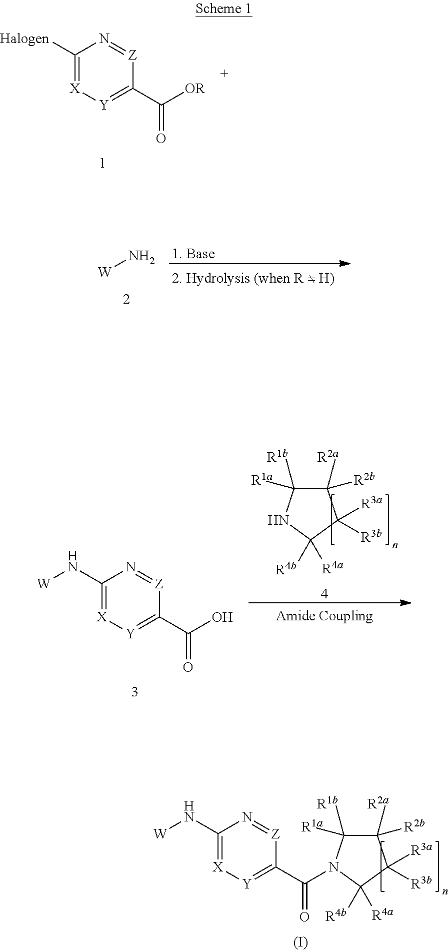

Amines 2 are commercially available or are accessible from commercially available precursors using conventional synthetic methodologies. Scheme 2 depicts some representative methods for the preparation of amines 2 wherein W is

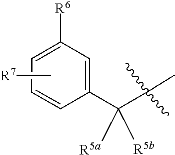

from aryl halides and aryl carboxylates. Aryl halide 5 can be converted to the corresponding aldehyde 6 by treatment with an organolithium reagent (e.g., nBuLi) and dimethylformamide; aldehyde 6 can also be prepared by reducing the aryl carboxylate 7 to the corresponding alcohol using e.g., LiBH₄, and oxidizing the latter to the aldehyde using e.g., Dess-Martin periodinane. Reductive amination (e.g., hydroxylamine followed by Pd/C and H₂) of 6 leads to the amine 2a. Aryl carboxylate 7 can be converted to the corresponding amide 8 using ammonia, and reduction of 8 using e.g., lithium aluminum hydride provides the amine 2a.

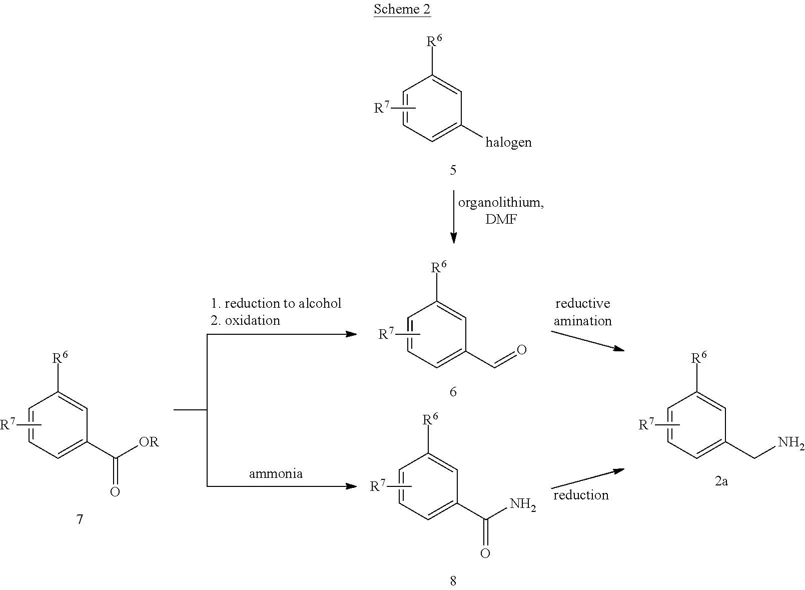

Intermediate halides 5, aldehydes 6 and carboxylates 7 are either commercially available or may be prepared from commercially available precursors as illustrated in Scheme 3. Metal-catalyzed cross coupling reactions may be used to introduce a variety of substituents on to the aromatic ring. For example, formation of C—C bonds may be achieved by reacting an aryl halide 9 with a Grignard reagent in the presence of nickel catalyst (Kumada reaction), or with an organozinc halide in the presence of palladium catalyst (Negishi reaction), or with a boronate or organic boronic acid in the presence of palladium catalyst (Suzuki-Miyaura reaction); aryl halides 9 may also be converted to arylamines under Buchwald-Hartwig reaction conditions (e.g., in the presence of palladium catalyst). Nucleophilic substitution reactions may be used to prepare additional intermediates. For example, phenoxide generated from phenol 10 upon treatment with a base is reacted with alkyl-LG (LG=leaving group) to provide the corresponding ether; a Grignard reagent reacts with a benzyl compound 11 to provide 15. One skilled in the art will appreciate that, while various phenyl compounds are shown in the scheme, the transformations depicted may be applied to similarly substituted indanyl amines. Furthermore, these reactions may also be used to convert a suitable compound 3 or a compound of formula (I) (e.g., a halosubstituted 3 or a halosubstituted formula (I)) to another corresponding compound (e.g., one having an alkyl or cycloalkyl substituent). In some cases, it may be advantageous to protect a reactive amino group; for this purpose a conventional protecting group such as tBoc can be introduced, and later removed using well known synthetic methodologies.

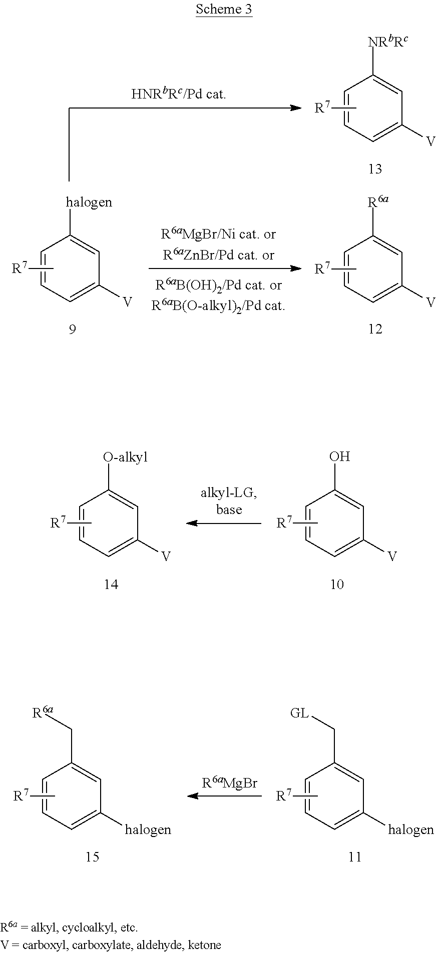

### Biological Function

The utility of the compounds of the present disclosure can be demonstrated by one or more of the following methods or other methods known in the art:
Preparation of ATX, LPC, 4-AAP, TOOS, HRP, and CO
Autotaxin enzyme (ATX, 25 μg) (Echelon Biosciences, Inc. Cat #E-4000, Salt Lake City, Utah) was resuspended in 250 μL sterile water for a 100 μg/mL stock solution.
Lyso PC 14:0 (LPC, Avanti Polar Lipids, Alabaster, Ala.) (200 mg) was resuspended in 7.13 mL sterile water to obtain a 60 mM stock solution.
4-Aminoantipyrine (4-AAP, Sigma-Aldrich, St. Louis, Mo.) was resuspended in 50 mM Tris-HCl, pH 8.0 to obtain a 50 mM stock solution.
3-(N-Ethyl-3-methylanilino)-2-hydroxypropanesulfonic acid sodium salt (TOOS, Sigma-Aldrich) was resuspended in 50 mM Tris-HCl, pH 8.0 to obtain a 30 mM stock solution.
Horseradish peroxidase (HRP, Sigma-Aldrich) was resuspended in 50 mM Tris-HCl, pH 8.0 to obtain a 530 U/mL stock solution.
Choline oxidase (CO, Sigma-Aldrich) (500 U) was resuspended in 2.5 mL 50 mM Tris-HCl, pH 8.0 for a 200 U/mL stock solution.

### Assay Working Solutions Preparation:

Assay buffer: 100 mM Tris-HCl, pH 9.0, 500 mM NaCl, 5 mM MgCl₂, 30 μM CoCl₂, 0.05% Triton X-100
Assay buffer II: 4.5 mM 4-AAP, 2.7 mM TOOS, 21.2 U/mL HRP, 3 U/mL CO, In 50 mM Tris-HCl pH 8.0, 4.5 mM MgCl₂

### Preparation of Test Compounds:

A compound preparation plate (Nunc 249944) was prepared by adding 30 mM stock solution (in DMSO) of test compound and performing a serial full-log dilution down to 30 nM. Two μL/well were transferred from the first compound prep plate to a compound dilution plate (Corning, 3641) and 198 μL assay buffer I was added for a 10× solution.

### In Vitro ATX Assay:

A 10 ng/mL solution of ATX was prepared by diluting in assay buffer I, and 80 μL/well were added to the assay plate (Corning, 3641); negative control wells contained 80 μL/well assay buffer I only (no ATX). Ten μL/well from the compound dilution plate (containing serially diluted test compound or DMSO) were added to the assay plate, which was then incubated at 37° C. for 15 minutes. A solution of LPC 14:0 was prepared in assay buffer I at 3 mM and 10 μl/well were added to the assay plate, for a final concentration of 0.3 mM LPC, and returned to 37° C. incubator for 4 hours. After 4 hour incubation, 100 μL/well of assay buffer II was added to all wells of the assay plate. The plate was incubated at RT for 15 minutes, prior to reading on FlexStation in absorbance mode, 555 nm. Results in the enzymatic ATX inhibition assay are provided for compounds of formula (I). An IC₅₀ value of “A” is less than 100 nM, “B” is equal to or greater than 100 nM but less than 1 μM, “C” is equal to or greater than 1 μM.

TABLE 1

| | ATX |
|---|---|
| Ex. No. | IC₅₀ [μM] |
| 1 | A |
| 2 | B |
| 3 | C |
| 4 | A |
| 5 | B |
| 6 | A |
| 7 | B |
| 8 | B |
| 9 | B |
| 10 | B |
| 11 | C |
| 12 | B |
| 13 | C |
| 14 | C |
| 15 | C |
| 16 | C |
| 17 | C |
| 18 | C |
| 19 | B |
| 20 | C |
| 21 | A |
| 22 | B |
| 23 | A |
| 24 | B |
| 25 | A |
| 26 | B |
| 27 | A |
| 28 | A |
| 29 | A |
| 30 | B |
| 31 | A |
| 32 | B |
| 33 | A |
| 34 | B |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | B |
| 51 | C |
| 52 | A |
| 53 | A |
| 54 | B |
| 55 | B |
| 56 | A |
| 57 | A |
| 58 | A |
| 59 | A |
| 60 | A |
| 61 | B |
| 62 | A |
| 63 | B |
| 64 | A |
| 65 | A |
| 66 | A |
| 67 | A |
| 68 | A |
| 69 | A |
| 70 | A |
| 71 | B |
| 72 | C |
| 73 | C |
| 74 | B |
| 75 | A |
| 76 | A |
| 77 | A |
| 78 | B |
| 79 | A |
| 80 | A |
| 81 | A |
| 82 | B |
| 83 | B |
| 84 | B |
| 85 | A |
| 86 | B |
| 87 | B |
| 88 | B |
| 89 | A |
| 90 | B |
| 91 | B |
| 92 | A |
| 93 | A |
| 94 | B |
| 95 | B |
| 96 | A |
| 97 | A |
| 98 | A |
| 99 | B |
| 100 | B |
| 101 | A |
| 102 | B |
| 103 | A |
| 104 | A |
| 105 | B |
| 106 | B |
| 107 | A |
| 108 | A |
| 109 | A |
| 110 | B |
| 111 | A |
| 112 | A |
| 113 | A |
| 114 | B |
| 115 | A |
| 116 | A |
| 117 | A |
| 118 | A |
| 119 | C |
| 120 | A |
| 121 | A |
| 122 | A |
| 123 | A |
| 124 | B |
| 125 | A |
| 126 | B |
| 127 | B |
| 128 | C |
| 129 | A |
| 130 | B |
| 131 | A |
| 132 | A |
| 133 | A |
| 134 | A |
| 135 | A |
| 136 | A |
| 137 | C |
| 138 | C |
| 139 | B |
| 140 | C |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | B |
| 162 | B |

Compounds of formula (I) and their pharmaceutically acceptable salts or esters thereof as described herein have IC₅₀ values between 0.00001 μM and 1000 μM, particular compounds have IC₅₀ values between 0.0005 μM and 500 μM, further particular compounds have IC₅₀ values between 0.0005 μM and 50 μM, more particular compounds have IC₅₀ values between 0.0005 μM and 5 μM. These results have been obtained by using the enzymatic assay described above.

The disclosure is illustrated hereinafter by Examples, which have no limiting character. In case the preparative examples are obtained as a mixture of enantiomers, the pure enantiomers can be obtained by methods described herein or by methods known to those skilled in the art, such as e.g. chiral chromatography or crystallization.

### Abbreviations

The following abbreviations may be referred to in the experimentals and schemes in the disclosure:

| | |
|---|---|
| Å | Angstrom |
| Ac | Acetate/acetic |
| ACN/MeCN | Acetonitrile |
| aq. | Aqueous |
| Boc | t-Butoxy carbonyl |
| ° C. | Degrees Celsius |
| DCM | Dichloromethane |
| DIPEA | Diisopropyl ethylamine |
| DMF | Dimethylformamide |
| DMSO | Dimethyl sulfoxide |
| ee | Enantiomeric excess |
| Eq. | Equivalent(s) |
| ESI | Electrospray ionization |
| EtOAc | Ethyl acetate |
| EtOH | Ethanol |
| h | Hour(s) |
| HATU | 1-[Bis(dimethylamino)methylene]-1H-1,2,3- |
| | triazolo[4,5-b]pyridinium 3-oxide hexafluorophosphate |
| Hex | Hexanes |
| LC-MS | Liquid chromatography/mass spectrometry (Shimadzu, |
| | Model#: LCMS-2020) |
| M | Molar |
| min | Minute(s) |
| MS | Mass spectrometry |
| N | Normal |
| Pd(dppf)Cl₂ | [1,1′- |
| | Bis(diphenylphosphino)ferrocene]dichloropalladium(II) |
| psi | Pounds per square inch |
| RT | Room temperature |
| SFC | Supercritical fluid chromatography |
| TFA | Trifluoracetic acid |
| THF | Tetrahydrofuran |
| v | Volume |
| w/w | Weight per weight |
| Xantphos | 4,5-Bis(diphenylphosphino)-9,9-dimethylxanthene |

Alkyl abbreviations include:
Me (methyl),
Et (ethyl),
Pr (propyl),
iPr (isopropyl),
Bu (butyl),
sBu (sec-butyl),
tBu (tertiary butyl).

### EXAMPLES

All examples and intermediates were prepared under nitrogen atmosphere if not specified otherwise.

Intermediates A1-A26: Intermediates AT-A26 are commercially available and listed in the following table:

| Int. | Structure | Systemic Name |
|---|---|---|
| A1 | 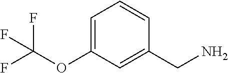 [SMILES: NCc1cccc(OC(F)(F)F)c1] | (3-(trifluoro- methoxy)phenyl)- methanamine |
| A2 | 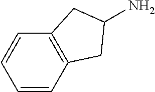 | 2,3-dihydro-1H- inden-2-amine |
| A3 | 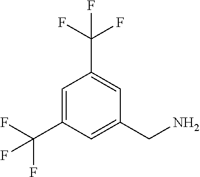 [SMILES: NCc1cc(C(F)(F)F)cc(C(F)(F)F)c1] | (3,5-bis(trifluoro- methyl)phenyl)- methanamine |
| A4 | 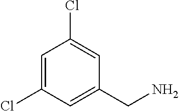 | (3,5-dichlorophenyl)- methanamine |
| A5 | 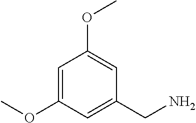 | (3,5-dimethoxy- phenyl)methanamine |
| A6 | 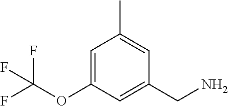 | (3-methyl-5- (trifluoromethoxy)phe- nyl)methanamine |
| A7 | 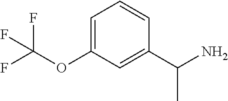 | 1-(3-(trifluroo- methoxy)phenyl)- ethanamine |
| A8 | 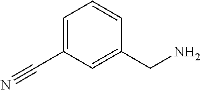 | 3-cyanobenzylamine |
| A9 | 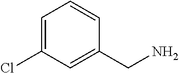 | (3-chlorophenyl)- methanamine |
| A10 | 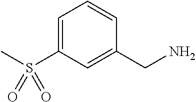 | (3-(methylsulfonyl)- phenyl)methanamine |
| A11 | 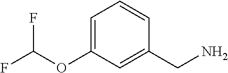 [SMILES: NCc1cccc(OC(F)F)c1] | (3-(difluoro- methoxy)phenyl)- methanamine |
| A12 | 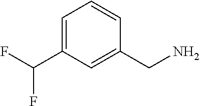 [SMILES: NCc1cccc(C(F)F)c1] | (3-(difluoromethyl)- phenyl)methanamine |
| A13 | 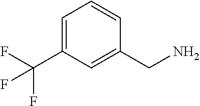 [SMILES: NCc1cccc(C(F)(F)F)c1] | (3-(trifluoromethyl)- phenyl)methanamine |
| A14 | 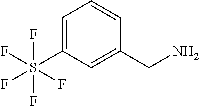 [SMILES: NCc1cccc(S(F)(F)(F)(F)F)c1] | (3-(pentafluorothio)- phenyl)methanamine |
| A15 | 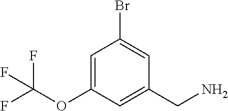 [SMILES: NCc1cc(Br)cc(OC(F)(F)F)c1] | (3-bromo-5- (trifluoromethoxy)- phenyl)methanamine |
| A16 | 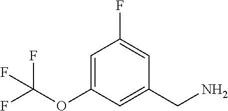 [SMILES: NCc1cc(F)cc(OC(F)(F)F)c1] | (3-fluoro-5- (trifluoromethoxy)- phenyl)methanamine |
| A17 | 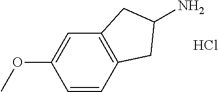 | 5-methoxy-2,3- dihydro-1H-inden-2- amine hydrochloride |
| A18 | 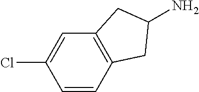 [SMILES: NC1Cc2ccc(Cl)cc2C1] | 5-chloro-2,3- dihydro-1H-inden-2- amine |
| A19 | 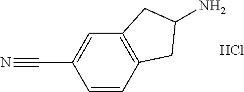 [SMILES: N#Cc1ccc2c(c1)CC(N)C2 ; Cl] | 2-amino-2,3- dihydro-1H-indene- 5-carbonitrile HCl |
| A20 | 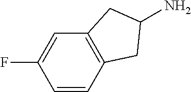 [SMILES: NC1Cc2ccc(F)cc2C1] | 5-fluoro-2,3-dihydro- 1H-inden-2-amine |
| A21 | 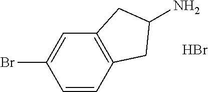 [SMILES: NC1Cc2ccc(Br)cc2C1 ; Br] | 5-bromo-2,3- dihydro-1H-inden-2- amine hydrobromide |
| A22 | 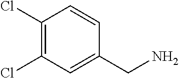 | (3,4-dichlorophenyl)- methanamine |
| A23 | 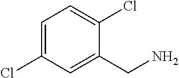 | 2,5-dichlorobenzyl- amine |
| A24 | 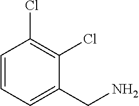 | 2,3-dichlorobenzyl- amine |
| A25 | 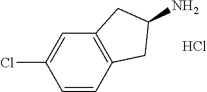 [SMILES: N[C@H]1Cc2ccc(Cl)cc2C1 ; Cl] | (S)-5-bromo-2,3- dihydro-1H-inden-2- amine HCl |
| A26 | 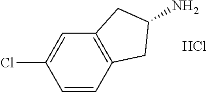 [SMILES: N[C@@H]1Cc2ccc(Cl)cc2C1 ; Cl] | (R)-5-bromo-2,3- dihydro-1H-inden-2- amine HCl |

### Intermediate A27: (3-(tert-Butyl)-5-(trifluoromethoxy)phenyl)methanamine hydrochloride

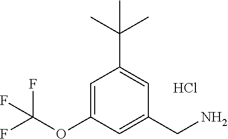 [SMILES: CC(C)(C)c1cc(CN)cc(OC(F)(F)F)c1 ; Cl]

### Step 1: Ethyl 3-bromo-5-(trifluoromethoxy)benzoate

To a solution of 3-bromo-5-(trifluoromethoxy)benzoic acid (2.0 g, 7.0 mmol 1.0 eq.) in EtOH (20 mL) was added SOCl₂ (1.0 mL, 14 mmol, 2.0 eq.). After stirring for 17 h at RT, the mixture was partitioned between EtOAc and aq. NaHCO₃. The aqueous phase was separated and washed once with EtOAc. The combined organics were washed with brine, dried over MgSO₄, filtered and concentrated. The residue was purified on SiO₂ to give ethyl 3-bromo-5-(trifluoromethoxy)benzoate (1.9 g, 85% yield).

### Step 2: Ethyl 3-(tert-butyl)-5-(trifluoromethoxy)benzoate

Ethyl 3-bromo-5-(trifluoromethoxy)benzoate (0.9 g, 2.9 mmol, 1.0 eq.) was dissolved in THF (5.8 mL, inhibitor free). Argon gas was bubbled through the solution for 10 minutes. 1,3-Dicyclohexyl-1H-imidazol-3-ium tetrafluoroborate (92 mg, 0.29 mmol, 10 mol %) and NiCl₂.1.5 water (45 mg, 0.29 mmol, 10 mol %, see J. Am. Chem. Soc. 2011, 133, 8478-8481) were added and the mixture was cooled to −10° C. A 1 M solution of tert-butylmagnesium chloride in THF (5.8 mL, 5.8 mmol, 2.0 eq.) was added dropwise, maintaining the temperature of the mixture below −8° C. After 1 h, the mixture was partitioned between EtOAc and 10% aq. NH₄Cl. The aqueous phase was separated and washed with EtOAc. The combined organic extracts were washed with brine, dried over MgSO₄, filtered and concentrated. The residue was purified on SiO₂ to give ethyl 3-(tert-butyl)-5-(trifluoromethoxy)benzoate (0.68 g, 81% yield).

### Step 3: (3-(tert-Butyl)-5-(trifluoromethoxy)phenyl)methanol

To a solution of ethyl 3-(tert-butyl)-5-(trifluoromethoxy)benzoate (0.68 g, 2.3 mmol, 1.0 eq.) in THF (5 mL) was added a 1M solution of LiBH₄ in THF (5.8 mL, 12 mmol, 5.0 eq.) and the mixture was heated to 50° C. for 5.5 h. The mixture was quenched with 1 M HCl and extracted with EtOAc. The organic phase was separated, washed with water then brine, dried over MgSO₄, filtered and concentrated to give (3-(tert-butyl)-5-(trifluoromethoxy)phenyl)methanol (0.54 g, 94% yield) which was used without further purification.

### Step 4: 3-(tert-Butyl)-5-(trifluoromethoxy)benzaldehyde

To a solution of (3-(tert-butyl)-5-(trifluoromethoxy)phenyl)methanol (0.54 g, 2.2 mmol, 1.0 eq.) in CH₂Cl₂ was added Dess-Martin periodinane (1.0 g, 2.4 mmol, 1.1 eq.). After 4 h, the mixture was quenched with Na₂S₂O₃ then partitioned between EtOAc and saturated aq. NaHCO₃. After filtration to removed solids, the organic phase was separated, washed with brine, dried over MgSO₄, filtered and concentrated. The residue was purified on SiO₂ to give 3-(tert-butyl)-5-(trifluoromethoxy)benzaldehyde (0.34 g, 64% yield).

### Step 5: (3-(tert-Butyl)-5-(trifluoromethoxy)phenyl)methanamine hydrochloride

To 3-(tert-butyl)-5-(trifluoromethoxy)benzaldehyde (0.34 g, 1.4 mmol, 1.0 eq.) in EtOH (9 mL) at RT was added HONH₂.HCl (0.15 g, 2.1 mmol, 1.5 eq.). After 16 h, conc. HCl (0.58 mL, 7.0 mmol, 5 eq.) and 20% Pd(OH)₂ on carbon (0.2 g) were added and the mixture was placed on a Parr shaker at 65 psi for 1 h. The mixture was filtered through a pad of celite then concentrated to give (3-(tert-butyl)-5-(trifluoromethoxy)phenyl)methanamine hydrochloride, which was used without further purification. MS (ESI): m/z=248 [M+H]⁺

### Intermediate A28: (3-Cyclobutyl-5-(trifluoromethoxy)phenyl)methanamine hydrochloride

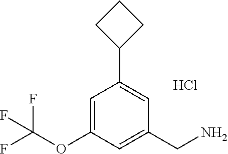 [SMILES: NCc1cc(OC(F)(F)F)cc(C2CCC2)c1 ; Cl]

### Step 1: Ethyl 3-cyclobutyl-5-(trifluoromethoxy)benzoate

A solution of ethyl 3-bromo-5-(trifluoromethoxy)benzoate (0.89 g, 2.9 mmol, 1.0 eq., product of step 1 of intermediate A27) in THF (inhibitor-free, 10 mL) was purged with N₂ for 10 minutes. Cyclobutylzinc bromide (8.5 mL, 4.3 mmol, 1.5 eq., 0.5 M in THF) and bis(tri-tert-butylphosphine)palladium (0.15 g, 0.29 mmol, 10 mol %) were added and the mixture was heated to 45° C. for 22 h. After cooling, the mixture was partitioned between EtOAc and aq. NH₄Cl. The organic phase was separated, washed with brine, dried over MgSO₄, filtered and concentrated. The residue was purified on SiO₂ to give ethyl 3-cyclobutyl-5-(trifluoromethoxy)benzoate (0.44 g, 53% yield).

### Step 2: (3-Cyclobutyl-5-(trifluoromethoxy)phenyl)methanamine hydrochloride

The title compound was prepared in analogy to Steps 3-5 of A27 using ethyl 3-cyclobutyl-5-(trifluoromethoxy)benzoate. MS (ESI): m/z=246 [M+H]⁺

### Intermediate A29: (3-Isopropoxy-5-(trifluoromethoxy)phenyl)methanamine

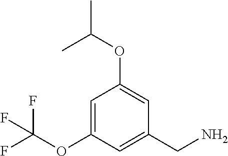 [SMILES: CC(C)Oc1cc(CN)cc(OC(F)(F)F)c1]

### Step 1: 3-Isopropoxy-5-(trifluoromethoxy)benzaldehyde

To a solution of 3-hydroxy-5-(trifluoromethoxy)benzaldehyde (0.40 g, 1.9 mmol, 1.0 eq.) in DMF (2 mL) was added 2-iodopropane (0.33 mL, 2.9 mmol, 1.5 eq.) and K₂CO₃ (0.67 g, 4.9 mmol, 2.5 eq.). The mixture was heated to 70° C. for 20 h, then cooled to RT and partitioned between EtOAc and water. The organic layer was separated, washed with brine, dried over MgSO₄, filtered and concentrated. The residue was purified on SiO₂ to give 3-isopropoxy-5-(trifluoromethoxy)benzaldehyde (0.30 g, 62% yield).

### Step 2: (3-Isopropoxy-5-(trifluoromethoxy)phenyl)methanamine

To 3-isopropoxy-5-(trifluoromethoxy)benzaldehyde (0.3 g, 1.2 mmol, 1.0 eq.) in EtOH (5 mL) at RT was added HONH₂.HCl (0.13 g, 1.8 mmol, 1.5 eq.). After 30 min, conc. HCl (0.50 mL, 6.0 mmol, 5.0 eq.) and 20% Pd(OH)₂ on carbon (0.2 g) were added and the mixture was placed under H₂ atmosphere at 50° C. for 18 h. The mixture was cooled, filtered through a pad of celite and concentrated. The residue was purified on a reverse phase C₁₈ column to give (3-isopropoxy-5-(trifluoromethoxy)phenyl)methanamine (0.15 g, 50% yield). MS (ESI): m/z=250 [M+H]⁺

Intermediate A30-A35: The following intermediates were prepared in analogy to intermediate A29 using the indicated starting material(s) in step 1.

| | | | MS |
|---|---|---|---|
| | | Starting | (ESI): |
| Int. | Structure/Name | Material | m/z |
| A30 | 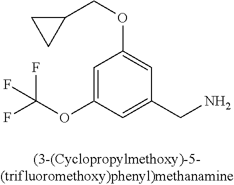 [SMILES: NCc1cc(OCC2CC2)cc(OC(F)(F)F)c1] | (bromomethyl)- cyclopropane | 262 [M + H]⁺ |
| A31 | 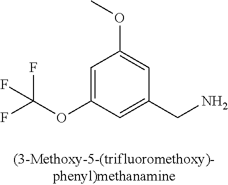 | indomethane | 222 [M + H]⁺ |
| A32 | 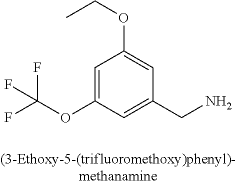 [SMILES: CCOc1cc(CN)cc(OC(F)(F)F)c1] | iodoethane | 236 [M + H]⁺ |
| A33 | 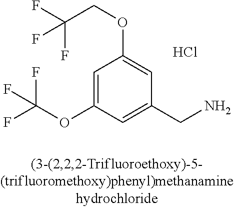 [SMILES: NCc1cc(OCC(F)(F)F)cc(OC(F)(F)F)c1 ; Cl] | 1,1,1-trifluoro-2- iodoethane | 290 [M + H]⁺ |
| A34 | 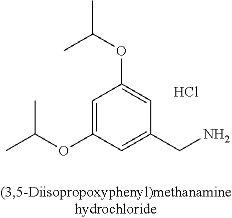 [SMILES: CC(C)Oc1cc(CN)cc(OC(C)C)c1 ; Cl] | 3,5-dihydroxy- benzaldehyde and 3 eq. of 2-iodopropane | 224 [M + H]⁺ |
| A35 | 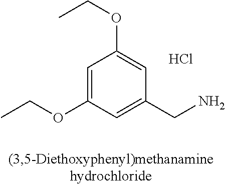 | 3,5-dihydroxy- benzaldehyde and 3 eq. of iodoethane | 196 [M + H]⁺ |

### Intermediate A36: (3-(Cyclopropylmethyl)-5-(trifluoromethoxy)phenyl)methanamine

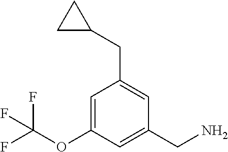 [SMILES: NCc1cc(CC2CC2)cc(OC(F)(F)F)c1]

### Step 1: 1-Bromo-3-(cyclopropylmethyl)-5-(trifluoromethoxy)benzene

To a solution of cyclopropylmagnesium bromide (18 mL, 9 mmol, 1.5 eq., 0.5 M in THF) at 0° C. was added CuI (0.11 g, 0.6 mmol, 10 mol %). After stirring for 15 minutes, a solution of 1-bromo-3-(bromomethyl)-5-(trifluoromethoxy)benzene (2.0 g, 6.0 mmol, 1.0 eq.) in THF (10 mL) was added dropwise via addition funnel. The mixture was allowed to warm to RT and stirred for 3 h. In a separate flask, CuI (0.05 g, 0.3 mmol, 5 mol %) was added to a solution of cyclopropylmagnesium bromide (9 mL, 4.5 mmol, 0.75 eq., 0.5 M in THF) at 0° C. After stirring 15 minutes, the freshly prepared reagent was added to the mixture and stirred for 18 h. The mixture was quenched with aq. NH₄Cl then extracted with EtOAc and filtered through a pad of celite. The filtrate was dried over MgSO₄, filtered, concentrated, and then purified on SiO₂ to give 1-bromo-3-(cyclopropylmethyl)-5-(trifluoromethoxy)benzene (1.2 g, 70% yield).

### Step 2: 3-(Cyclopropylmethyl)-5-(trifluoromethoxy)benzaldehyde

To a solution of 1-bromo-3-(cyclopropylmethyl)-5-(trifluoromethoxy)benzene (1.2 g, 4.1 mmol, 1.0 eq.) in THF at −78° C. was added n-butyllithium (1.9 mL, 1.1 eq., 2.5 M in hexane), maintaining the temperature of the mixture below −70° C. Once the addition was complete, the mixture was stirred for 15 minutes and then DMF (0.49 mL, 6.1 mmol, 1.5 eq.) was added, again maintaining the temperature of the mixture below −70. The mixture was quenched with MeOH and partitioned between EtOAc and aq. NH₄Cl. The organic layer was separated, washed with brine, dried over MgSO₄, filtered, concentrated, and purified on SiO₂ to give 3-(cyclopropylmethyl)-5-(trifluoromethoxy)benzaldehyde (0.77 g, 74% yield).

### Step 3: (3-(Cyclopropylmethyl)-5-(trifluoromethoxy)phenyl)methanamine

The title compound was prepared using 3-(cyclopropylmethyl)-5-(trifluoromethoxy)benzaldehyde in analogy to Step 2/A29. MS (ESI): m/z=246 [M+H]⁺

### Intermediate A37: (3-Isopropyl-5-(trifluoromethoxy)phenyl)methanamine hydrochloride

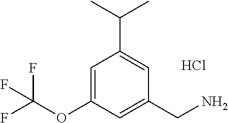 [SMILES: CC(C)c1cc(CN)cc(OC(F)(F)F)c1 ; Cl]

### Step 1: 3-(Prop-1-en-2-yl)-5-(trifluoromethoxy)benzaldehyde

To 3-bromo-5-(trifluoromethoxy)benzaldehyde (15 g, 56 mmol, 1.0 eq.) in dioxane (112 mL) and water (38 mL) was added 4,4,5,5-tetramethyl-2-(prop-1-en-2-yl)-1,3,2-dioxaborolane (14 g, 84 mmol, 1.5 eq.) and K₂CO₃ (23 g, 170 mmol, 3.0 eq.). N₂ was bubbled through the mixture for 15 minutes. Pd(PPh₃)₄ (3.2 g, 2.8 mmol, 5 mol %) was added and the reaction vessel was sealed and heated to 85° C. for 20 h. The mixture was cooled to RT then partitioned between EtOAc and water. The aqueous layer was extracted once with EtOAc. The organic layers were combined, dried over MgSO₄, filtered and concentrated. The residue was purified on SiO₂ to give 3-(prop-1-en-2-yl)-5-(trifluoromethoxy)benzaldehyde (11 g, 85% yield).

### Step 2: (3-Isopropyl-5-(trifluoromethoxy)phenyl)methanamine hydrochloride

To 3-(prop-1-en-2-yl)-5-(trifluoromethoxy)benzaldehyde (11 g, 47 mmol, 1.0 eq.) in EtOH (165 mL) at RT was added HONH₂.HCl (4.9 g, 71 mmol, 1.5 eq.). After 18 h, conc. HCl (8.0 mL, 95 mmol, 2.0 eq.) and 20% Pd(OH)₂ on carbon (2.2 g) were added and the mixture was placed on a Parr shaker under 60 psi of hydrogen for 18 h. An additional charge of HCl (4 mL, 47 mmol, 1.0 eq.) and 20% Pd(OH)₂ on carbon (1.3 g) were added and the mixture was replaced under hydrogen on a Parr shaker at 60 psi for 24 h. The mixture was filtered through a pad of celite and the filtrate was concentrated. The residue was dissolved in EtOAc, washed twice with saturated NaHCO₃, dried over MgSO₄, filtered, concentrated and purified on SiO₂. To a solution of the crude mixture (5.4 g) in EtOH (80 mL) was added HCl (4 mL) and 20% Pd(OH)₂ on carbon (1.5 g). The mixture was placed on a Parr shaker at 60 psi of hydrogen for 18 h. The mixture was filtered through a pad of celite and the filtrate was concentrated to give (3-isopropyl-5-(trifluoromethoxy)phenyl)methanamine hydrochloride (6.2 g, 49% yield). MS (ESI): m/z=234 [M+H]⁺

### Intermediate A38: (3-Isopropyl-5-(pentafluorothio)phenyl)methanamine

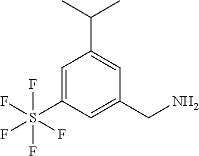 [SMILES: CC(C)c1cc(CN)cc(S(F)(F)(F)(F)F)c1]

The title compound was prepared in analogy to A37 using 3-bromo-5-(pentafluorothio)benzaldehyde in Step 1. MS (ESI): m/z=276 [M+H]⁺

### Intermediate A39: (3-Cyclopropyl-5-(trifluoromethoxy)phenyl)methanamine

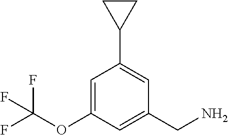 [SMILES: NCc1cc(OC(F)(F)F)cc(C2CC2)c1]

### Step 1: 3-Cyclopropyl-5-(trifluoromethoxy)benzaldehyde

To 3-bromo-5-(trifluoromethoxy)benzaldehyde (2 g, 7.4 mmol, 1.0 eq.) in dioxane (20 mL) was added cyclopropylboronic acid (1.3 g, 15 mmol, 2.0 eq.) and cesium fluoride (3.8 g, 25 mmol, 3.4 eq.). N₂ was bubbled through the mixture for 10 minutes. PdCl₂(dppf).CH₂Cl₂ (0.60 g, 0.74 mmol, 10 mol %) was added and the reaction vessel was sealed and heated to 90° C. for 18 h. The mixture was partitioned between EtOAc and water and filtered through a pad of celite. The organic layer was separated, washed with brine, dried over MgSO₄, filtered and concentrated. The residue was purified on SiO₂ to give 3-cyclopropyl-5-(trifluoromethoxy)benzaldehyde (1.6 g, 93% yield).

### Step 2: (3-Cyclopropyl-5-(trifluoromethoxy)phenyl)methanamine

To 3-cyclopropyl-5-(trifluoromethoxy)benzaldehyde (0.94 g, 4.1 mmol, 1.0 eq.) in EtOH (9 mL) at RT was added HONH₂.HCl (0.43 g, 6.2 mmol, 1.5 eq.). After 1.5 h, conc. HCl (0.85 mL, 10 mmol, 2.5 eq.) and 10% palladium on carbon (0.2 g, 20 wt %) were added and the mixture was stirred under hydrogen atmosphere for 22 h. An additional charge of HCl was added (0.85 mL, 10.3 mmol, 2.5 eq.) and the mixture was placed on a Parr shaker at 60 psi for 24 h. The mixture was filtered through a pad of celite, concentrated, and purified on a reverse phase C₁₈ column to give (3-cyclopropyl-5-(trifluoromethoxy)phenyl)methanamine (0.87 g, 84% yield) as a formate salt. MS (ESI): m/z=232 [M+H]⁺

### Intermediate A40 (3-Cyclopropyl-5-(trifluoromethyl)phenyl)methanamine

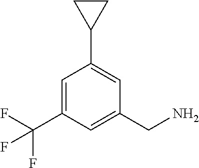 [SMILES: NCc1cc(C2CC2)cc(C(F)(F)F)c1]

The title compound was prepared in analogy to A39 using 3-bromo-5-(trifluoromethyl)benzaldehyde in Step 1. MS (ESI): m/z=216 [M+H]⁺

### Intermediate A41: (3,5-Dicyclopropyl-phenyl)methanamine

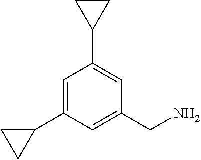 [SMILES: NCc1cc(C2CC2)cc(C2CC2)c1]

The title compound was prepared in analogy to A39 using 3,5-dibromo-benzaldehyde and 4 eq. of cyclopropylboronic acid in Step 1. MS (ESI): m/z=188 [M+H]⁺

### Intermediate A42: (3-Cyclopropyl-5-isopropoxyphenyl)methanamine

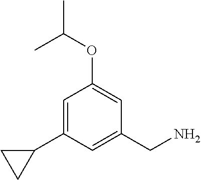

### Step 1: Methyl 3-cyclopropyl-5-isopropoxybenzoate

The title compound was prepared in analogy to Step 1/A39 using methyl 3-bromo-5-isopropoxybenzoate.

### Step 2: (3-Cyclopropyl-5-isopropoxyphenyl)methanol

The title compound was prepared in analogy to Step 3/A27.

### Step 3: 3-Cyclopropyl-5-isopropoxybenzaldehyde

The title compound was prepared in analogy to Step 4/A27.

### Step 4: (3-Cyclopropyl-5-isopropoxyphenyl)methanamine

The title compound was prepared in analogy to Step 2/A29 to give (3-cyclopropyl-5-isopropoxyphenyl)methanamine after purification on reverse phase C₁₈ column. MS (ESI): m/z=206 [M+H]⁺

### Intermediate A43: (3-Isopropoxy-5-propylphenyl)methanamine

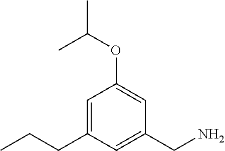

(3-Isopropoxy-5-propylphenyl)methanamine was isolated as a byproduct from step 4 of intermediate A42. MS (ESI): m/z=208 [M+H]⁺

### Intermediate A44: (3-Cyclopropyl-5-ethoxyphenyl)methanamine

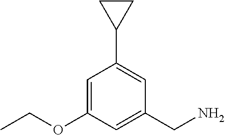

The title compound was prepared in analogy to A42 using methyl 3-bromo-5-ethoxybenzoate in Step 1. MS (ESI): m/z=192 [M+H]⁺

### Intermediate A45: 3-Bromo-5-isopropoxyphenyl)methanamine

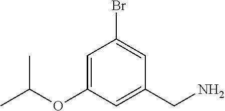

### Step 1: 3-Bromo-5-isopropoxybenzamide

To methyl 3-bromo-5-isopropoxybenzoate (1.1 g, 3.9 mmol, 1.0 eq.), was added 15 mL of 7 N ammonia in MeOH. The mixture was heated to 50° C. in pressure vessel for 16 h. An additional 10 mL of 7 N ammonia in MeOH was added and heating was continued at 50° C. for 16 h. The mixture was concentrated to remove solvent the residue was purified on SiO₂ to give 3-bromo-5-isopropoxybenzamide (0.92 g, 92% yield).

### Step 2: (3-Bromo-5-isopropoxyphenyl)methanamine

Lithium aluminum hydride (2.7 mL, 5.4 mmol, 1.5 eq., 2.0 M in THF) was added to a solution of 3-bromo-5-isopropoxybenzamide (0.90 g, 3.5 mmol, 1.0 eq.) in 10 mL THF at 0° C. The ice bath was removed and the mixture was heated to 70° C. for 1.5 h. The mixture was then partitioned between EtOAc and water. The aqueous layer was extracted twice more with EtOAc and the combined organic layers concentrated. The residue was purified on a reverse phase C₁₈ column to isolate (3-bromo-5-isopropoxyphenyl)methanamine (0.26 g, 30% yield). MS (ESI): m/z=244 [M+H]⁺

### Intermediate A46: (3-(Azetidin-1-yl)-5-isopropoxyphenyl)methanamine

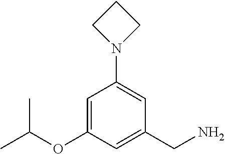 [SMILES: CC(C)Oc1cc(CN)cc(N2CCC2)c1]

### Step 1: Methyl 3-(azetidin-1-yl)-5-isopropoxybenzoate

To methyl 3-bromo-5-isopropoxybenzoate (1.4 g, 4.7 mmol, 1.0 eq.) in toluene (14 mL) was added azetidine hydrochloride (0.88 g, 9.4 mmol, 2 eq.) and Cs₂CO₃ (6.1 g, 19 mmol, 4.0 eq.). The mixture was subsurface purged for 10 minutes with nitrogen. Pd(OAc)₂ (0.063 g, 0.28 mmol, 0.06 eq.) and Xantphos (0.22 g, 0.38 mmol, 0.08 eq.) were added and the mixture was heated to 100° C. for 18 h, then cooled to RT and partitioned between EtOAc and water. The aqueous layer was extracted once with EtOAc and the combined organic layers were dried over MgSO₄, filtered and concentrated. The residue was purified on SiO₂ to give methyl 3-(azetidin-1-yl)-5-isopropoxybenzoate (0.98 g, 84% yield).

### Step 2: 3-(Azetidin-1-yl)-5-isopropoxybenzamide

The title compound was prepared in analogy to Step 1/A45 using methyl 3-(azetidin-1-yl)-5-isopropoxybenzoate.

### Step 3: 3-(Azetidin-1-yl)-5-isopropoxyphenyl)methanamine

To a solution of 3-(azetidin-1-yl)-5-isopropoxybenzamide (0.91 g, 3.6 mmol, 1.0 eq.) in THF (18 mL) at 0° C. was added borane (1.1 mL, 11 mmol, 3 eq., 10 M in dimethyl sulfide). After the addition was complete the mixture was heated 60° C. for 18 h then cooled to RT and quenched carefully with 2-propanol. The volatiles were removed to provide 3-(azetidin-1-yl)-5-isopropoxyphenyl)methanamine (1.1 g, 100% yield) which was used without further purification. MS (ESI): m/z=221 [M+H]⁺

### Intermediate A47: (3-(Azetidin-1-yl)-5-(trifluoromethoxy)phenyl)methanamine

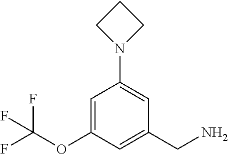 [SMILES: NCc1cc(OC(F)(F)F)cc(N2CCC2)c1]

The title compound was prepared in analogy to A46 using methyl 3-bromo-5-(trifluoromethoxy)benzoate in Step 1. MS (ESI): m/z=247 [M+H]⁺

Intermediates B: B1-B39 listed in the following table are commercially available:

| Int. | Structure | Systemic Name |
|---|---|---|
| B1 | 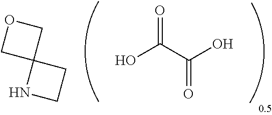 | 6-oxa-1-aza- spiro[3.3]hep- tane hemioxalate |
| B2 | 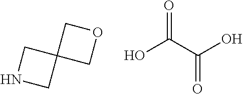 [SMILES: C1NCC12COC2 ; O=C(O)C(=O)O] | 2-oxa-6-aza- spiro[3.3]hep- tane oxalate |
| B3 | 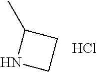 | 2-methyl- azetidine HCl |
| B4 | 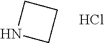 | azetidine HCl |
| B5 | 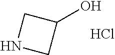 | 3-hydroxy- azetidine HCl |
| B6 | 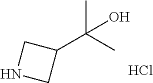 | 2-azetidin-3-yl- propan-2-ol HCl |
| B7 | 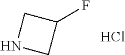 | 3-fluoroazetidine HCl |
| B8 | 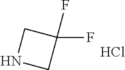 | 3,3-difluoro- azetidine HCl |
| B9 | 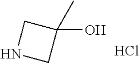 | 3-hydroxy-3- methylazetidine HCl |
| B10 | 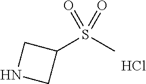 | 3-methyl- sulfonyl- azetidine HCl |
| B11 | 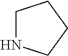 | pyrrolidine |
| B12 | 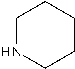 | piperidine |
| B13 | 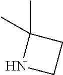 | 2,2-dimethyl- azetidine |
| B14 | 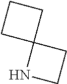 | 1-azaspiro[3.3]- heptane |
| B15 | 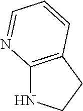 | 2,3-dihydro-1H- pyrrolo[2,3-b]- pyridine |
| B16 | 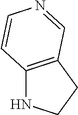 | 2,3-dihydro-1H- pyrrolo[3,2-c]- pyridine |
| B17 | 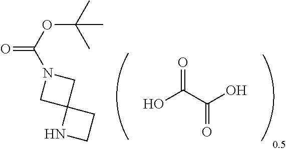 | tert-butyl 1,6- diazaspiro[3.3]- heptane-6- carboxylate hemioxalate |
| B18 | 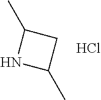 | 2,4-dimethyl- azetidine HCl |
| B19 | 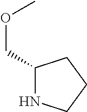 | (S)-2-(methoxy- methyl)- pyrrolidine |
| B20 | 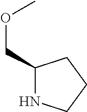 | (R)-2-(methoxy- methyl)- pyrrolidine |
| B21 | 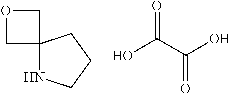 | 2-oxa-5-aza- spiro[3.4]octane oxalate |
| B22 | 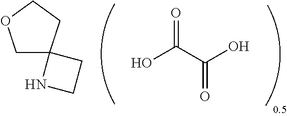 | 6-oxa-1-aza- spiro[3.4]octane hemioxalate |
| B23 | 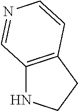 | 2,3-dihydro-1H- pyrrolo[2,3-c]- pyridine |
| B24 | 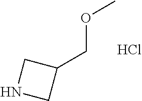 | 3-(methoxy- methyl)azetidine HCl |
| B25 | 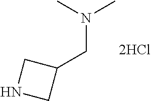 | 1-(azetidin-3-yl)- N,N-dimethyl- methanamine diHCl |
| B26 | 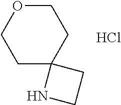 | 7-oxa-1-aza- spiro[3.5]nonane HCl |
| B27 | 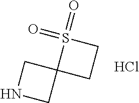 | 1-thia-6-aza- spiro[3.3]hep- tane- 1,1-dioxoide HCl |
| B28 | 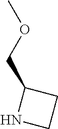 | (2R)-2- (methoxy- methyl)azetidine |
| B29 | 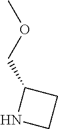 | (2S)-2- (methoxy- methyl)azetidine |
| B30 | 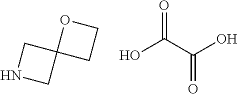 | 1-oxa-6-aza- spiro[3.3]hep- tane oxalate |
| B31 | 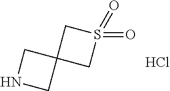 | 2-thia-6-aza- spiro[3.3]heptane 2,2-dioxide HCl |
| B32 | 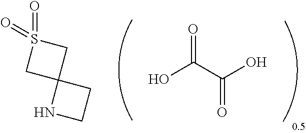 | 2-thia-5-aza- spiro[3.3]heptane- 2,2-dione hemioxalate |
| B33 | 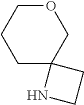 | 6-oxa-1-aza- spiro[3.5]nonane |
| B34 | 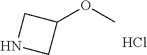 | 3-methoxy- azetidine HCl |
| B35 | 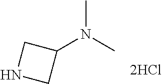 | 3-(dimethyl- amino)azetidine diHCl |
| B36 | 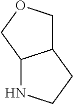 | hexahydro- furo[3,4-b]- pyrrole |
| B37 | 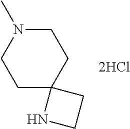 | 7-methyl-1,7- diazaspiro[3.5]no- nane diHCl |
| B38 | 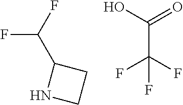 | 2-(difluroo- methyl)azetidine trifluoroacetate |
| B39 | 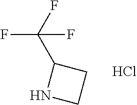 | 2-(trifluoro- methyl)azetidine HCl |
| B40 | 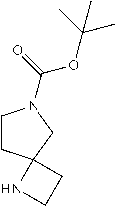 | tert-butyl 1,6- diazaspiro[3,4]- octane-6- carboxylate |
| B41 | 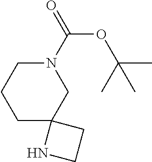 [SMILES: CC(C)(C)OC(=O)N1CCCC2(CCN2)C1] | tert-butyl 1,6- diazaspiro[3.5]- nonane-6- carboxylate |
| B42 | 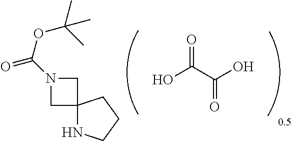 | tert-butyl 2,5- diazaspiro[3.4]- octane-2- carboxylate hemioxalate |

### Intermediate B43: 2-(Azetidin-2-yl)propan-2-ol 2,2,2-trifluoroacetate

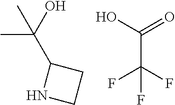

### Step 1: tert-Butyl 2-(2-hydroxypropan-2-yl)azetidine-1-carboxylate

To a solution of methyl 1-Boc-azetidine-2-carboxylate (4.3 g, 20 mmol, 1.0 eq.) in diethyl ether (40 mL) at 0° C. was added MeMgBr (40 mL, 120 mmol, 6.0 eq., 3.0 M in Et₂O) dropwise via addition funnel over 20 minutes. After stirring for 2 h, the mixture was quenched with aqueous NH₄Cl then partitioned between EtOAc and 1 M HCl. The organic layer was separated, washed with water then brine, dried over MgSO₄, filtered and concentrated to give tert-butyl 2-(2-hydroxypropan-2-yl)azetidine-1-carboxylate (3.0 g, 69% yield), which was used without further purification.

### Step 2: 2-(Azetidin-2-yl)propan-2-ol 2,2,2-trifluoroacetate

To a solution of tert-butyl 2-(2-hydroxypropan-2-yl)azetidine-1-carboxylate (3.0 g, 14 mmol, 1.0 eq.) in DCM (30 mL) was added trifluoroacetic acid (10 mL, 140 mmol, 10 eq.). After 3 h, the mixture was concentrated under vacuum to give 2-(azetidin-2-yl)propan-2-ol 2,2,2-trifluoroacetate (4.5 g, 94% yield), which was used without further purification. MS (ESI): m/z=116 [M+H]⁺

### Intermediate B44-B46

The following intermediates were prepared in analogy to B43 using the indicated starting material in Step 1.

| | | | MS (ESI): |
|---|---|---|---|
| Int. | Structure/Name | Starting Material | m/z |
| B44 | 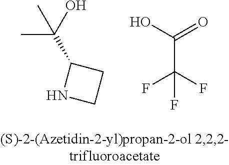 | (S)-1-Boc-azetidine-2- carboxylic acid methyl ester | 116 [M + H]⁺ |
| B45 | 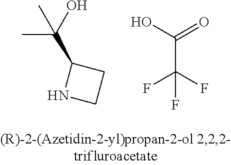 | (R)-1-Boc-azetidine-2- carboxylic acid methyl ester | 116 [M + H]⁺ |
| B46 | 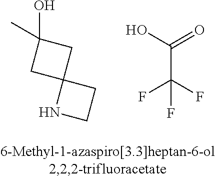 | tert-butyl 6-oxo-1- azaspiro[3.3]heptane-1- carboxylate | 128 [M + H]⁺ |

### EXAMPLES

### Example 1. (2-((3-Isopropyl-5-(trifluoromethoxy)benzyl)amino)pyrimidin-5-yl) (6-oxa-1-azaspiro[3.3]heptan-1-yl)methanone

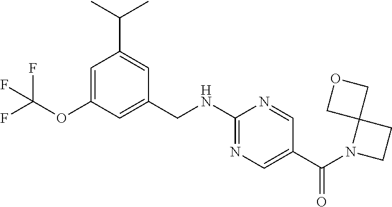 [SMILES: CC(C)c1cc(CNc2ncc(C(=O)N3CCC34COC4)cn2)cc(OC(F)(F)F)c1]

### Procedure A: SNAr with 2-chloropyrimidine-5-carboxylic acid followed by amide coupling Step 1: 2-((3-Isopropyl-5-(trifluoromethoxy)benzyl)amino)pyrimidine-5-carboxylic acid

To a solution of (3-isopropyl-5-(trifluoromethoxy)phenyl)methanamine hydrochloride (intermediate A37, 2.8 g, 10 mmol, 1.0 eq.) in N-methyl-2-pyrrolidone (25 mL) was added 2-chloropyrimidine-5-carboxylic acid (1.7 g, 10 mmol, 1.0 eq.) and K₂CO₃ (7.2 g, 52 mmol, 5.0 eq.) and the mixture was heated to 120° C. for 24 h. After cooling to RT, conc. HCl (8.4 mL) and water were added and the resulting suspension was stirred for 18 h. After filtration, the crude solids were suspended in EtOAc and 1 M HCl. After filtration to remove insoluble material, the organic layer was separated, dried over MgSO₄, filtered, and concentrated. The resulting solids were suspended in MeOH and then filtered to give 2-((3-isopropyl-5-(trifluoromethoxy)benzyl)amino)pyrimidine-5-carboxylic acid (1.0 g, 28% yield).

### Step 2: (2-((3-Isopropyl-5-(trifluoromethoxy)benzyl)amino)pyrimidin-5-yl)(6-oxa-1-azaspiro[3.3]heptan-1-yl)methanone

To a solution of 2-((3-isopropyl-5-(trifluoromethoxy)benzyl)amino)pyrimidine-5-carboxylic acid (0.12 g, 0.34 mmol, 1.0 eq.) in DMF (2.0 mL) was added 6-oxa-1-azaspiro[3.3]heptane hemioxalate (intermediate B1, 0.059 g, 0.41 mmol, 1.2 eq.), HATU (0.26 g, 0.68 mmol, 2.0 eq.), and DIPEA (0.30 mL, 1.7 mmol, 5.0 eq.). After 0.5 h, the mixture was loaded directly onto a reverse phase C₁₈ column for purification to give (2-((3-isopropyl-5-(trifluoromethoxy)benzyl)amino)pyrimidin-5-yl)(6-oxa-1-azaspiro[3.3]heptan-1-yl)methanone (0.098 g, 67% yield). MS (ESI): m/z=437 [M+H]⁺

### Procedure B: SNAr with ethyl 2-chloropyrimidine-5-carboxylate followed by hydrolysis and amide coupling

### Step 1: Ethyl 2-((3-isopropyl-5-(trifluoromethoxy)benzyl)amino)pyrimidine-5-carboxylate

To a solution of (3-isopropyl-5-(trifluoromethoxy)phenyl)methanamine hydrochloride (intermediate A37, 4.0 g, 15 mmol, 1.0 eq.) in 2-methoxyethanol (32 mL) was added ethyl 2-chloropyrimidine-5-carboxylate (2.8 g, 15 mmol, 1.0 eq.) and DIPEA (7.8 mL, 45 mmol, 3.0 eq.). The mixture was heated to 120° C. for 20 h then cooled to RT, diluted with water and extracted three times with EtOAc. The combined organic extracts were washed with brine, dried over MgSO₄, filtered, and concentrated. The crude material was purified on SiO₂ to give ethyl 2-((3-isopropyl-5-(trifluoromethoxy)benzyl)amino)pyrimidine-5-carboxylate (5.0 g, 87% yield).

### Step 2: 2-((3-Isopropyl-5-(trifluoromethoxy)benzyl)amino)pyrimidine-5-carboxylic acid

To a solution of ethyl 2-((3-isopropyl-5-(trifluoromethoxy)benzyl)amino)pyrimidine-5-carboxylate (5.0 g, 13 mmol, 1.0 eq.) in THF (40 mL) and EtOH (30 mL) was added 3 M NaOH (15 mL, 46 mmol, 3.5 eq.). After 16 h, 3 M HCl (15 mL, 46 mmol, 3.5 eq.) was added and the resulting suspension was partially concentrated to remove the organics. Filtration provided 2-((3-isopropyl-5-(trifluoromethoxy)benzyl)amino)pyrimidine-5-carboxylic acid (4.5 g, 98% yield).

### Step 3: (2-((3-Isopropyl-5-(trifluoromethoxy)benzyl)amino)pyrimidin-5-yl)(6-oxa-1-azaspiro[3.3]heptan-1-yl)methanone

Prepared according to step 2 of Procedure A to give (2-((3-isopropyl-5-(trifluoromethoxy)benzyl)amino)pyrimidin-5-yl)(6-oxa-1-azaspiro[3.3]heptan-1-yl)methanone. MS (ESI): m/z=437 [M+H]⁺

The examples in the following table were synthesized in analogy to Example 1 using the building blocks and procedure specified:

| | | | Building Blocks | | |
|---|---|---|---|---|---|
| Ex. | Structure/Systemic Name | Proc. | A | B | MS, m/z |
| 2 | 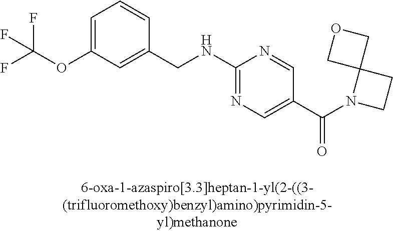 [SMILES: O=C(c1cnc(NCc2cccc(OC(F)(F)F)c2)nc1)N1CCC12COC2] | B | A1 | B1 | 395 [M + H]⁺ |
| 3 | 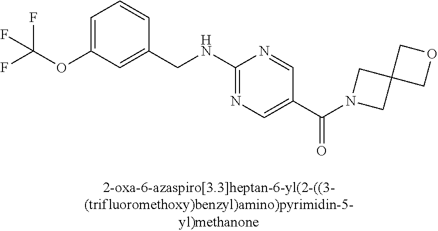 [SMILES: O=C(c1cnc(NCc2cccc(OC(F)(F)F)c2)nc1)N1CC2(COC2)C1] | B | A1 | B2 | 395 [M + H]⁺ |
| 4 | 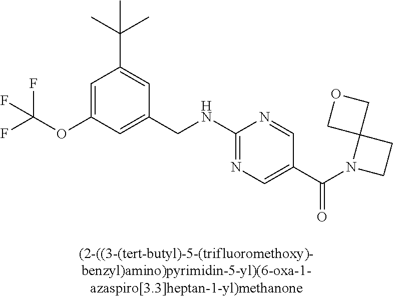 [SMILES: CC(C)(C)c1cc(CNc2ncc(C(=O)N3CCC34COC4)cn2)cc(OC(F)(F)F)c1] | B | A27 | B1 | 451 [M + H]⁺ |
| 5 | 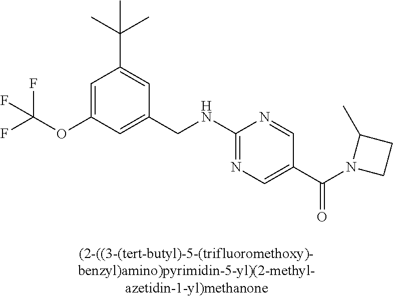 [SMILES: CC1CCN1C(=O)c1cnc(NCc2cc(OC(F)(F)F)cc(C(C)(C)C)c2)nc1] | B | A27 | B3 | 423 [M + H]⁺ |
| 6 | 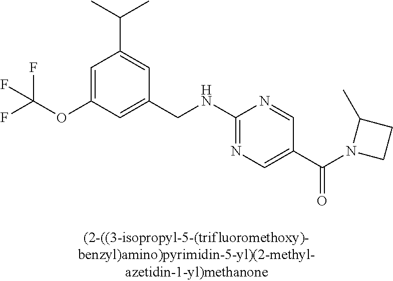 [SMILES: CC(C)c1cc(CNc2ncc(C(=O)N3CCC3C)cn2)cc(OC(F)(F)F)c1] | B | A37 | B3 | 409 [M + H]⁺ |
| 7 | 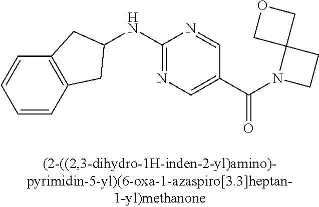 [SMILES: O=C(c1cnc(NC2Cc3ccccc3C2)nc1)N1CCC12COC2] | B | A2 | B1 | 337 [M + H]⁺ |
| 8 | 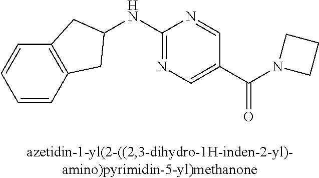 [SMILES: O=C(c1cnc(NC2Cc3ccccc3C2)nc1)N1CCC1] | B | A2 | B4 | 295 [M + H]⁺ |
| 9 | 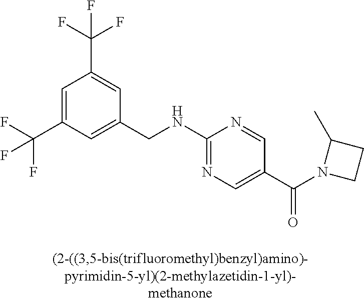 [SMILES: CC1CCN1C(=O)c1cnc(NCc2cc(C(F)(F)F)cc(C(F)(F)F)c2)nc1] | B | A3 | B3 | 419 [M + H]⁺ |
| 10 | 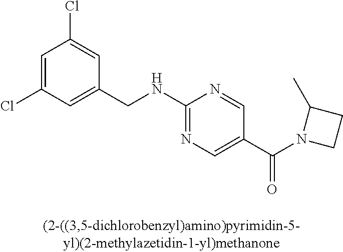 [SMILES: CC1CCN1C(=O)c1cnc(NCc2cc(Cl)cc(Cl)c2)nc1] | B | A4 | B3 | 351 [M + H]⁺ |
| 11 | 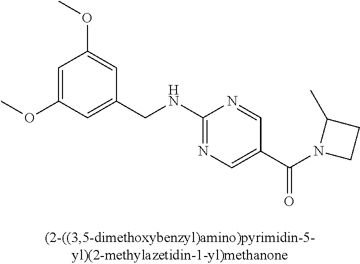 [SMILES: COc1cc(CNc2ncc(C(=O)N3CCC3C)cn2)cc(OC)c1] | B | A5 | B3 | 343 [M + H]⁺ |
| 12 | 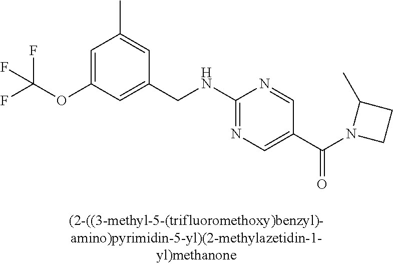 [SMILES: Cc1cc(CNc2ncc(C(=O)N3CCC3C)cn2)cc(OC(F)(F)F)c1] | B | A6 | B3 | 381 [M + H]⁺ |
| 13 | 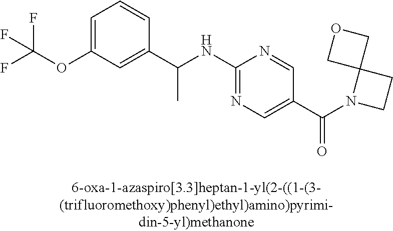 [SMILES: CC(Nc1ncc(C(=O)N2CCC23COC3)cn1)c1cccc(OC(F)(F)F)c1] | B | A7 | B1 | 409 [M + H]⁺ |
| 14 | 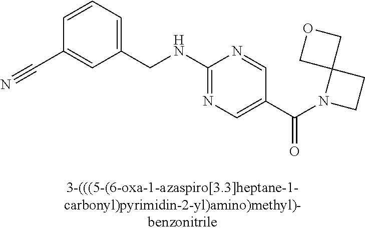 [SMILES: N#Cc1cccc(CNc2ncc(C(=O)N3CCC34COC4)cn2)c1] | A | A8 | B1 | 336 [M + H]⁺ |
| 15 | 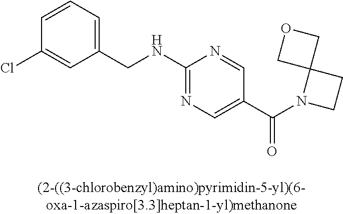 [SMILES: O=C(c1cnc(NCc2cccc(Cl)c2)nc1)N1CCC12COC2] | A | A9 | B1 | 345 [M + H]⁺ |
| 16 | 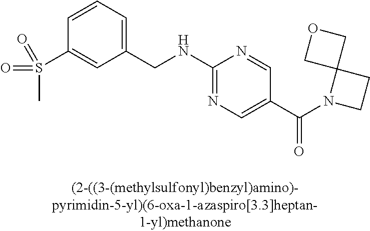 [SMILES: CS(=O)(=O)c1cccc(CNc2ncc(C(=O)N3CCC34COC4)cn2)c1] | A | A10 | B1 | 389 [M + H]⁺ |
| 17 | 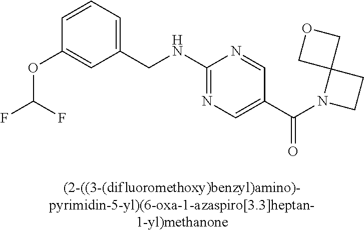 [SMILES: O=C(c1cnc(NCc2cccc(OC(F)F)c2)nc1)N1CCC12COC2] | A | A11 | B1 | 377 [M + H]⁺ |
| 18 | 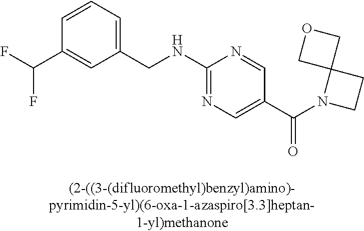 [SMILES: O=C(c1cnc(NCc2cccc(C(F)F)c2)nc1)N1CCC12COC2] | A | A12 | B1 | 361 [M + H]⁺ |
| 19 | 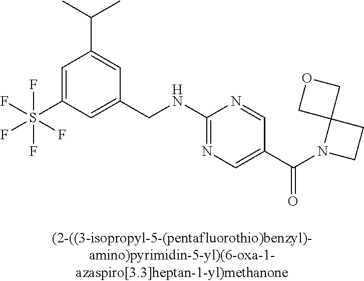 [SMILES: CC(C)c1cc(CNc2ncc(C(=O)N3CCC34COC4)cn2)cc(S(F)(F)(F)(F)F)c1] | A | A38 | B1 | 479 [M + H]⁺ |
| 20 | 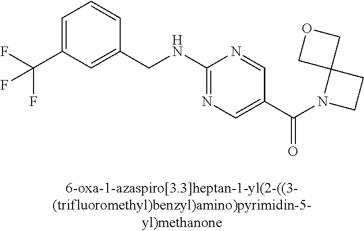 [SMILES: O=C(c1cnc(NCc2cccc(C(F)(F)F)c2)nc1)N1CCC12COC2] | A | A13 | B1 | 379 [M + H]⁺ |
| 21 | 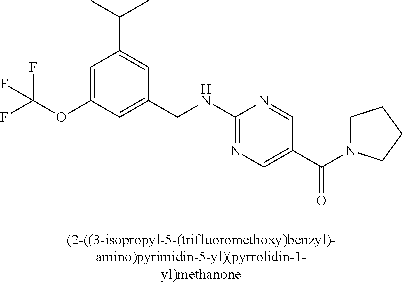 [SMILES: CC(C)c1cc(CNc2ncc(C(=O)N3CCCC3)cn2)cc(OC(F)(F)F)c1] | B | A37 | B11 | 409 [M + H]⁺ |
| 22 | 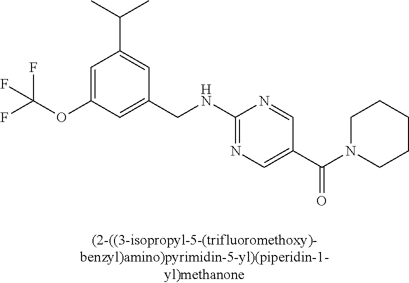 [SMILES: CC(C)c1cc(CNc2ncc(C(=O)N3CCCCC3)cn2)cc(OC(F)(F)F)c1] | B | A37 | B12 | 423 [M + H]⁺ |
| 23 | 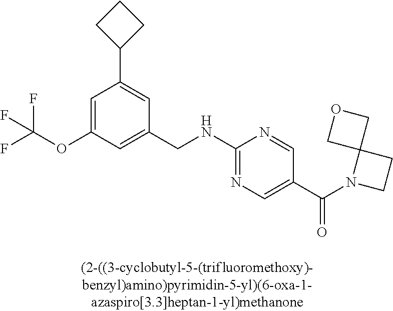 [SMILES: O=C(c1cnc(NCc2cc(OC(F)(F)F)cc(C3CCC3)c2)nc1)N1CCC12COC2] | A | A28 | B1 | 449 [M + H]⁺ |
| 24 | 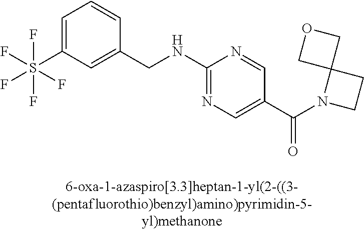 [SMILES: O=C(c1cnc(NCc2cccc(S(F)(F)(F)(F)F)c2)nc1)N1CCC12COC2] | A | A14 | B1 | 437 [M + H]⁺ |
| 25 | 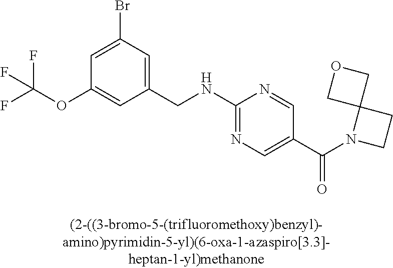 [SMILES: O=C(c1cnc(NCc2cc(Br)cc(OC(F)(F)F)c2)nc1)N1CCC12COC2] | B | A15 | B1 | 473 [M + H]⁺ |
| 26 | 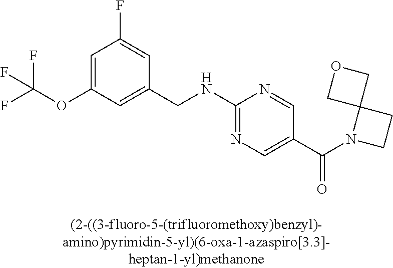 [SMILES: O=C(c1cnc(NCc2cc(F)cc(OC(F)(F)F)c2)nc1)N1CCC12COC2] | A | A16 | B1 | 413 [M + H]⁺ |
| 27 | 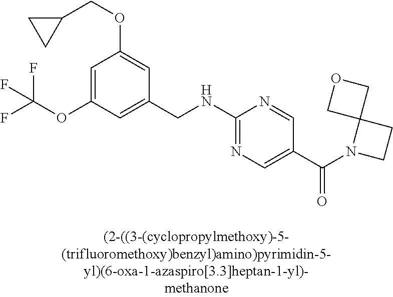 [SMILES: O=C(c1cnc(NCc2cc(OCC3CC3)cc(OC(F)(F)F)c2)nc1)N1CCC12COC2] | A | A30 | B1 | 465 [M + H]⁺ |
| 28 | 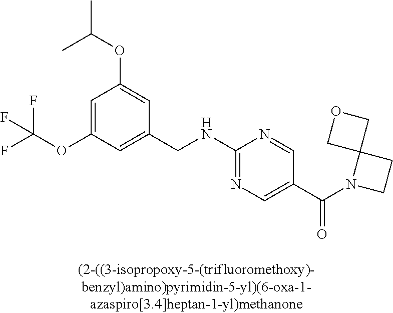 [SMILES: CC(C)Oc1cc(CNc2ncc(C(=O)N3CCC34COC4)cn2)cc(OC(F)(F)F)c1] | A | A29 | B1 | 453 [M + H]⁺ |
| 29 | 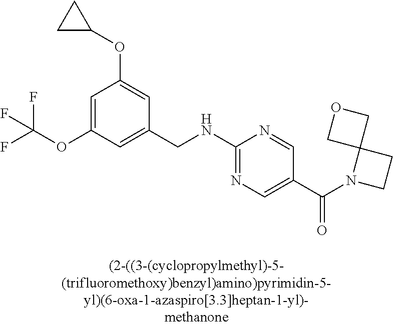 [SMILES: O=C(c1cnc(NCc2cc(OC3CC3)cc(OC(F)(F)F)c2)nc1)N1CCC12COC2] | A | A36 | B1 | 449 [M + H]⁺ |
| 30 | 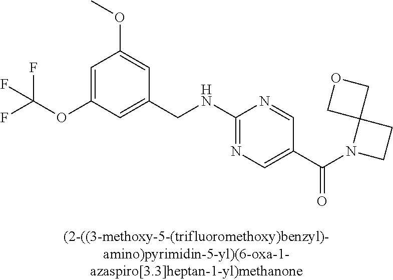 [SMILES: COc1cc(CNc2ncc(C(=O)N3CCC34COC4)cn2)cc(OC(F)(F)F)c1] | A | A31 | B1 | 425 [M + H]⁺ |
| 31 | 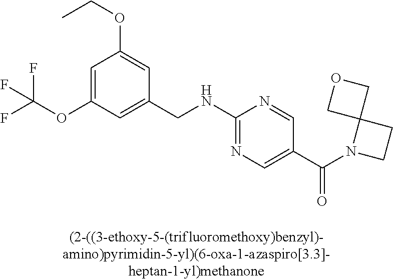 [SMILES: CCOc1cc(CNc2ncc(C(=O)N3CCC34COC4)cn2)cc(OC(F)(F)F)c1] | A | A32 | B1 | 439 [M + H]⁺ |
| 32 | 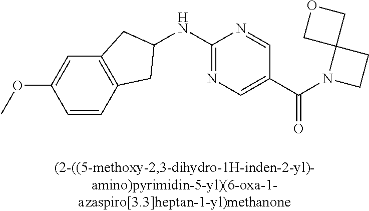 [SMILES: COc1ccc2c(c1)CC(Nc1ncc(C(=O)N3CCC34COC4)cn1)C2] | B | A17 | B1 | 367 [M + H]⁺ |
| 33 | 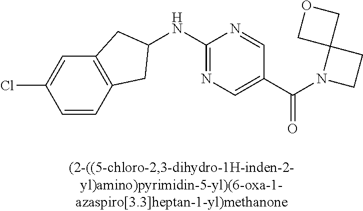 [SMILES: O=C(c1cnc(NC2Cc3ccc(Cl)cc3C2)nc1)N1CCC12COC2] | B | A18 | B1 | 371 [M + H]⁺ |
| 34 | 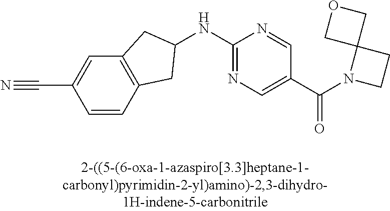 [SMILES: N#Cc1ccc2c(c1)CC(Nc1ncc(C(=O)N3CCC34COC4)cn1)C2] | B | A19 | B1 | 362 [M + H]⁺ |
| 35 | 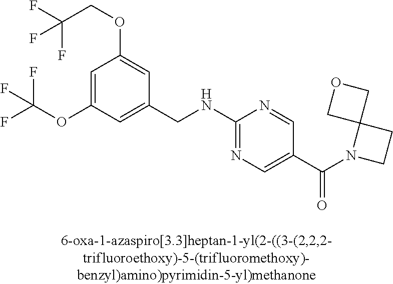 [SMILES: O=C(c1cnc(NCc2cc(OCC(F)(F)F)cc(OC(F)(F)F)c2)nc1)N1CCC12COC2] | B | A33 | B1 | 493 [M + H]⁺ |
| 36 | 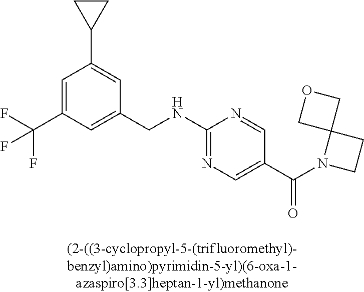 [SMILES: O=C(c1cnc(NCc2cc(C3CC3)cc(C(F)(F)F)c2)nc1)N1CCC12COC2] | A | A40 | B1 | 419 [M + H]⁺ |
| 37 | 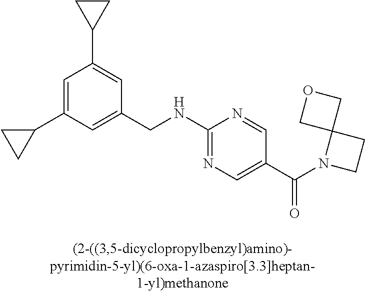 [SMILES: O=C(c1cnc(NCc2cc(C3CC3)cc(C3CC3)c2)nc1)N1CCC12COC2] | B | A41 | B1 | 391 [M + H]⁺ |
| 38 | 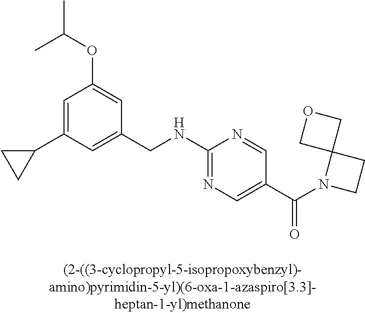 [SMILES: CC(C)Oc1cc(CNc2ncc(C(=O)N3CCC34COC4)cn2)cc(C2CC2)c1] | B | A42 | B1 | 409 [M + H]⁺ |
| 39 | 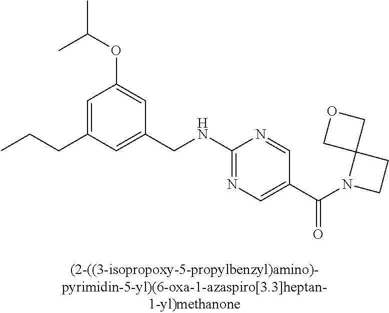 [SMILES: CCCc1cc(CNc2ncc(C(=O)N3CCC34COC4)cn2)cc(OC(C)C)c1] | B | A43 | B1 | 411 [M + H]⁺ |
| 40 | 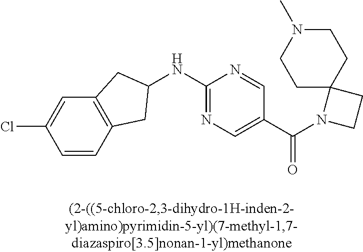 [SMILES: CN1CCC2(CC1)CCN2C(=O)c1cnc(NC2Cc3ccc(Cl)cc3C2)nc1] | B | A18 | B37 | 412 [M + H]⁺ |
| 41 | 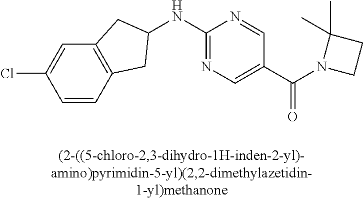 [SMILES: CC1(C)CCN1C(=O)c1cnc(NC2Cc3ccc(Cl)cc3C2)nc1] | B | A18 | B13 | 357 [M + H]⁺ |
| 42 | 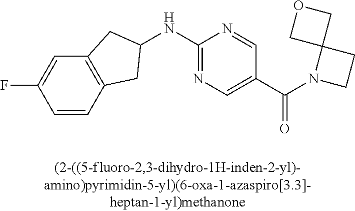 [SMILES: O=C(c1cnc(NC2Cc3ccc(F)cc3C2)nc1)N1CCC12COC2] | B | A20 | B1 | 355 [M + H]⁺ |
| 43 | 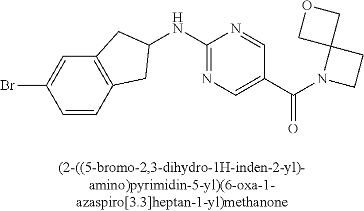 [SMILES: O=C(c1cnc(NC2Cc3ccc(Br)cc3C2)nc1)N1CCC12COC2] | B | A21 | B1 | 415 [M + H]⁺ |
| 44 | 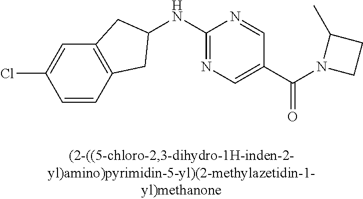 [SMILES: CC1CCN1C(=O)c1cnc(NC2Cc3ccc(Cl)cc3C2)nc1] | B | A18 | B3 | 343 [M + H]⁺ |
| 45 | 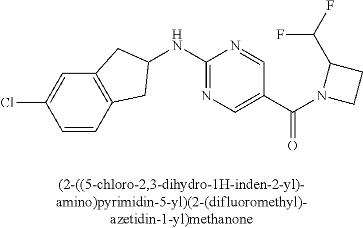 [SMILES: O=C(c1cnc(NC2Cc3ccc(Cl)cc3C2)nc1)N1CCC1C(F)F] | B | A18 | B38 | 379 [M + H]⁺ |
| 46 | 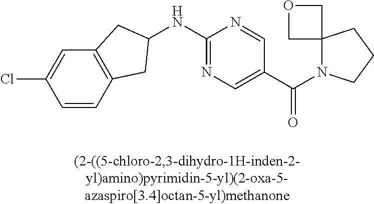 [SMILES: O=C(c1cnc(NC2Cc3ccc(Cl)cc3C2)nc1)N1CCCC12COC2] | B | A18 | B21 | 385 [M + H]⁺ |
| 47 | 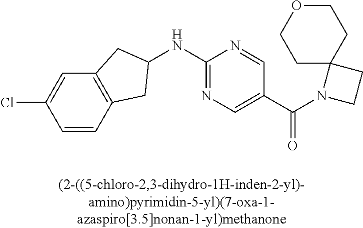 [SMILES: O=C(c1cnc(NC2Cc3ccc(Cl)cc3C2)nc1)N1CCC12CCOCC2] | B | A18 | B26 | 399 [M + H]⁺ |
| 48 | 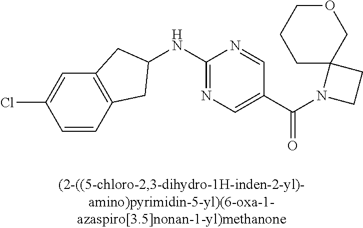 [SMILES: O=C(c1cnc(NC2Cc3ccc(Cl)cc3C2)nc1)N1CCC12CCCOC2] | B | A18 | B33 | 399 [M + H]⁺ |
| 49 | 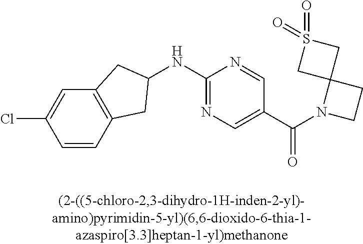 [SMILES: O=C(c1cnc(NC2Cc3ccc(Cl)cc3C2)nc1)N1CCC12CS(=O)(=O)C2] | B | A18 | B32 | 419 [M + H]⁺ |
| 50 | 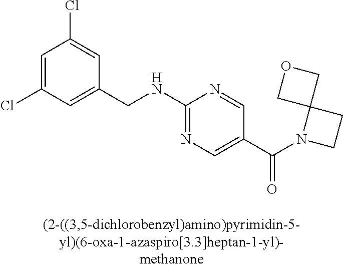 [SMILES: O=C(c1cnc(NCc2cc(Cl)cc(Cl)c2)nc1)N1CCC12COC2] | B | A4 | B1 | 379 [M + H]⁺ |
| 51 | 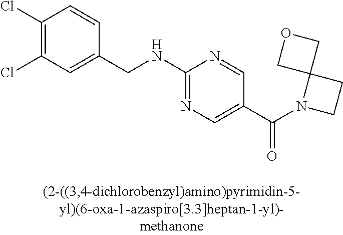 [SMILES: O=C(c1cnc(NCc2ccc(Cl)c(Cl)c2)nc1)N1CCC12COC2] | B | A22 | B1 | 379 [M + H]⁺ |
| 52 | 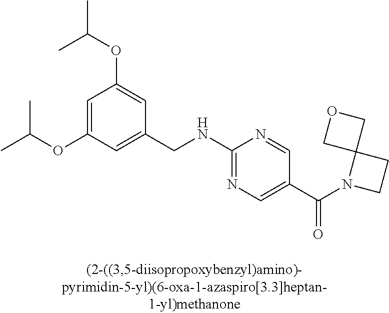 [SMILES: CC(C)Oc1cc(CNc2ncc(C(=O)N3CCC34COC4)cn2)cc(OC(C)C)c1] | B | A34 | B1 | 427 [M + H]⁺ |
| 53 | 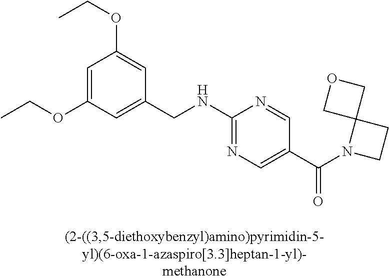 [SMILES: CCOc1cc(CNc2ncc(C(=O)N3CCC34COC4)cn2)cc(OCC)c1] | B | A35 | B1 | 399 [M + H]⁺ |
| 54 | 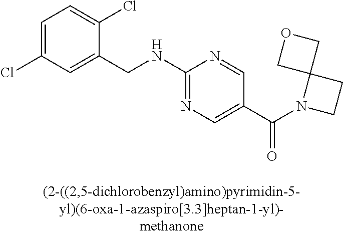 [SMILES: O=C(c1cnc(NCc2cc(Cl)ccc2Cl)nc1)N1CCC12COC2] | B | A23 | B1 | 379 [M + H]⁺ |
| 55 | 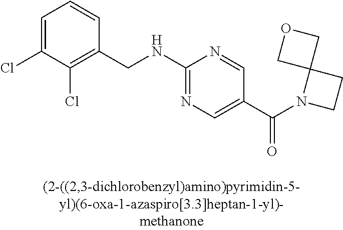 [SMILES: O=C(c1cnc(NCc2cccc(Cl)c2Cl)nc1)N1CCC12COC2] | B | A24 | B1 | 379 [M + H]⁺ |
| 56 | 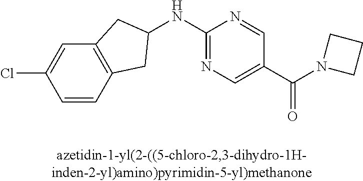 [SMILES: O=C(c1cnc(NC2Cc3ccc(Cl)cc3C2)nc1)N1CCC1] | B | A18 | B4 | 329 [M + H]⁺ |
| 57 | 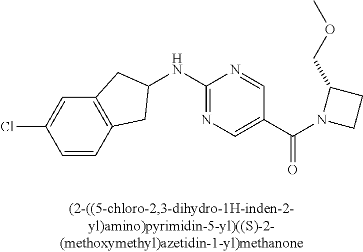 [SMILES: COC[C@@H]1CCN1C(=O)c1cnc(NC2Cc3ccc(Cl)cc3C2)nc1] | B | A18 | B29 | 373 [M + H]⁺ |
| 58 | 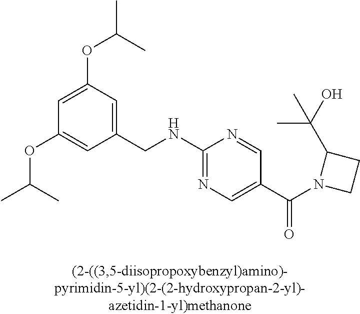 [SMILES: CC(C)Oc1cc(CNc2ncc(C(=O)N3CCC3C(C)(C)O)cn2)cc(OC(C)C)c1] | B | A34 | B43 | 443 [M + H]⁺ |
| 59 | 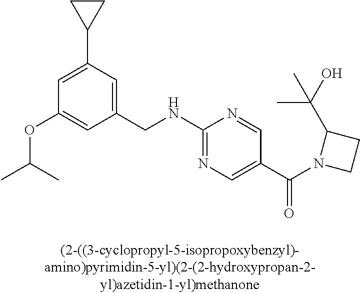 [SMILES: CC(C)Oc1cc(CNc2ncc(C(=O)N3CCC3C(C)(C)O)cn2)cc(C2CC2)c1] | B | A42 | B43 | 425 [M + H]⁺ |
| 60 | 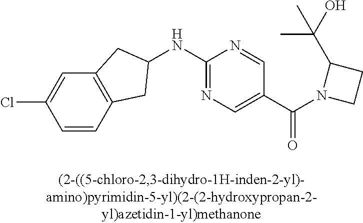 [SMILES: CC(C)(O)C1CCN1C(=O)c1cnc(NC2Cc3ccc(Cl)cc3C2)nc1] | B | A18 | B43 | 387 [M + H]⁺ |
| 61 | 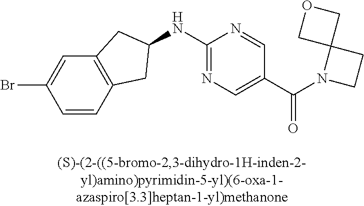 [SMILES: O=C(c1cnc(N[C@H]2Cc3ccc(Br)cc3C2)nc1)N1CCC12COC2] | B | A25 | B1 | 415 [M + H]⁺ |
| 62 | 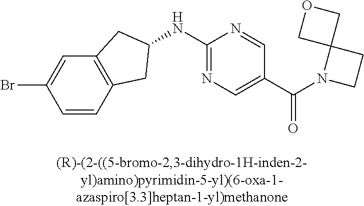 [SMILES: O=C(c1cnc(N[C@@H]2Cc3ccc(Br)cc3C2)nc1)N1CCC12COC2] | B | A26 | B1 | 415 [M + H]⁺ |
| 63 | 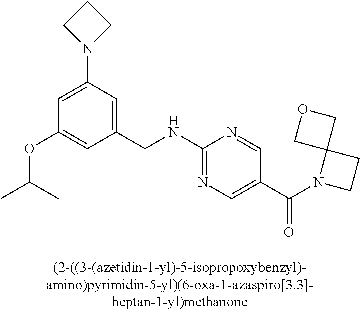 [SMILES: CC(C)Oc1cc(CNc2ncc(C(=O)N3CCC34COC4)cn2)cc(N2CCC2)c1] | B | A46 | B1 | 424 [M + H]⁺ |
| 64 | 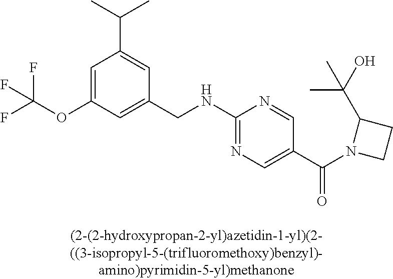 [SMILES: CC(C)c1cc(CNc2ncc(C(=O)N3CCC3C(C)(C)O)cn2)cc(OC(F)(F)F)c1] | B | A37 | B43 | 453 [M + H]⁺ |
| 65 | 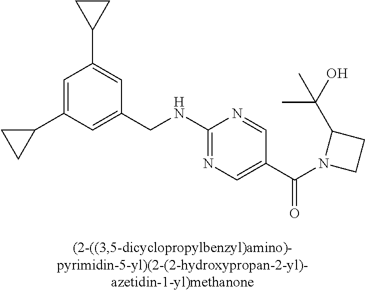 [SMILES: CC(C)(O)C1CCN1C(=O)c1cnc(NCc2cc(C3CC3)cc(C3CC3)c2)nc1] | B | A41 | B43 | 407 [M + H]⁺ |
| 66 | 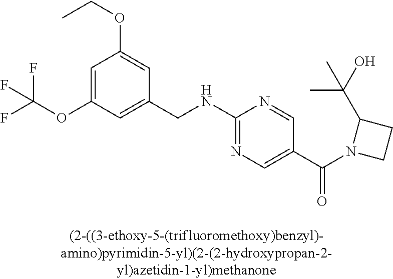 [SMILES: CCOc1cc(CNc2ncc(C(=O)N3CCC3C(C)(C)O)cn2)cc(OC(F)(F)F)c1] | A | A32 | B43 | 455 [M + H]⁺ |
| 67 | 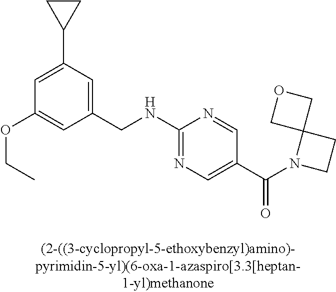 [SMILES: CCOc1cc(CNc2ncc(C(=O)N3CCC34COC4)cn2)cc(C2CC2)c1] | B | A44 | B1 | 395 [M + H]⁺ |
| 68 | 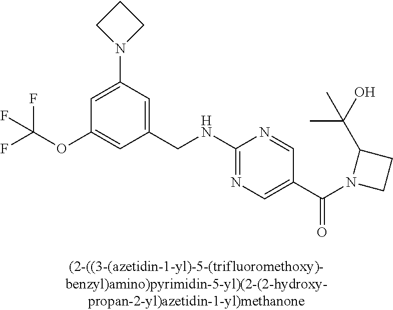 [SMILES: CC(C)(O)C1CCN1C(=O)c1cnc(NCc2cc(OC(F)(F)F)cc(N3CCC3)c2)nc1] | B | A47 | B43 | 466 [M + H]⁺ |

### Example 69 and Example 70. (2-((3-Isopropyl-5-(trifluoromethoxy)benzyl)amino) pyrimidin-5-yl)(2-methylazetidin-1-yl)methanone, S and R Enantiomers

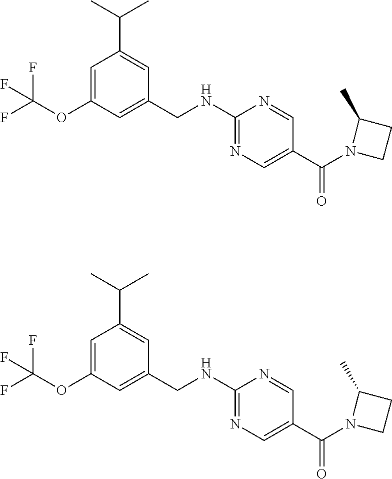 [SMILES: CC(C)c1cc(CNc2ncc(C(=O)N3CC[C@@H]3C)cn2)cc(OC(F)(F)F)c1 ; CC(C)c1cc(CNc2ncc(C(=O)N3CC[C@H]3C)cn2)cc(OC(F)(F)F)c1]

Racemic product of example 6 was resolved by chiral SFC (stationary phase: Lux Cellulose-2 10×250 mm, 5 μm; mobile phase: 5000 methanol, 100 Bar of CO₂; flow rate: 10 mL/min) into its two enantio-enriched (>99% ee) antipodes. First eluting enantiomer, example 69; RT: 3.56 min, MS (ESI): m/z=409 [M+H]⁺. Second eluting enantiomer, example 70; RT: 5.58 mun, MS (ESI): m/z=409 [M+H]⁺.

### Example 71.6-Oxa-1-azaspiro[3.3]heptan-1-yl(5-((3-(trifluoromethoxy)benzyl)|amino)pyrazin-2-yl)methanone

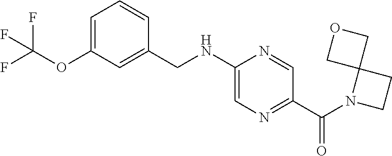 [SMILES: O=C(c1cnc(NCc2cccc(OC(F)(F)F)c2)cn1)N1CCC12COC2]

The title compound was prepared according to Procedure B in Example 1 using ethyl 5-chloropyrazine-2-carboxylate and (3-(trifluoromethoxy)phenyl)methanamine (intermediate A1) in step 1. MS (ESI): m/z=395 [M+H]⁺

### Example 72. 6-Oxa-1-azaspiro[3.3]heptan-1-yl(6-((3-(trifluoromethoxy) benzyl)amino)pyridazin-3-yl)methanone

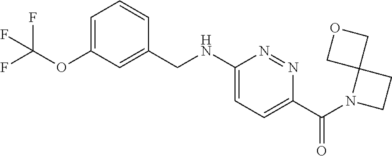 [SMILES: O=C(c1ccc(NCc2cccc(OC(F)(F)F)c2)nn1)N1CCC12COC2]

The title compound was prepared according to Procedure B in Example 1 using ethyl 6-chloropyridazine-3-carboxylate and (3-(trifluoromethoxy)phenyl)methanamine (intermediate A1) in step 1. MS (ESI): m/z=395 [M+H]⁺

### Example 73. 6-Oxa-1-azaspiro[3.3]heptan-1-yl(6-((3-(trifluoromethoxy) benzyl)amino)pyridin-3-yl)methanone

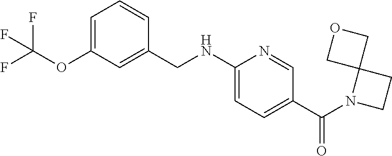 [SMILES: O=C(c1ccc(NCc2cccc(OC(F)(F)F)c2)nc1)N1CCC12COC2]

The title compound was prepared according to Procedure B in Example 1 using ethyl 6-chloronicotinate and (3-(trifluoromethoxy)phenyl)methanamine (intermediate A1) in step 1. MS (ESI): m/z=394 [M+H]⁺

### Example 74. Azetidin-1-yl(5-((3-isopropyl-5-(trifluoromethoxy) benzyl)amino)pyrazin-2-yl)methanone

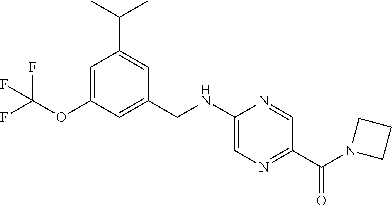 [SMILES: CC(C)c1cc(CNc2cnc(C(=O)N3CCC3)cn2)cc(OC(F)(F)F)c1]

The title compound was prepared according to Procedure B in Example 1 using ethyl 5-chloropyrazine-2-carboxylate in step 1 and azetidine hydrochloride (intermediate B4) in step 3. MS (ESI): m/z=395 [M+H]⁺

### Example 75. (5-((3-Isopropyl-5-(trifluoromethoxy)benzyl)amino) pyrazin-2-yl)(6-oxa-1-azaspiro[3.3]heptan-1-yl)methanone

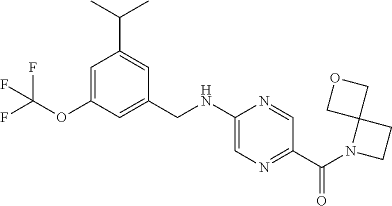 [SMILES: CC(C)c1cc(CNc2cnc(C(=O)N3CCC34COC4)cn2)cc(OC(F)(F)F)c1]

The title compound was prepared according to Procedure B in Example 1 using ethyl 5-chloropyrazine-2-carboxylate in step 1. MS (ESI): m/z=437 [M+H]⁺

### Example 76. (6-((3-Isopropyl-5-(trifluoromethoxy)benzyl)amino) pyridin-3-yl)(6-oxa-1-azaspiro[3.3]heptan-1-yl)methanone

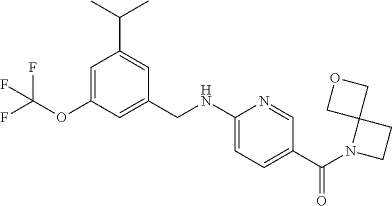 [SMILES: CC(C)c1cc(CNc2ccc(C(=O)N3CCC34COC4)cn2)cc(OC(F)(F)F)c1]

The title compound was prepared according to Procedure B in Example 1 using ethyl 6-chloronicotinate in step 1. MS (ESI): m/z=436 [M+H]⁺

### Example 77. (6-((3-Cyclopropyl-5-(trifluoromethoxy)benzyl)amino) pyridin-3-yl)(6-oxa-1-azaspiro[3.3]heptan-1-yl)methanone

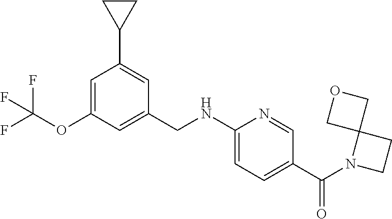 [SMILES: O=C(c1ccc(NCc2cc(OC(F)(F)F)cc(C3CC3)c2)nc1)N1CCC12COC2]

The title compound was prepared according to Procedure B in Example 1 using ethyl 6-chloronicotinate and (3-cyclopropyl-5-(trifluoromethoxy)phenyl)methanamine (intermediate A39) in step 1. MS (ESI): m/z=434 [M+H]⁺

### Example 78. (6-((5-Chloro-2,3-dihydro-1H-inden-2-yl)amino) pyridin-3-yl)(6-oxa-1-azaspiro[3.3]heptan-1-yl)methanone

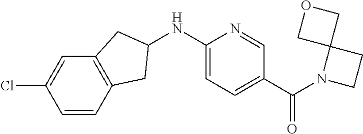 [SMILES: O=C(c1ccc(NC2Cc3ccc(Cl)cc3C2)nc1)N1CCC12COC2]

The title compound was prepared according to Procedure B in Example 1 using ethyl 6-chloronicotinate and 5-chloro-2,3-dihydro-1H-inden-2-amine (intermediate A18) in step 1. MS (ESI): m/z=370 [M+H]⁺

### Example 79. (2-((3-Cyclopropyl-5-(trifluoromethoxy)benzyl)amino) pyrimidin-5-yl)(1-azaspiro[3.3]heptan-1-yl)methanone

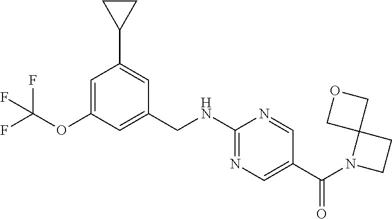 [SMILES: O=C(c1cnc(NCc2cc(OC(F)(F)F)cc(C3CC3)c2)nc1)N1CCC12COC2]

### Step 1: Ethyl 2-((3-bromo-5-(trifluoromethoxy)benzyl)amino)pyrimidine-5-carboxylate

To (3-bromo-5-(trifluoromethoxy)phenyl)methanamine (intermediate A15, 3.0 g, 11 mmol, 1.0 eq.) in 2-methoxyethanol (24 mL) was added ethyl 2-chloropyrimidine-5-carboxylate (2.1 g, 11 mmol, 1.0 eq.) and DIPEA (5.8 mL, 33 mmol, 3.0 eq.). The resulting solution was heated to 120° C. for 22 h and then cooled to RT and partitioned between EtOAc and water. The organic layer was separated, washed with brine, dried over MgSO₄, filtered and concentrated. The resulting solid was triturated with hexanes and filtered to give ethyl 2-((3-bromo-5-(trifluoromethoxy)benzyl)amino)pyrimidine-5-carboxylate (4.0 g, 86% yield).

### Step 2: Ethyl 2-((3-cyclopropyl-5-(trifluoromethoxy)benzyl)amino)pyrimidine-5-carboxylate

To ethyl 2-((3-bromo-5-(trifluoromethoxy)benzyl)amino)pyrimidine-5-carboxylate (2.0 g, 4.8 mmol, 1.0 eq.) in 1,4-dioxane (20 mL) was added cyclopropylboronic acid (1.6 g, 19 mmol, 4.0 eq.) and cesium fluoride (2.5 g, 16 mmol, 3.4 eq.). N₂ was bubbled through the mixture for 10 minutes. PdCl₂(dppf).CH₂Cl₂ (0.35 g, 0.48 mmol, 10 mol %) was added and the reaction vessel was sealed and heated to 90° C. for 4 h. The mixture was partitioned between EtOAc and water. The organic layer was separated, washed with brine, dried over MgSO₄, filtered and concentrated. The residue was purified on SiO₂ to give ethyl 2-((3-cyclopropyl-5-(trifluoromethoxy)benzyl)amino)pyrimidine-5-carboxylate (1.7 g, 94% yield).

### Step 3: 2-((3-Cyclopropyl-5-(trifluoromethoxy)benzyl)amino)pyrimidine-5-carboxylic acid

To a solution of ethyl 2-((3-cyclopropyl-5-(trifluoromethoxy)benzyl)amino)pyrimidine-5-carboxylate (1.7 g, 4.5 mmol) in THF (12 mL) and EtOH (8 mL) was added 3 M NaOH (5.2 mL, 16 mmol, 3.5 eq.). The mixture was stirred at RT for 20 h, then neutralized with 3 M HCl (5.2 mL, 16 mmol, 3.5 eq.). The mixture was concentrated to remove the organics then filtered and dried to give 2-((3-cyclopropyl-5-(trifluoromethoxy)-benzyl)amino)pyrimidine-5-carboxylic acid (1.4 g, 9000 yield).

### Step 4: (2-((3-Cyclopropyl-5-(trifluoromethoxy)benzyl)amino)pyrimidin-5-yl)(1-azaspiro[3.3]-heptan-1-yl)methanone

To a solution of 2-((3-cyclopropyl-5-(trifluoromethoxy)benzyl)amino)pyrimidine-5-carboxylic acid (0.050 g, 0.14 mmol, 1.0 eq.) in DMF (1 mL) was added 1-azaspiro[3.3]heptane (intermediate B14, 0.017 g, 0.17 mmol, 1.2 eq.), HATU (0.059 g, 0.15 mmol, 1.1 eq.), and DIPEA (0.075 mL, 0.42 mmol, 3.0 eq.). After 0.5 h, the mixture was loaded directly onto a reverse phase C₁₈ column for purification to give (2-((3-cyclopropyl-5-(trifluoromethoxy)benzyl)amino)pyrimidin-5-yl)(1-azaspiro[3.3]heptan-1-yl)methanone (0.056 g, 91% yield). MS (ESI): m/z=433 [M+H]⁺

The examples in the following table were synthesized in analogy to Example 79 using the specified building block in step 4:

| | | | Building | |
|---|---|---|---|---|
| Ex. | Structure | Systemic Name | Block | MS, m/z |
| 80 | 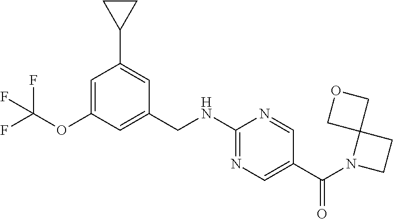 [SMILES: O=C(c1cnc(NCc2cc(OC(F)(F)F)cc(C3CC3)c2)nc1)N1CCC12COC2] | (2-((3-cyclopropyl-5- (trifluoromethoxy)benzyl)ami- no)pyrimidin-5-yl)(6-oxa-1- azaspiro[3.3]heptan-1-yl)- methanone | B1 | 435 [M + H]⁺ |
| 81 | 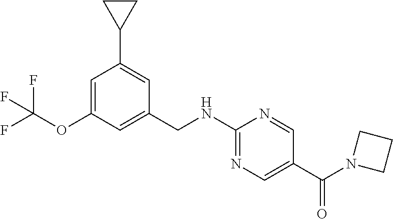 [SMILES: O=C(c1cnc(NCc2cc(OC(F)(F)F)cc(C3CC3)c2)nc1)N1CCC1] | azetidin-1-yl(2-((3- cyclopropyl-5-(trifluoro- methoxy)benzyl)amino)- pyrimidin-5-yl)methanone | B4 | 393 [M + H]⁺ |
| 82 | 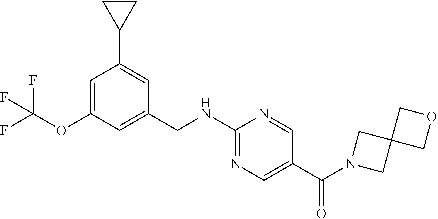 [SMILES: O=C(c1cnc(NCc2cc(OC(F)(F)F)cc(C3CC3)c2)nc1)N1CC2(COC2)C1] | (2-((3-cyclopropyl-5- (trifluoromethoxy)benzyl)ami- no)pyrimidin-5-yl)(2-oxa-6- azaspiro[3.3]heptan-6- yl)methanone | B2 | 435 [M + H]⁺ |
| 83 | 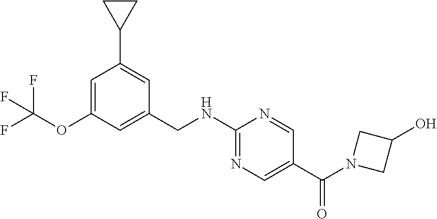 [SMILES: O=C(c1cnc(NCc2cc(OC(F)(F)F)cc(C3CC3)c2)nc1)N1CC(O)C1] | (2-((3-cyclopropyl-5- (trifluoromethoxy)benzyl)ami- no)pyrimidin-5-yl)(3- hydroxyazetidin-1-yl)- methanone | B5 | 409 [M + H]⁺ |
| 84 | 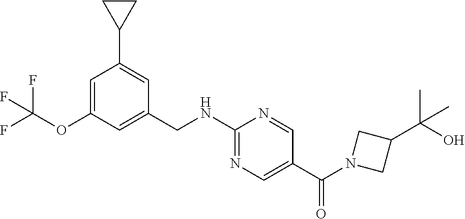 [SMILES: CC(C)(O)C1CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3-(2- hydroxypropan-2-yl)azetidin- 1-yl)methanone | B6 | 451 [M + H]⁺ |
| 85 | 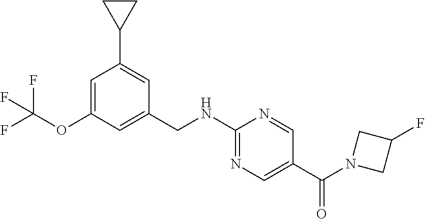 [SMILES: O=C(c1cnc(NCc2cc(OC(F)(F)F)cc(C3CC3)c2)nc1)N1CC(F)C1] | (2-((3-cyclopropyl-5- (trifluoromethoxy)benzyl)ami- no)pyrimidin-5-yl)(3- fluoroazetidin-1-yl)- methanone | B7 | 411 [M + H]⁺ |
| 86 | 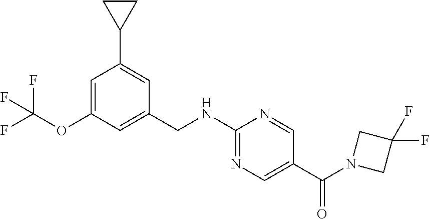 [SMILES: O=C(c1cnc(NCc2cc(OC(F)(F)F)cc(C3CC3)c2)nc1)N1CC(F)(F)C1] | (2-((3-cyclopropyl-5- (trifluoromethoxy)benzyl)ami- no)pyrimidin-5-yl)(3,3- difluroazetidin-1-yl)- methanone | B8 | 429 [M + H]⁺ |
| 87 | 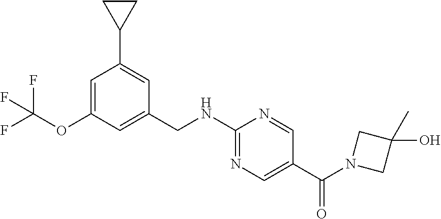 [SMILES: CC1(O)CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3- hydroxy-3-methylazetidin-1- yl)methanone | B9 | 423 [M + H]⁺ |
| 88 | 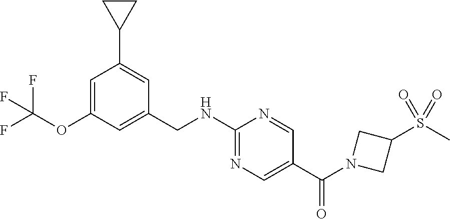 [SMILES: CS(=O)(=O)C1CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3- (methylsulfonyl)azetidin-1- yl)methanone | B10 | 471 [M + H]⁺ |
| 89 | 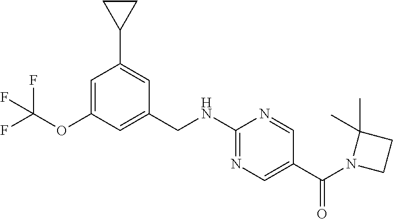 [SMILES: CC1(C)CCN1C(=O)c1cnc(NCc2cc(OC(F)(F)F)cc(C3CC3)c2)nc1] | (2-((3-cyclopropyl-5- (trifluoromethoxy)benzyl)ami- no)pyrimidin-5-yl)(2,2- dimethylazetidin-1- yl)methanone | B13 | 421 [M + H]⁺ |
| 90 | 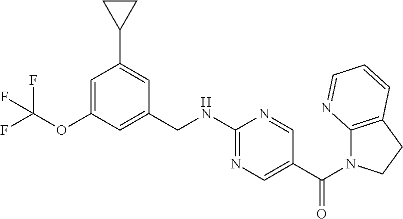 [SMILES: O=C(c1cnc(NCc2cc(OC(F)(F)F)cc(C3CC3)c2)nc1)N1CCc2cccnc21] | (2-((3-cyclopropyl-5- (trifluoromethoxy)benzyl)ami- no)pyrimidin-5-yl)(2,3- dihydro-1H-pyrrolo[2,3- b]pyridin-1-yl)methanone | B15 | 456 [M + H⁺ |
| 91 | 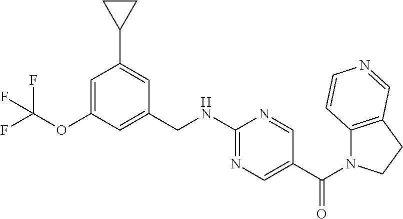 [SMILES: O=C(c1cnc(NCc2cc(OC(F)(F)F)cc(C3CC3)c2)nc1)N1CCc2cnccc21] | (2-((3-cyclopropyl-5- (trifluoromethoxy)benzyl)ami- no)pyrimidin-5-yl)(2,3- dihydro-1H-pyrrolo[3,2- c]pyridin-1-yl)methanone | B16 | 456 [M + H]⁺ |
| 92 | 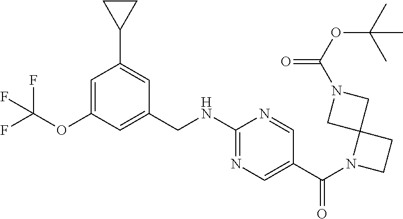 [SMILES: CC(C)(C)OC(=O)N1CC2(CCN2C(=O)c2cnc(NCc3cc(OC(F)(F)F)cc(C4CC4)c3)nc2)C1] | tert-butyl 1-(2-((3- cyclopropyl-5-(trifluoro- methoxy)benzyl)amino)py- rimidine-5-carbonyl)-1,6- diazaspiro[3.3]heptane-6- carboxylate | B17 | 534 [M + H]⁺ |
| 93 | 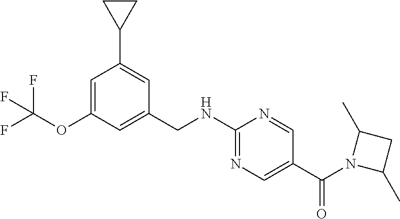 [SMILES: CC1CC(C)N1C(=O)c1cnc(NCc2cc(OC(F)(F)F)cc(C3CC3)c2)nc1] | (2-((3-cyclopropyl-5- (trifluoromethoxy)benzyl)ami- no)pyrimidin-5-yl)(2,4- dimethylazetidin-1- yl)methanone | B18 | 421 [M + H]⁺ |
| 94 | 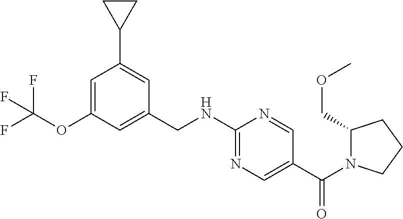 [SMILES: COC[C@@H]1CCCN1C(=O)c1cnc(NCc2cc(OC(F)(F)F)cc(C3CC3)c2)nc1] | (S)-(2-((3-cyclopropyl-5- (trifluoromethoxy)benzyl)ami- no)pyrimidin-5-yl)(2- (methoxymethyl)pyrrolidin- 1-yl)methanone | B19 | 451 [M + H]⁺ |
| 95 | 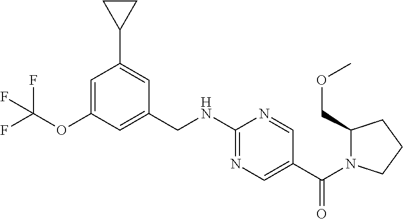 [SMILES: COC[C@H]1CCCN1C(=O)c1cnc(NCc2cc(OC(F)(F)F)cc(C3CC3)c2)nc1] | (R)-(2-((3-cyclopropyl-5- (trifluoromethxoy)benzyl)ami- no)pyrimidin-5-yl)(2- (methoxymethyl)pyrrolidin- 1-yl)methanone | B20 | 451 [M + H]⁺ |
| 96 | 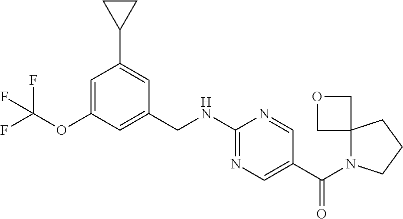 [SMILES: O=C(c1cnc(NCc2cc(OC(F)(F)F)cc(C3CC3)c2)nc1)N1CCCC12COC2] | (2-((3-cyclopropyl-5- (trifluoromethoxy)benzyl)ami- no)pyrimidin-5-yl)(2-oxa-5- azaspiro[3.4]octan-5- yl)methanone | B21 | 449 [M + H]⁺ |
| 97 | 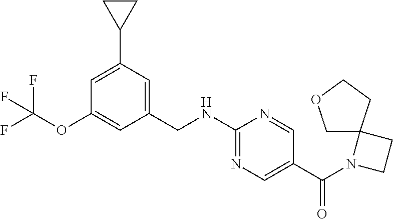 [SMILES: O=C(c1cnc(NCc2cc(OC(F)(F)F)cc(C3CC3)c2)nc1)N1CCC12CCOC2] | (2-((3-cyclopropyl-5- (trifluoromethoxy)benzyl)ami- no)pyrimidin-5-yl)(6-oxa-1- azaspiro[3.4]octan-1- yl)methanone | B22 | 449 [M + H]⁺ |
| 98 | 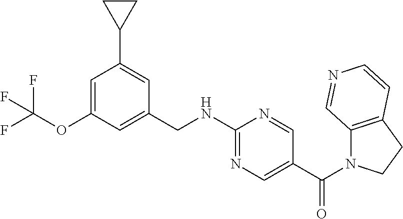 [SMILES: O=C(c1cnc(NCc2cc(OC(F)(F)F)cc(C3CC3)c2)nc1)N1CCc2ccncc21] | (2-((3-cyclopropyl-5- (trifluoromethoxy)benzyl)ami- no)pyrimidin-5-yl)(2,3- dihydro-1H-pyrrolo[2,3- c]pyridin-1-yl)methanone | B23 | 456 [M + H]⁺ |
| 99 | 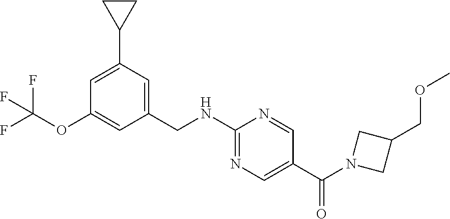 [SMILES: COCC1CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3- (methoxymethyl)azetidin-1- yl)methanone | B24 | 437 [M + H]⁺ |
| 100 | 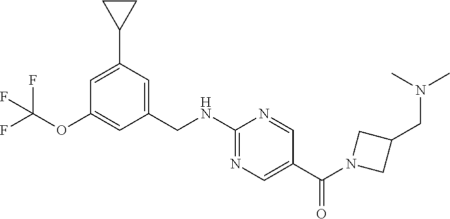 [SMILES: CN(C)CC1CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3- ((dimethylamino)methyl)azeti- din-1-yl)methanone | B25 | 450 [M + H]⁺ |
| 101 | 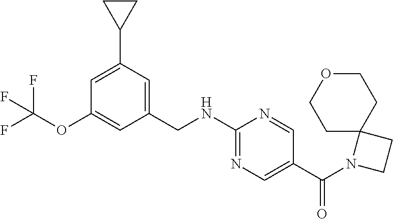 [SMILES: O=C(c1cnc(NCc2cc(OC(F)(F)F)cc(C3CC3)c2)nc1)N1CCC12CCOCC2] | (2-((3-cyclopropyl-5- (trifluoromethoxy)benzyl)ami- no)pyrimidin-5-yl)(7-oxa-1- azaspiro[3.5]nonan-1- yl)methanone | B26 | 463 [M + H]⁺ |
| 102 | 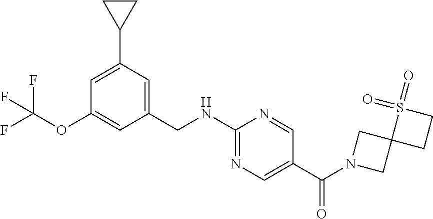 [SMILES: O=C(c1cnc(NCc2cc(OC(F)(F)F)cc(C3CC3)c2)nc1)N1CC2(CCS2(=O)=O)C1] | (2-((3-cyclopropyl-5- (trifluoromethoxy)benzyl)ami- no)pyrimidin-5-yl)(1,1- dioxido-1-thia-6- azaspiro[3.3]heptan-6- y)methanone | B27 | 483 [M + H]⁺ |
| 103 | 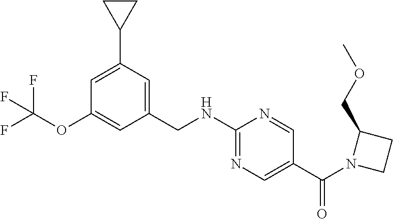 [SMILES: COC[C@H]1CCN1C(=O)c1cnc(NCc2cc(OC(F)(F)F)cc(C3CC3)c2)nc1] | (R)-(2-((3-cyclopropyl-5- (trifluoromethoxy)benzyl)ami- no)pyrimidin-5-yl)(2- (methoxymethyl)azetidin-1- yl)methanone | B28 | 437 [M + H]⁺ |
| 104 | 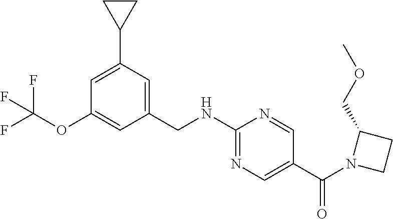 [SMILES: COC[C@@H]1CCN1C(=O)c1cnc(NCc2cc(OC(F)(F)F)cc(C3CC3)c2)nc1] | (S)-(2-((3-cyclopropyl-5- (trifluoromethoxy)benzyl)ami- no)pyrimidin-5-yl)(2- (methoxymethyl)azetidin-1- yl)methanone | B29 | 437 [M + H]⁺ |
| 105 | 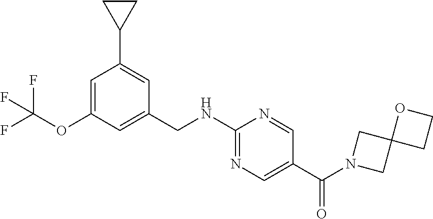 [SMILES: O=C(c1cnc(NCc2cc(OC(F)(F)F)cc(C3CC3)c2)nc1)N1CC2(CCO2)C1] | (2-((3-cyclopropyl-5- (trifluoromethoxy)benzyl)ami- no)pyrimidin-5-yl)(1-oxa-6- azaspiro[3.3]heptan-6- yl)methanone | B30 | 435 [M + H]⁺ |
| 106 | 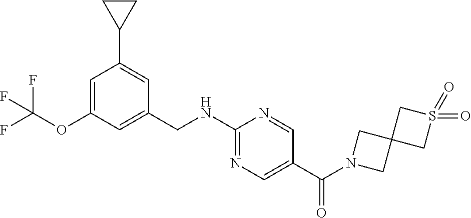 [SMILES: O=C(c1cnc(NCc2cc(OC(F)(F)F)cc(C3CC3)c2)nc1)N1CC2(C1)CS(=O)(=O)C2] | (2-((3-cyclopropyl-5- (trifluoromethoxy)benzyl)ami- no)pyrimidin-5-yl)(2,2- dioxido-2-thia-6- azaspiro[3.3]heptan-6- yl)methanone | B31 | 483 [M + H]⁺ |
| 107 | 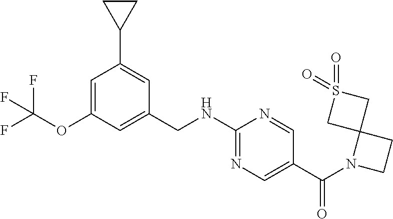 [SMILES: O=C(c1cnc(NCc2cc(OC(F)(F)F)cc(C3CC3)c2)nc1)N1CCC12CS(=O)(=O)C2] | (2-((3-cyclopropyl-5- (trifluoromethoxy)benzyl)ami- no)pyrimidin-5-yl)(6,6- dioxido-6-thia-1- azaspiro[3.3]heptan-1-yl)- methanone | B32 | 483 [M + H]⁺ |
| 108 | 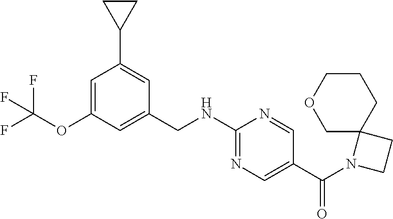 [SMILES: O=C(c1cnc(NCc2cc(OC(F)(F)F)cc(C3CC3)c2)nc1)N1CCC12CCCOC2] | (2-((3-cyclopropyl-5- (trifluoromethoxy)benzyl)ami- no)pyrimidin-5-yl)(6-oxa-1- azaspiro[3.5]nonan-1-yl)- methanone | B33 | 463 [M + H]⁺ |
| 109 | 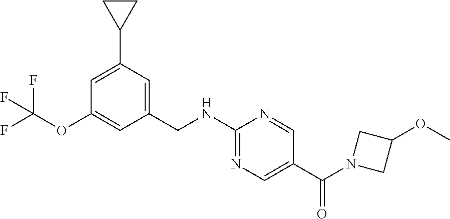 [SMILES: COC1CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3- methoxyazetidin-1-yl)- methanone | B34 | 423 [M + H]⁺ |
| 110 | 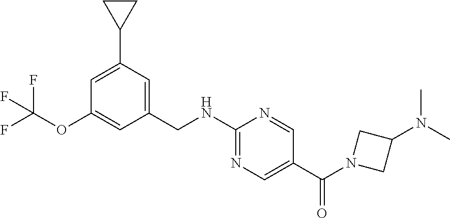 [SMILES: CN(C)C1CN(C(=O)c2cnc(NCc3cc(OC(F)(F)F)cc(C4CC4)c3)nc2)C1] | (2-((3-cyclopropyl-5- (trifluoromethoxy)benzyl)ami- no)pyrimidin-5-yl)(3- (dimethylamino)azetidin-1- yl)methanone | B35 | 436 [M + H]⁺ |
| 111 | 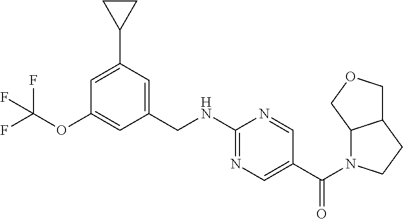 [SMILES: O=C(c1cnc(NCc2cc(OC(F)(F)F)cc(C3CC3)c2)nc1)N1CCC2COCC21] | (2-((3-cyclopropyl-5- (trifluoromethoxy)benzyl)ami- no)pyrimidin-5- yl)(hexahydro-1H-furo[3,4- b]pyrrol-1-yl)methanone | B36 | 449 [M + H]⁺ |
| 112 | 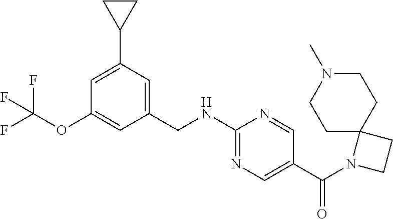 [SMILES: CN1CCC2(CC1)CCN2C(=O)c1cnc(NCc2cc(OC(F)(F)F)cc(C3CC3)c2)nc1] | (2-((3-cyclopropyl-5- (trifluoromethoxy)benzyl)ami- no)pyrimidin-5-yl)(7- methyl-1,7- diazaspiro[3.5]nonan-1- yl)methanone | B37 | 476 [M + H]⁺ |
| 113 | 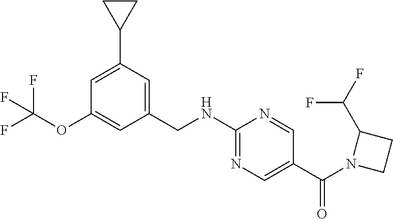 [SMILES: O=C(c1cnc(NCc2cc(OC(F)(F)F)cc(C3CC3)c2)nc1)N1CCC1C(F)F] | (2-((3-cyclopropyl-5- (trifluoromethoxy)benzyl)ami- no)pyrimidin-5-yl)(2- (difluoromethyl)azetidin-1- yl)methanone | B38 | 443 [M + H]⁺ |
| 114 | 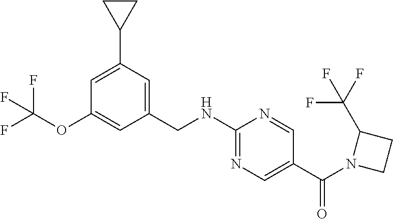 [SMILES: O=C(c1cnc(NCc2cc(OC(F)(F)F)cc(C3CC3)c2)nc1)N1CCC1C(F)(F)F] | (2-((3-cyclopropyl-5- (trifluoromethoxy)benzyl)ami- no)pyrimidin-5-yl)(2- (trifluoromethyl)azetidin-1- yl)methanone | B39 | 461 [M + H]⁺ |
| 115 | 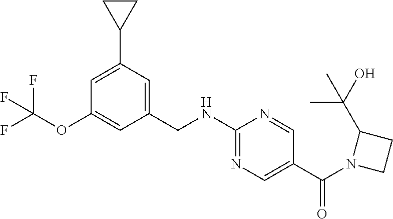 [SMILES: CC(C)(O)C1CCN1C(=O)c1cnc(NCc2cc(OC(F)(F)F)cc(C3CC3)c2)nc1] | (2-((3-cyclopropyl-5- (trifluoromethoxy)benzyl)ami- no)pyrimidin-5-yl)(2-(2- hydroxypropan-2-yl)azetidin- 1-yl)methanone | B43 | 451 [M + H]⁺ |

### Example 116 and Example 117. (2-((3-Cyclopropyl-5-(trifluoromethoxy)-benzyl)amino)pyrimidin-5-yl)(6-oxa-1-azaspiro[3.4]octan-1-yl) methanone, R and S Enantiomers

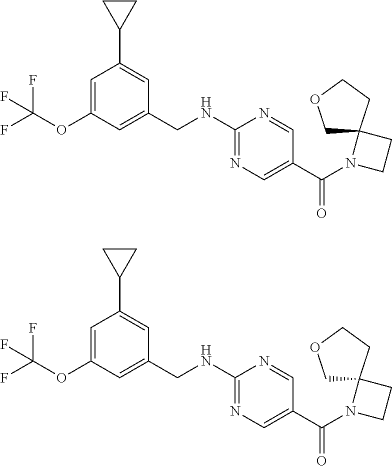 [SMILES: O=C(c1cnc(NCc2cc(OC(F)(F)F)cc(C3CC3)c2)nc1)N1CC[C@@]12CCOC2 ; O=C(c1cnc(NCc2cc(OC(F)(F)F)cc(C3CC3)c2)nc1)N1CC[C@]12CCOC2]

Racemic product of example 97 was resolved by chiral SFC (stationary phase: Lux Cellulose-2 4.6×250 mm, 5 m; mobile phase: 5% to 55% methanol+0.1% formic acid, 100 Bar of CO₂; flow rate: 10 mL/min) into its two enantio-enriched (>99% ee) antipodes. First eluting enantiomer, example 116; RT: 3.49 min, MS (ESI): m/z=449 [M+H]⁺. Second eluting enantiomer, example 117; RT: 4.05 min, MS (ESI): m/z=449 [M+H]⁺.

### Example 118, Example 119 and Example 120 (2-((3-Cyclopropyl-5-(trifluoromethoxy)-benzyl)amino)pyrimidin-5-yl)(2,4-dimethylazetidin-1-yl)methanone, (2S, 4S) and (2R, 4R) trans Diastereomers and (2R, 4S) cis Diastereomer

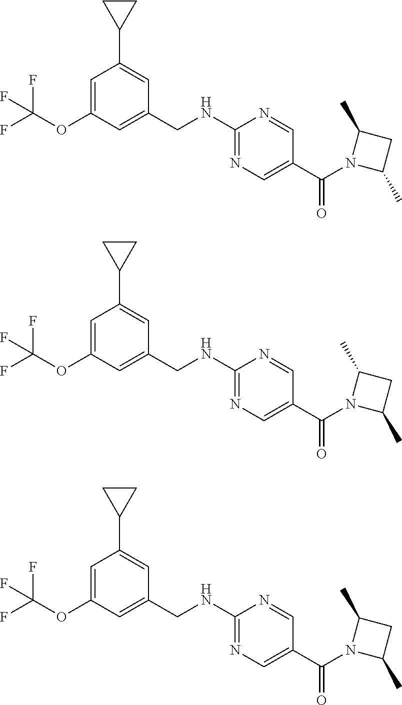 [SMILES: C[C@H]1C[C@H](C)N1C(=O)c1cnc(NCc2cc(OC(F)(F)F)cc(C3CC3)c2)nc1 ; C[C@@H]1C[C@@H](C)N1C(=O)c1cnc(NCc2cc(OC(F)(F)F)cc(C3CC3)c2)nc1 ; C[C@@H]1C[C@H](C)N1C(=O)c1cnc(NCc2cc(OC(F)(F)F)cc(C3CC3)c2)nc1]

Diastereomeric product mixture of example 93 was resolved by chiral SFC (stationary phase: Lux Cellulose-2 10×250 mm, 5 m; mobile phase: 40% methanol, 100 Bar of CO₂; flow rate: 10 mL/min) into its three diastereo-enriched (>99% de) components. First eluting trans diastereomer, example 118; RT: 4.20 min, MS (ESI): m/z=421 [M+H]⁺. Second eluting trans diastereomer, example 119; RT: 5.26 min, MS (ESI): m/z=421 [M+H]⁺. Third eluting cis diastereomer, example 120; RT: 8.18 min, MS (ESI): m/z=421 [M+H]⁺.

### Example 121 and Example 122. (2-((3-Cyclopropyl-5-(trifluoromethoxy)benzyl)amino)-pyrimidin-5-yl)(6-oxa-1-azaspiro[3.5]nonan-1-yl)methanone, R and S Enantiomers

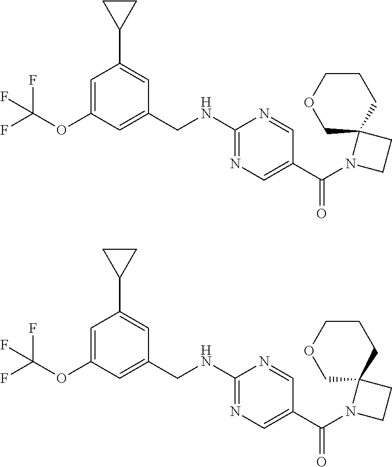 [SMILES: O=C(c1cnc(NCc2cc(OC(F)(F)F)cc(C3CC3)c2)nc1)N1CC[C@@]12CCCOC2 ; O=C(c1cnc(NCc2cc(OC(F)(F)F)cc(C3CC3)c2)nc1)N1CC[C@]12CCCOC2]

Racemic product of example 108 was resolved by chiral SFC (stationary phase: ChiralPak AD 10×250 mm, 5 m; mobile phase: 55% methanol, 100 Bar of CO₂; flow rate: 10 mL/min) into its two enantio-enriched (>99% ee) antipodes. First eluting enantiomer, example 121; RT: 4.88 min, MS (ESI): m/z=463 [M+H]⁺. Second eluting enantiomer, example 122; RT: 9.27 min, MS (ESI): m/z=463 [M+H]⁺.

### Example 123. (2-((3-Ethyl-5-(trifluoromethoxy)benzyl)amino)pyrimidin-5-yl)(6-oxa-1-azaspiro[3.3]heptan-1-yl)methanone

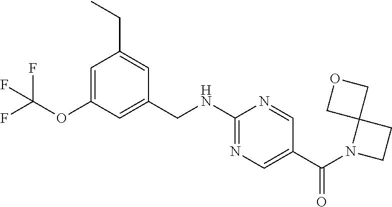 [SMILES: CCc1cc(CNc2ncc(C(=O)N3CCC34COC4)cn2)cc(OC(F)(F)F)c1]

The title compound was prepared in analogy to Example 79 using ethylboronic acid in Step 2 and 6-oxa-1-azaspiro[3.3]heptane hemioxalate (intermediate B1) in Step 4. MS (ESI): m/z=423 [M+H]⁺

### Example 124. (2-((5-Methyl-2,3-dihydro-1H-inden-2-yl)amino)pyrimidin-5-yl)(6-oxa-1-azaspiro[3.3]heptan-1-yl)methanone

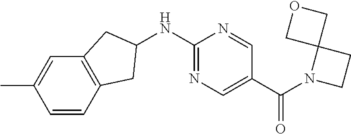 [SMILES: Cc1ccc2c(c1)CC(Nc1ncc(C(=O)N3CCC34COC4)cn1)C2]

The title compound was prepared in analogy to Example 79 using 5-bromo-2,3-dihydro-1H-inden-2-amine hydrobromide (intermediate A21) in Step 1, methylboronic acid in step 2 and 6-oxa-1-azaspiro[3.3]heptane hemioxalate (intermediate B1) in Step 4. MS (ESI): m/z=351 [M+H]⁺

### Example 125. (2-((5-Cyclopropyl-2,3-dihydro-1H-inden-2-yl)amino)pyrimidin-5-yl)(6-oxa-1-azaspiro[3.3]heptan-1-yl)methanone

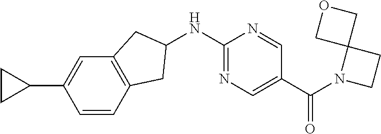 [SMILES: O=C(c1cnc(NC2Cc3ccc(C4CC4)cc3C2)nc1)N1CCC12COC2]

The title compound was prepared in analogy to Example 79 using 5-bromo-2,3-dihydro-1H-inden-2-amine hydrobromide (intermediate A21) in Step 1 and 6-oxa-1-azaspiro[3.3]heptane hemioxalate (intermediate B1) in Step 4. MS (ESI): m/z=377 [M+H]⁺

### Example 126. (2-((5-Ethyl-2,3-dihydro-1H-inden-2-yl)amino)pyrimidin-5-yl) (6-oxa-1-azaspiro[3.3]heptan-1-yl)methanone

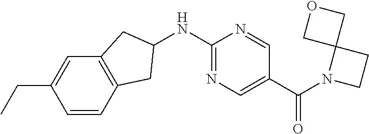 [SMILES: CCc1ccc2c(c1)CC(Nc1ncc(C(=O)N3CCC34COC4)cn1)C2]

The title compound was prepared in analogy to Example 79 using 5-bromo-2,3-dihydro-1H-inden-2-amine hydrobromide (intermediate A21) in Step 1, ethylboronic acid in step 2 and 6-oxa-1-azaspiro[3.3]heptane hemioxalate (intermediate B1) in Step 4. MS (ESI): m/z=365 [M+H]⁺

### Example 127. (2-((5,6-Dibromo-2,3-dihydro-1H-inden-2-yl)amino)pyrimidin-5-yl)(6-oxa-1-azaspiro[3.3]heptan-1-yl)methanone

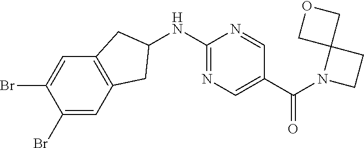 [SMILES: O=C(c1cnc(NC2Cc3cc(Br)c(Br)cc3C2)nc1)N1CCC12COC2]

### Step 1: Ethyl 2-((5,6-dibromo-2,3-dihydro-1H-inden-2-yl)amino)pyrimidine-5-carboxylate

A byproduct isolated from step 1 of example 62 resulting from the presence 5,6-dibromo-2,3-dihydro-1H-inden-2-amine as a contaminant in commercially acquired 5-bromo-2,3-dihydro-1H-inden-2-amine hydrobromide (intermediate A21) was identified as ethyl 2-((5,6-dibromo-2,3-dihydro-1H-inden-2-yl)amino)pyrimidine-5-carboxylate.

### Step 2: 2-((5,6-Dibromo-2,3-dihydro-1H-inden-2-yl)amino)pyrimidine-5-carboxylic acid

Ethyl 2-((5,6-dibromo-2,3-dihydro-1H-inden-2-yl)amino)pyrimidine-5-carboxylate from the previous step was converted to the corresponding carboxylic acid using a method analogous to step 3 of example 79.

### Step 3: (2-((5,6-Dibromo-2,3-dihydro-1H-inden-2-yl)amino)pyrimidin-5-yl)(6-oxa-1-azaspiro[3.3]heptan-1-yl)methanone

The title compound was prepared in analogy to Step 4 of Example 79 using 2-((5,6-dibromo-2,3-dihydro-1H-inden-2-yl)amino)pyrimidine-5-carboxylic acid and 6-oxa-1-azaspiro[3.3]heptane hemioxalate (intermediate B1). MS (ESI): m/z=495 [M+H]⁺

### Example 128. (2-((5,6-Dimethyl-2,3-dihydro-1H-inden-2-yl)amino)pyrimidin-5-yl)(6-oxa-1-azaspiro[3.3]heptan-1-yl)methanone

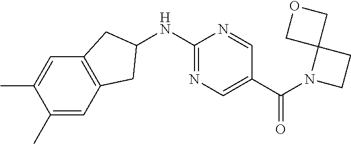 [SMILES: Cc1cc2c(cc1C)CC(Nc1ncc(C(=O)N3CCC34COC4)cn1)C2]

The title compound was prepared in analogy to Example 79 (steps 2 through 4) using ethyl 2-((5,6-dibromo-2,3-dihydro-1H-inden-2-yl)amino)pyrimidine-5-carboxylate (prepared in Step 1 of Example 127), 2 eq. of methylboronic acid in Step 2 and 6-oxa-1-azaspiro[3.3]heptane hemioxalate (intermediate B1) in Step 4. MS (ESI): m/z=365 [M+H]⁺

### Example 129. (R)-(2-((5-Chloro-2,3-dihydro-1H-inden-2-yl)amino)pyrimidin-5-yl)(6-oxa-1-azaspiro[3.3]heptan-1-yl)methanone

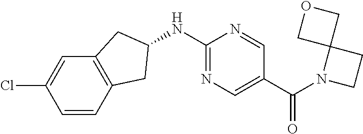 [SMILES: O=C(c1cnc(N[C@@H]2Cc3ccc(Cl)cc3C2)nc1)N1CCC12COC2]

### Step 1: (R)-Ethyl 2-((5-bromo-2,3-dihydro-1H-inden-2-yl)amino)pyrimidine-5-carboxylate

The title compound was prepared in analogy to Step 1 of Example 79 using (R)-5-bromo-2,3-dihydro-1H-inden-2-amine hydrochloride (intermediate A26).

### Step 2: (R)-Ethyl 2-((5-chloro-2,3-dihydro-1H-inden-2-yl)amino)pyrimidine-5-carboxylate

To a solution of (R)-ethyl 2-((5-bromo-2,3-dihydro-1H-inden-2-yl)amino)pyrimidine-5-carboxylate (0.51 g, 1.4 mmol) in DMF (10 mL) was added CuCl (0.28 mL, 2.8 mmol, 2 eq.) and the mixture was stirred at 150° C. for 18 h. The mixture was partitioned between EtOAc and aq. 1N HCl. The organic layer was washed with brine, dried over MgSO₄, filtered and concentrated to give (R)-ethyl 2-((5-chloro-2,3-dihydro-1H-inden-2-yl)amino)pyrimidine-5-carboxylate (0.440 g, 98% yield).

### Step 3: (R)-2-((5-Chloro-2,3-dihydro-1H-inden-2-yl)amino)pyrimidine-5-carboxylic acid

The title compound was prepared in analogy to Step 3 of Example 79 using (R)-ethyl 2-((5-chloro-2,3-dihydro-1H-inden-2-yl)amino)pyrimidine-5-carboxylate to give (R)-2-((5-chloro-2,3-dihydro-1H-inden-2-yl)amino)pyrimidine-5-carboxylic acid.

### Step 4: (R)-(2-((5-Chloro-2,3-dihydro-1H-inden-2-yl)amino)pyrimidin-5-yl)(6-oxa-1-azaspiro[3.3]heptan-1-yl)methanone

The title compound was prepared in analogy to Step 4 of Example 79 using (R)-2-((5-chloro-2,3-dihydro-1H-inden-2-yl)amino)pyrimidine-5-carboxylic acid and 6-oxa-1-azaspiro[3.3]heptane hemioxalate (intermediate B1). MS (ESI): m/z=371 [M+H]⁺

### Example 130. (S)-(2-((5-Chloro-2,3-dihydro-1H-inden-2-yl)amino)pyrimidin-5-yl)(6-oxa-1-azaspiro[3.3]heptan-1-yl)methanone

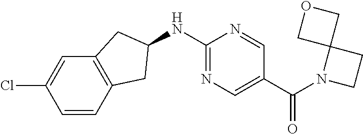 [SMILES: O=C(c1cnc(N[C@H]2Cc3ccc(Cl)cc3C2)nc1)N1CCC12COC2]

The title compound was prepared in analogy to Example 129 using (S)-5-bromo-2,3-dihydro-1H-inden-2-amine hydrochloride (intermediate A25) in step. MS (ESI): m/z=371 [M+H]⁺

### Example 131. (2-(((R)-5-Chloro-2,3-dihydro-1H-inden-2-yl)amino)pyrimidin-5-yl)(2-(2-hydroxypropan-2-yl)azetidin-1-yl)methanone

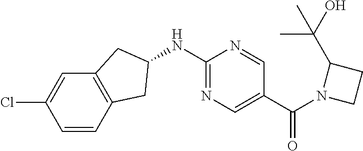 [SMILES: CC(C)(O)C1CCN1C(=O)c1cnc(N[C@@H]2Cc3ccc(Cl)cc3C2)nc1]

The title compound was prepared in analogy to Example 129 using 2-(azetidin-2-yl)propan-2-ol 2,2,2-trifluoroacetate (intermediate B43) in Step 4. MS (ESI): m/z=387 [M+H]⁺

### Example 132. (2-(((R)-5-Chloro-2,3-dihydro-1H-inden-2-yl)amino)pyrimidin-5-yl)(6-oxa-1-azaspiro[3.4]octan-1-yl)methanone

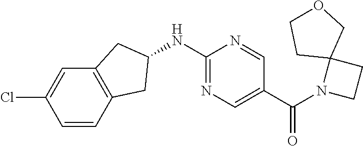 [SMILES: O=C(c1cnc(N[C@@H]2Cc3ccc(Cl)cc3C2)nc1)N1CCC12CCOC2]

The title compound was prepared in analogy to Example 129 using 6-oxa-1-azaspiro[3.4]octane hemioxalate (intermediate B22) in Step 4. MS (ESI): m/z=385 [M+H]⁺

### Example 133. (2-(((R)-5-Chloro-2,3-dihydro-1H-inden-2-yl)amino)pyrimidin-5-yl)(2-methylazetidin-1-yl)methanone

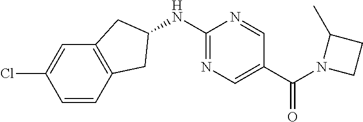 [SMILES: CC1CCN1C(=O)c1cnc(N[C@@H]2Cc3ccc(Cl)cc3C2)nc1]

The title compound was prepared in analogy to Example 129 using 2-methylazetidine hydrochloride (intermediate B3) in Step 4. MS (ESI): m/z=343 [M+H]⁺

### Example 134. (R)-(2-((5-Chloro-2,3-dihydro-1H-inden-2-yl)amino)pyrimidin-5-yl)(6-hydroxy-6-methyl-1-azaspiro[3.3]heptan-1-yl)methanone

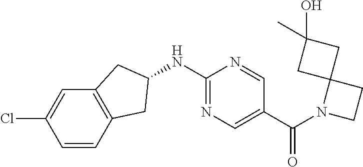 [SMILES: CC1(O)CC2(CCN2C(=O)c2cnc(N[C@@H]3Cc4ccc(Cl)cc4C3)nc2)C1]

The title compound was prepared in analogy to Example 129 using 6-methyl-1-azaspiro[3.3]heptan-6-ol 2,2,2-trifluoroacetate (intermediate B46) in Step 4. MS (ESI): m/z=399 [M+H]⁺

### Example 135. 2-(((R)-5-Chloro-2,3-dihydro-1H-inden-2-yl)amino)pyrimidin-5-yl) ((S)-2-(2-hydroxypropan-2-yl)azetidin-1-yl)methanone

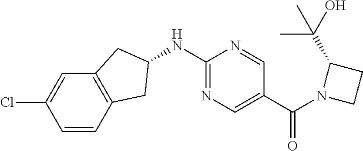 [SMILES: CC(C)(O)[C@@H]1CCN1C(=O)c1cnc(N[C@@H]2Cc3ccc(Cl)cc3C2)nc1]

The title compound was prepared in analogy to Example 129 using (S)-2-(azetidin-2-yl)propan-2-ol 2,2,2-trifluoroacetate (intermediate B44) in Step 4. MS (ESI): m/z=387 [M+H]⁺

### Example 136. (2-(((R)-5-Chloro-2,3-dihydro-1H-inden-2-yl)amino)pyrimidin-5-yl) ((R)-2-(2-hydroxypropan-2-yl)azetidin-1-yl)methanone

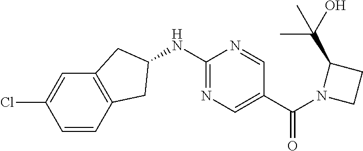 [SMILES: CC(C)(O)[C@H]1CCN1C(=O)c1cnc(N[C@@H]2Cc3ccc(Cl)cc3C2)nc1]

The title compound was prepared in analogy to Example 129 using (R)-2-(azetidin-2-yl)propan-2-ol 2,2,2-trifluoroacetate (intermediate B45) in Step 4. MS (ESI): m/z=387 [M+H]⁺

### Example 137. (2-((3-(Methylsulfonyl)-5-(trifluoromethoxy)benzyl)amino) pyrimidin-5-yl)(6-oxa-1-azaspiro[3.3]heptan-1-yl)methanone

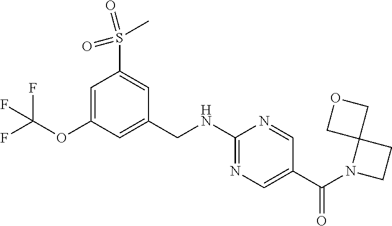 [SMILES: CS(=O)(=O)c1cc(CNc2ncc(C(=O)N3CCC34COC4)cn2)cc(OC(F)(F)F)c1]

### Step 1: Ethyl 2-((3-(methylsulfonyl)-5-(trifluoromethoxy)benzyl)amino)pyrimidine-5-carboxylate

To a solution of ethyl 2-((3-bromo-5-(trifluoromethoxy)benzyl)amino)pyrimidine-5-carboxylate (0.21 g, 0.50 mmol, 1.0 eq., prepared in step 1 of example 79) in DMSO (2 mL) was added copper(I) trifluoromethanesulfonate benzene complex (2:1) (0.013 g, 0.050 mmol, 10 mol %), sodium methanesulfinate (0.18 g, 1.5 mmol, 3.0 eq.), and N,N-dimethylethylenediamine (0.11 mL, 1.0 mmol, 2.0 eq.). The mixture was heated to 130° C. for 18 h then cooled to RT and partitioned between EtOAc and water. The organic layer was separated, washed with brine, dried over MgSO₄, filtered and concentrated. The residue was purified on SiO₂ to give ethyl 2-((3-(methylsulfonyl)-5-(trifluoromethoxy)benzyl)amino)-pyrimidine-5-carboxylate (0.060 g, 29% yield).

### Step 2: 2-((3-(Methylsulfonyl)-5-(trifluoromethoxy)benzyl)amino)pyrimidine-5-carboxylic acid

The title compound was prepared in analogy to Step 3 of Example 79.

### Step 3: (2-((3-(Methylsulfonyl)-5-(trifluoromethoxy)benzyl)amino)pyrimidin-5-yl)(6-oxa-1-azaspiro[3.3]heptan-1-yl)methanone

The title compound was prepared in analogy to Step 4 of Example 79 using 2-((3-(methylsulfonyl)-5-(trifluoromethoxy)benzyl)amino)pyrimidine-5-carboxylic acid and 6-oxa-1-azaspiro[3.3]heptane hemioxalate (intermediate B1). MS (ESI): m/z=473 [M+H]⁺

### Example 138. (2-((5-(Methylsulfonyl)-2,3-dihydro-1H-inden-2-yl)amino) pyrimidin-5-yl)(6-oxa-1-azaspiro[3.3]heptan-1-yl)methanone

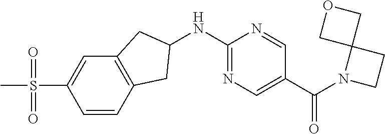 [SMILES: CS(=O)(=O)c1ccc2c(c1)CC(Nc1ncc(C(=O)N3CCC34COC4)cn1)C2]

The title compound was prepared in analogy to Example 137 using ethyl 2-((5-bromo-2,3-dihydro-1H-inden-2-yl)amino)pyrimidine-5-carboxylate (prepared in Step 1 of Example 43) in Step 1. MS (ESI): m/z=415 [M+H]⁺

### Example 139. 3-(((5-(6-Oxa-1-azaspiro[3.3]heptane-1-carbonyl)pyrimidin-2-yl)amino)methyl)-5-(trifluoromethoxy)benzonitrile

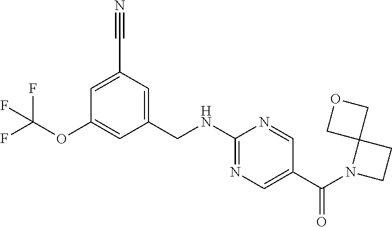 [SMILES: N#Cc1cc(CNc2ncc(C(=O)N3CCC34COC4)cn2)cc(OC(F)(F)F)c1]

### Step 1: Ethyl 2-((3cyano-5-(trifluoromethoxy)benzyl)amino)pyrimidine-5-carboxylate

To a solution of ethyl 2-((3-bromo-5-(trifluoromethoxy)benzyl)amino)pyrimidine-5-carboxylate (0.26 g, 0.61 mmol, 1.0 eq., prepared in step 1 of example 79) in DMF (3 mL) was added Zn(CN)₂ (0.11 g, 0.92 mmol, 1.5) and the mixture was purged with N₂ for 10 minutes. After adding Pd(PPh₃)₄ (0.035 g, 0.030 mmol, 0.050 eq.) the reaction vessel was sealed and heated to 100° C. for 15 h then cooled to RT and partitioned between EtOAc and water. The organic layer was separated, washed with brine, dried over MgSO₄, filtered and concentrated. The residue was purified on SiO₂ to give ethyl 2-((3cyano-5-(trifluoromethoxy)benzyl)amino)pyrimidine-5-carboxylate (0.12 g, 5500 yield).

### Step 2: 2-((3-Cyano-5-(trifluoromethoxy)benzyl)amino)pyrimidine-5-carboxylic acid

The title compound was prepared in analogy to Step 3 of Example 79.

### Step 3: 3-(((5-(6-Oxa-1-azaspiro[3.3]heptane-1-carbonyl)pyrimidin-2-yl)amino)methyl)-5-(trifluoromethoxy)benzonitrile

The title compound was prepared in analogy to Step 4 of Example 79 using 2-((3-cyano-5-(trifluoromethoxy)benzyl)amino)pyrimidine-5-carboxylic acid and 6-oxa-1-azaspiro[3.3]heptane hemioxalate (intermediate B1). MS (ESI): m/z=420 [M+H]⁺

### Example 140. 3-(((5-(6-Oxa-1-azaspiro[3.3]heptane-1-carbonyl)pyrimidin-2-yl)amino)methyl)-5-(trifluoromethoxy)benzamide

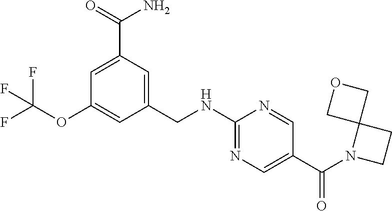 [SMILES: NC(=O)c1cc(CNc2ncc(C(=O)N3CCC34COC4)cn2)cc(OC(F)(F)F)c1]

A byproduct isolated from step 3 of example 139 was identified as 3-(((5-(6-oxa-1-azaspiro[3.3]heptane-1-carbonyl)pyrimidin-2-yl)amino)methyl)-5-(trifluoromethoxy)benzamide. MS (ESI): m/z=438 [M+H]⁺

### Example 141. (2-((3-Cyclopropyl-5-(trifluoromethoxy)benzyl)amino) pyrimidin-5-yl)(1,6-diazaspiro[3.3]heptan-1-yl)methanone

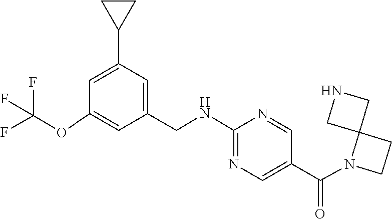 [SMILES: O=C(c1cnc(NCc2cc(OC(F)(F)F)cc(C3CC3)c2)nc1)N1CCC12CNC2]

To a solution of the product of example 92 (0.040 g, 0.074 mmol) in DCM (2 mL) was added TFA (2 mL) and the resulting solution was stirred at RT. After 1 h the mixture was partitioned between EtOAc and aq. NaHCO₃. The organic extract was washed with brine, dried over MgSO₄, filtered and concentrated to give (2-((3-cyclopropyl-5-(trifluoromethoxy)benzyl)-amino)pyrimidin-5-yl)(1,6-diazaspiro[3.3]heptan-1-yl)methanone (0.032 g, 100% yield). MS (ESI): m/z=434 [M+H]⁺

### Example 142. (2-((3-Cyclopropyl-5-(trifluoromethoxy)benzyl)amino) pyrimidin-5-yl)(6-methyl-1,6-diazaspiro[3.3]heptan-1-yl)methanone

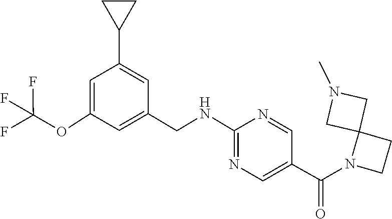 [SMILES: CN1CC2(CCN2C(=O)c2cnc(NCc3cc(OC(F)(F)F)cc(C4CC4)c3)nc2)C1]

To a solution of the product of example 141 (0.25 g, 0.46 mmol) in MeOH (5 mL) and AcOH (0.13 mL, 2.3 mmol, 5eq) was added formaldehyde (0.20 mL, 37% w/w in water, 2.3 mmol, 5 eq.) followed by Na(OAc)₃BH (0.20 g, 0.93 mmol, 2 eq.). The resulting solution was stirred at RT for 1 h and then treated with additional formaldehyde (0.20 mL, 37% w/w in water, 2.3 mmol, 5 eq.) and Na(OAc)₃BH (0.20 g, 0.93 mmol, 2 eq.). After stirring for 30 min at RT, the mixture was partitioned between EtOAc and aq. NaHCO₃. The organic layer was washed with water, brine, dried over MgSO₄, filtered, concentrated and purified on a reverse phase C₁₈ column to give (2-((3-cyclopropyl-5-(trifluoromethoxy)benzyl)amino)pyrimidin-5-yl)(6-methyl-1,6-diazaspiro[3.3]heptan-1-yl)methanone (0.12 g, 58% yield). MS (ESI): m/z=448 [M+H]⁺

### Example 143. (2-((3-Cyclopropyl-5-(trifluoromethoxy)benzyl)amino) pyrimidin-5-yl)(6-ethyl-1,6-diazaspiro[3.3]heptan-1-yl)methanone

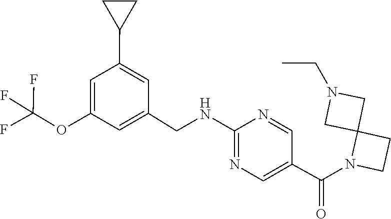 [SMILES: CCN1CC2(CCN2C(=O)c2cnc(NCc3cc(OC(F)(F)F)cc(C4CC4)c3)nc2)C1]

The title compound was prepared in analogy to Example 142 using acetaldehyde. MS (ESI): m/z=462 [M+H]⁺

### Example 144. (6-Cyclobutyl-1,6-diazaspiro[3.3]heptan-1-yl)(2-((3-cyclopropyl-5-(trifluoromethoxy)benzyl)amino)pyrimidin-5-yl)methanone

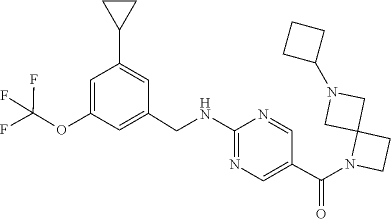 [SMILES: O=C(c1cnc(NCc2cc(OC(F)(F)F)cc(C3CC3)c2)nc1)N1CCC12CN(C1CCC1)C2]

The title compound was prepared in analogy to Example 142 using cyclobutanone. MS (ESI): m/z=488 [M+H]⁺

### Example 145. (2-((3-Cyclopropyl-5-(trifluoromethoxy)benzyl)amino)pyrimidin-5-yl) (6-(oxetan-3-yl)-1,6-diazaspiro[3.3]heptan-1-yl)methanone

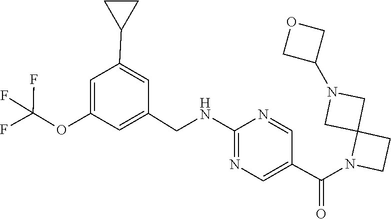 [SMILES: O=C(c1cnc(NCc2cc(OC(F)(F)F)cc(C3CC3)c2)nc1)N1CCC12CN(C1COC1)C2]

The title compound was prepared in analogy to Example 142 using 3-oxetanone. MS (ESI): m/z=490 [M+H]⁺

### Example 146. (2-((3-Cyclopropyl-5-(trifluoromethoxy)benzyl)amino)pyrimidin-5-yl) (6-cyclopropyl-1,6-diazaspiro[3.3]heptan-1-yl)methanone

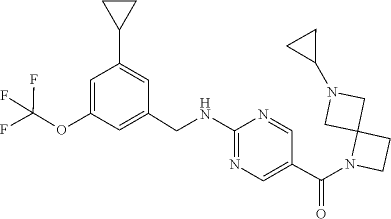 [SMILES: O=C(c1cnc(NCc2cc(OC(F)(F)F)cc(C3CC3)c2)nc1)N1CCC12CN(C1CC1)C2]

To a solution of the product of example 141 (0.10 g, 0.24 mmol) in MeOH (5 mL) and AcOH (0.068 mL, 1.2 mmol, 5 eq.) were added (1-ethoxycyclopropoxy)trimethylsilane (0.076 mL, 0.38 mmol, 1.6 eq.) and 4 Å molecular sieves. The mixture to was heated 80° C. for 3 h and then cooled to RT and treated with NaBH₃CN (0.040 g, 0.60 mmol, 2 eq.). The mixture was stirred at 40° C. for 18 h, filtered through a pad of celite and purified on a reverse phase C₁₈ column to give (2-((3-cyclopropyl-5-(trifluoromethoxy)benzyl)amino)pyrimidin-5-yl)(6-cyclopropyl-1,6-diazaspiro[3.3]heptan-1-yl)methanone (0.003 g, 3% yield). MS (ESI): m/z=474 [M+H]⁺

### Example 147. (2-((3-Cyclopropyl-5-(trifluoromethoxy)benzyl)amino)pyrimidin-5-yl) (6-isopropyl-1,6-diazaspiro[3.3]heptan-1-yl)methanone

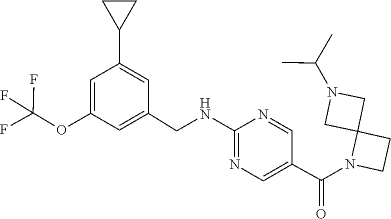 [SMILES: CC(C)N1CC2(CCN2C(=O)c2cnc(NCc3cc(OC(F)(F)F)cc(C4CC4)c3)nc2)C1]

To a solution of the product of example 141 (0.080 g, 0.19 mmol, 1.0 eq.) in DMF (2 mL) was added K₂CO₃ (0.11 g, 0.76 mmol, 4.0 eq.) and 2-bromopropane (0.087 mL, 0.92 mmol, 5.0 eq.). The mixture was stirred at 60° C. for 3.5 h then cooled to RT and partitioned between EtOAc and aq. NaHCO₃. The organic layer was washed with water, brine, dried over MgSO₄, filtered, concentrated and purified on a reverse phase C₁₈ column to give (2-((3-cyclopropyl-5-(trifluoromethoxy)benzyl)amino)pyrimidine-5-yl)(6-isopropyl-1,6-diazaspiro[3.3]heptan-1-yl)methanone) (0.01 g, 11% yield). MS (ESI): m/z=476 [M+H]⁺

### Example 148. (2-((3-Cyclopropyl-5-(trifluoromethoxy)benzyl)amino)pyrimidin-5-yl) (6-(2,2,2-trifluoroethyl)-1,6-diazaspiro[3.3]heptan-1-yl)methanone

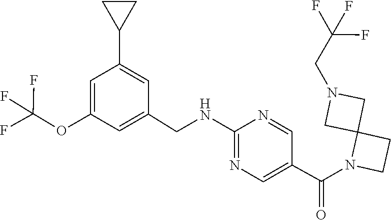 [SMILES: O=C(c1cnc(NCc2cc(OC(F)(F)F)cc(C3CC3)c2)nc1)N1CCC12CN(CC(F)(F)F)C2]

To a solution of the product of example 141 (0.10 g, 0.24 mmol) in THF (5 mL) at 0° C. was added DIPEA (0.13 mL, 0.72 mmol, 3 eq.) followed by 2,2,2-trifluoroethyl trifluoromethanesulfonate (0.077 mL, 0.53 mmol, 2.2 eq.). The resulting solution was stirred at RT for 2 h and then treated with additional 2,2,2-trifluoroethyl trifluoromethanesulfonate (0.077 mL, 0.53 mmol, 2.2 eq.) and stirred at RT for 18 h. The mixture was concentrated and the residue was purified on SiO₂ to give (2-((3-cyclopropyl-5-(trifluoromethoxy)benzyl)amino)pyrimidin-5-yl)(6-(2,2,2-trifluoroethyl)-1,6-diazaspiro[3.3]heptan-1-yl)methanone (0.060 g, 48% yield). MS (ESI): m/z=516 [M+H]⁺

### Example 149. (2-((3-Cyclopropyl-5-(trifluoromethoxy)benzyl)amino)pyrimidin-5-yl) (6-(2,2-difluoroethyl)-1,6-diazaspiro[3.3]heptan-1-yl)methanone

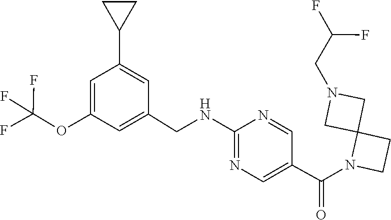 [SMILES: O=C(c1cnc(NCc2cc(OC(F)(F)F)cc(C3CC3)c2)nc1)N1CCC12CN(CC(F)F)C2]

The title compound was prepared in analogy to Example 148 using 2,2-difluoroethyl trifluoromethanesulphonate. MS (ESI): m/z=498 [M+H]⁺

### Example 150. (2-((3-Cyclopropyl-5-(trifluoromethoxy)benzyl)amino)pyrimidine-5-yl) (6-(2-(methylsulfonyl)ethyl)-1,6-diazaspiro[3.3]heptan-1-yl)methanone

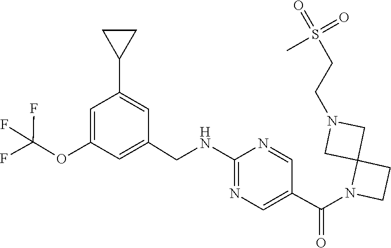 [SMILES: CS(=O)(=O)CCN1CC2(CCN2C(=O)c2cnc(NCc3cc(OC(F)(F)F)cc(C4CC4)c3)nc2)C1]

To a solution of the product of example 141 (0.07 g, 0.16 mmol, 1.0 eq.) in THF (2 mL) was added methyl vinyl sulfone (0.052 g, 0.48 mmol, 3.0 eq.) and DIPEA (0.085 mL, 0.49 mmol, 3.0 eq.). The resulting solution was stirred at RT for 18 h then concentrated and purified on a reverse phase C₁₈ column to give (2-((3-cyclopropyl-5-(trifluoromethoxy)benzyl)amino)-pyrimidine-5-yl)(6-(2-(methylsulfonyl)ethyl)-1,6-diazaspiro[3.3]heptan-1-yl)methanone) (0.041 g, 48% yield). MS (ESI): m/z=540 [M+H]⁺

### Example 151. (2-((3-Isopropyl-5-(trifluoromethoxy)benzyl)amino)pyrimidin-5-yl) (6-(methylsulfonyl)-1,6-diazaspiro[3.3]heptan-1-yl)methanone

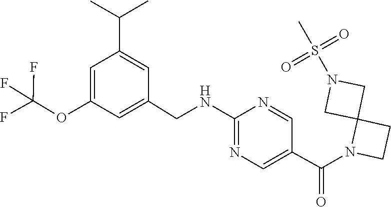 [SMILES: CC(C)c1cc(CNc2ncc(C(=O)N3CCC34CN(S(C)(=O)=O)C4)cn2)cc(OC(F)(F)F)c1]

### Step 1: (2-((3-Isopropyl-5-(trifluoromethoxy)benzyl)amino)pyrimidin-5-yl)(1,6-diazaspiro[3.3]heptan-1-yl)methanone

The title compound was prepared in analogy to Example 141 using tert-butyl 1-(2-((3-isopropyl-5-(trifluoromethoxy)benzyl)amino)pyrimidine-5-carbonyl)-1,6-diazaspiro[3.3]heptane-6-carboxylate (prepared according to Procedure B using tert-butyl 1,6-diazaspiro[3.3]heptane-6-carboxylate hemioxalate (intermediate B17) in Step 3).

### Step 2: (2-((3-Isopropyl-5-(trifluoromethoxy)benzyl)amino)pyrimidin-5-yl)(6-(methylsulfonyl)-1,6-diazaspiro[3.3]heptan-1-yl)methanone

To a solution of (2-((3-isopropyl-5-(trifluoromethoxy)benzyl)amino)pyrimidine-5-yl)(1,6-diazaspiro[3.3]heptan-1-yl)methanone) (0.082 g, 0.19 mmol, 1.0 eq.)) in DCM (2 mL) was added TEA (0.13 mL, 0.95 mmol, 5.0 eq.) and methylsulfonyl chloride (0.026 g, 0.23 mmol, 1.2 eq.). The resulting solution was stirred at RT for 18 h, then concentrated and purified on a reverse phase C₁₈ column to give (2-((3-isopropyl-5-(trifluoromethoxy)benzyl)amino)pyrimidine-5-yl)(6-methylsulfonyl)-1, 6-diazaspiro[3.3]heptan-1-yl)methanone (0.037 g, 38% yield). MS (ESI): m/z=514 [M+H]⁺

### Example 152. (2-((5-Chloro-2,3-dihydro-1H-inden-2-yl)amino)pyrimidin-5-yl)(6-(methylsulfonyl)-1,6-diazaspiro[3.3]heptan-1-yl)methanone

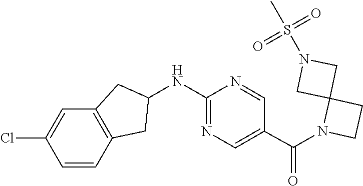 [SMILES: CS(=O)(=O)N1CC2(CCN2C(=O)c2cnc(NC3Cc4ccc(Cl)cc4C3)nc2)C1]

### Step 1: (2-((5-Chloro-2,3-dihydro-1H-inden-2-yl)amino)pyrimidin-5-yl)(1,6-diazaspiro[3.3]heptan-1-yl)methanone

The title compound was prepared in analogy to Example 141 using tert-butyl 1-(2-((5-chloro-2,3-dihydro-1H-inden-2-yl)amino)pyrimidine-5-carbonyl)-1,6-diazaspiro[3.3]-heptane-6-carboxylate (prepared according to Procedure B using 5-chloro-2,3-dihydro-1H-inden-2-amine (intermediate A18) in Step 1 and tert-butyl 1,6-diazaspiro[3.3]heptane-6-carboxylate hemioxalate (intermediate B17) in Step 3).

### Step 2: (2-((5-Chloro-2,3-dihydro-1H-inden-2-yl)amino)pyrimidin-5-yl)(6-(methylsulfonyl)-1,6-diazaspiro[3.3]heptan-1-yl)methanone

The title compound was prepared in analogy to Step 2 of Example 151 using (2-((5-chloro-2,3-dihydro-1H-inden-2-yl)amino)pyrimidin-5-yl)(1,6-diazaspiro[3.3]heptan-1-yl)-methanone. MS (ESI): m/z=448 [M+H]⁺

### Example 153. 1-(1-(2-((3-Cyclopropyl-5-(trifluoromethoxy)benzyl)amino) pyrimidine-5-carbonyl)-1,6-diazaspiro[3.3]heptan-6-yl)ethanone

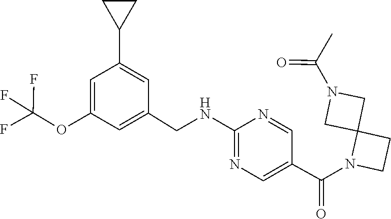 [SMILES: CC(=O)N1CC2(CCN2C(=O)c2cnc(NCc3cc(OC(F)(F)F)cc(C4CC4)c3)nc2)C1]

To a solution of the product of example 141 (0.097 g, 0.22 mmol, 1.0 eq.) in DCM (3 mL) was added DIPEA (0.047 mL, 0.27 mmol, 1.2 eq.) and acetic anhydride (0.023 mL, 0.24 mmol, 1.1 eq.). The resulting solution was stirred at RT for 15 min, concentrated and purified on a reverse phase C₁₈ column to give 1-(1-(2-((3-cyclopropyl-5-(trifluoromethoxy)benzyl)-amino)pyrimidine-5-carbonyl)-1,6-diazaspiro[3.3]heptan-6-yl)ethanone (0.048 g, 46% yield). MS (ESI): m/z=476 [M+H]⁺

### Example 154. 1-(1-(2-((5-Chloro-2,3-dihydro-1H-inden-2-yl)amino)pyrimidine-5-carbonyl)-1,6-diazaspiro[3.3]heptan-6-yl)ethanone

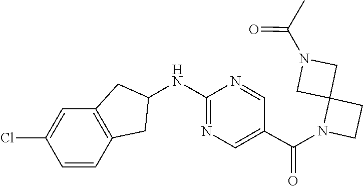 [SMILES: CC(=O)N1CC2(CCN2C(=O)c2cnc(NC3Cc4ccc(Cl)cc4C3)nc2)C1]

The title compound was prepared in analogy to Example 153 using (2-((5-chloro-2,3-dihydro-1H-inden-2-yl)amino)pyrimidin-5-yl)(1,6-diazaspiro[3.3]heptan-1-yl)methanone (prepared in Step 1 of Example 152). MS (ESI): m/z=412 [M+H]⁺

### Example 155. (2-((3-Cyclopropyl-5-(trifluoromethoxy)benzyl)amino) pyrimidin-5-yl)(6-methyl-1,6-diazaspiro[3.4]octan-1-yl)methanone

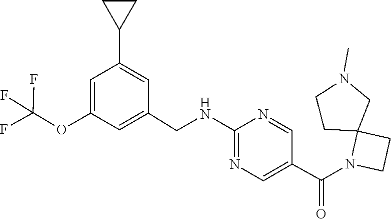 [SMILES: CN1CCC2(CCN2C(=O)c2cnc(NCc3cc(OC(F)(F)F)cc(C4CC4)c3)nc2)C1]

### Step 1: tert-Butyl 1-(2-((3-cyclopropyl-5-(trifluoromethoxy)benzyl)amino)pyrimidine-5-carbonyl)-1,6-diazaspiro[3.4]octane-6-carboxylate

The title compound was prepared in analogy to Example 79 using tert-butyl 1,6-diazaspiro[3.4]octane-6-carboxylate (intermediate B40) in Step 4.

### Step 2: (2-((3-Cyclopropyl-5-(trifluoromethoxy)benzyl)amino)pyrimidin-5-yl)(1,6-diazaspiro[3.4]octan-1-yl)methanone

The title compound was prepared in analogy to Example 141 using tert-butyl 1-(2-((3-cyclopropyl-5-(trifluoromethoxy)benzyl)amino)pyrimidine-5-carbonyl)-1,6-diazaspiro[3.4]octane-6-carboxylate.

### Step 3: (2-((3-Cyclopropyl-5-(trifluoromethoxy)benzyl)amino)pyrimidin-5-yl)(6-methyl-1,6-diazaspiro[3.4]octan-1-yl)methanone

The title compound was prepared in analogy to Example 142 using (2-((3-cyclopropyl-5-(trifluoromethoxy)benzyl)amino)pyrimidin-5-yl)(1,6-diazaspiro[3.4]octan-1-yl)methanone. MS (ESI): m/z=462 [M+H]⁺

### Example 156. (2-((3-Cyclopropyl-5-(trifluoromethoxy)benzyl)amino)pyrimidin-5-yl)(6-methyl-1,6-diazaspiro[3.5]nonan-1-yl)methanone

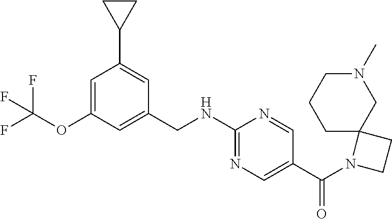 [SMILES: CN1CCCC2(CCN2C(=O)c2cnc(NCc3cc(OC(F)(F)F)cc(C4CC4)c3)nc2)C1]

The title compound was prepared in analogy to Example 155 using tert-butyl 1,6-diazaspiro[3.5]nonane-6-carboxylate (intermediate B41) in Step 1. MS (ESI): m/z=476 [M+H]⁺

### Example 157. (2-((3-Cyclopropyl-5-(trifluoromethoxy)benzyl)amino)pyrimidin-5-yl)(2-methyl-2,5-diazaspiro[3.4]octan-5-yl)methanone

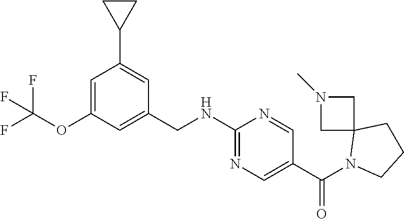 [SMILES: CN1CC2(CCCN2C(=O)c2cnc(NCc3cc(OC(F)(F)F)cc(C4CC4)c3)nc2)C1]

The title compound was prepared in analogy to Example 155 using tert-butyl 2,5-diazaspiro[3.4]octane-2-carboxylate (intermediate B42) in Step 1. MS (ESI): m/z=462 [M+H]⁺

### Example 158. (2-((3-(Azetidin-1-yl)-5-(trifluoromethoxy)benzyl)amino) pyrimidin-5-yl)(6-oxa-1-azaspiro[3.3]heptan-1-yl)methanone

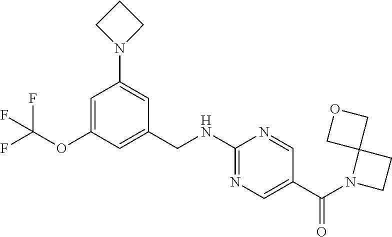 [SMILES: O=C(c1cnc(NCc2cc(OC(F)(F)F)cc(N3CCC3)c2)nc1)N1CCC12COC2]

### Step 1: Ethyl 2-((3-bromo-5-(trifluoromethoxy benzyl)(tert-butoxycarbonyl)amino)pyrimidine-5-carboxylate

To a solution of ethyl 2-((3-bromo-5-(trifluoromethoxy)benzyl)amino)pyrimidine-5-carboxylate (1.00 g, 2.38 mmol, 1.0 eq., prepared step 1 of example 79) in DCM (20 mL) was added 100 mg of 4-dimethylaminopyridine and Boc₂O (2.6 g, 11.9 mmol, 5.0 eq.). The mixture was stirred at RT for 1 h, then diluted with water. The organic layer was washed with brine, dried over MgSO₄, filtered and concentrated to provide ethyl 2-((3-bromo-5-(trifluoromethoxy)benzyl)(tert-butoxycarbonyl)amino)pyrimidine-5-carboxylate (1.2 g, 98% yield) which was used without further purification.

### Step 2: 2-((3-Bromo-5-(trifluoromethoxy)benzyl)(tert-butoxycarbonyl)amino)pyrimidine-5-carboxylic acid

The title compound was prepared analogously to Step 3/Example 79 using ethyl 2-((3-bromo-5-(trifluoromethoxy)benzyl)(tert-butoxycarbonyl)amino)pyrimidine-5-carboxylate.

### Step 3: tert-Butyl (5-(6-oxa-1-azaspiro[3.3]heptane-1-carbonyl)pyrimidin-2-yl)(3-bromo-5-(trifluoromethoxy)benzyl)carbamate

The title compound was prepared in analogy to step 4 of example 79 using 2-((3-bromo-5-(trifluoromethoxy)benzyl)(tert-butoxycarbonyl)amino)pyrimidine-5-carboxylic acid and 6-oxa-1-azaspiro[3.3]heptane hemioxalate (intermediate B1).

### Step 4: (2-((3-(Azetidin-1-yl)-5-(trifluoromethoxy)benzyl)amino)pyrimidin-5-yl)(6-oxa-1-azaspiro[3.3]heptan-1-yl)methanone

To a solution of tert-butyl (5-(6-oxa-1-azaspiro[3.3]heptane-1-carbonyl)pyrimidin-2-yl(3-bromo-5-(trifluoromethoxy)benzyl)carbamate (0.13 g, 0.22 mmol, 1.0 eq.) in toluene (3 mL) was added azetidine hydrochloride (42 mg, 0.44 mmol, 2.0 eq.) and sodium tert-butoxide (0.090 g, 0.88 mmol, 4.0 eq.). The mixture was subsurface purged with nitrogen for 10 minutes, then treated with Pd(OAc)₂ (0.003 g, 0.01 mmol, 0.06 eq.) and xantphos (0.01 g, 0.02 mmol, 0.08 eq.). The reaction vessel was sealed and the mixture stirred at 100° C. for 18 h then cooled to RT and diluted with water. The organic layer was washed with brine, dried over MgSO₄, filtered and concentrated. The residue purified on a reverse phase C₁₈ column to give (2-((3-(azetidin-1-yl)-5-(trifluoromethoxy)benzyl)amino)pyrimidine-5-yl)(6-oxa-1-azaspiro[3.3]heptan-1-1-yl)methanone (0.020 g, 20% yield). MS (ESI): m/z=450 [M+H]⁺

### Example 159. (2-((3-Ethoxy-5-isopropoxybenzyl)amino)pyrimidin-5-yl) (6-oxa-1-azaspiro[3.3]heptan-1-yl)methanone

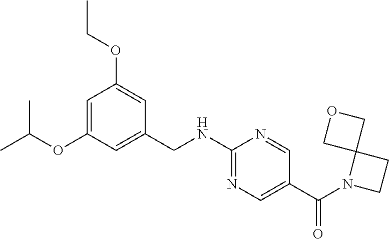 [SMILES: CCOc1cc(CNc2ncc(C(=O)N3CCC34COC4)cn2)cc(OC(C)C)c1]

### Step 1: Ethyl 2-((3-bromo-5-isopropoxybenzyl)amino)pyrimidine-5-carboxylate

The title compound was prepared in analogy to step 1 of example 79 using 3-bromo-5-isopropoxyphenyl)methanamine (intermediate A45).

### Step 2: Ethyl 2-((3-bromo-5-isopropoxybenzyl)(tert-butoxycarbonyl)amino)pyrimidine-5-carboxylate

The title compound was prepared in analogy to step 1 of example 158 using ethyl 2-((3-bromo-5-isopropoxybenzyl)amino)pyrimidine-5-carboxylate.

### Step 3: Ethyl 2-(tert-butoxycarbonyl)(3-isopropoxy-5-(4,4,5,5-tetramethyl-1,3,2-dioxaborolan-2-yl)benzyl)amino)pyrimidine-5-carboxylate

To a solution of ethyl 2-((3-bromo-5-isoproxy)benzyl)(tert-butoxycarbonyl)amino)pyrimidine-5-carboxylate (0.90 g, 1.8 mmol, 1.0 eq.) in MeCN (9 mL) was added bis(pinacolato)diboron (0.56 g, 2.2 mmol, 1.2 eq.) and KOAc (0.89 g, 9.1 mmol, 5.0 eq.). The mixture was subsurface purged with nitrogen for 10 minutes, PdCl₂(dppf) (0.27 g, 0.36 mmol, 0.2 eq.) was added to the mixture and the reaction vessel was sealed and heated to 80° C. for 18 h. After cooling to RT, the mixture was partitioned between water and EtOAc. The organic phase was separated, washed with brine, dried over MgSO₄, filtered and concentrated. The residue was purified on SiO₂ to give ethyl 2-(tert-butoxycarbonyl)(3-isopropoxy-5-(4,4,5,5-tetramethyl-1,3,2-dioxaborolan-2-yl)benzyl)amino)pyrimidine-5-carboxylate (0.89 g, 90% yield).

### Step 4: Ethyl 2-((tert-butoxycarbonyl)(3-hydroxy-5-isopropoxybenzyl)amino)pyrimidine-5-carboxylate

To a solution of ethyl 2-(tert-butoxycarbonyl)(3-isopropoxy-5-(4,4,5,5-tetramethyl-1,3,2-dioxaborolan-2-yl)benzyl)amino)pyrimidine-5-carboxylate (0.89 g, 1.6 mmol, 4.2 eq.) in THF (4.5 mL) and water (4.5 mL) was added NaBO₃ tetrahydrate (1.1 g, 6.9 mmol, 4.2 eq.). The mixture was stirred at RT for 30 min, filtered and partitioned between 10% Na₂S₂O₃ and EtOAc. The organic phase was separated, washed with brine, dried over MgSO₄, filtered and concentrated. The residue was purified on SiO₂ to give ethyl 2-((tert-butoxycarbonyl)(3-hydroxy-5-isopropoxybenzyl)amino)pyrimidine-5-carboxylate (0.56 g, 80% yield).

### Step 5: Ethyl 2-((tert-butoxycarbonyl)(3-ethoxy-5-isopropoxybenzyl)amino)pyrimidine-5-carboxylate

The title compound was prepared in analogy to step 1 of intermediate A29 using ethyl 2-((tert-butoxycarbonyl)(3-hydroxy-5-isopropoxybenzyl)amino)pyrimidine-5-carboxylate and iodoethane.

### Step 6: Ethyl 2-((3-ethoxy-5-isopropoxybenzyl)amino)pyrimidine-5-carboxylate

The title compound was prepared in analogy to step 2 of intermediate B43 using ethyl 2-((tert-butoxycarbonyl)(3-ethoxy-5-isopropoxybenzyl)amino)pyrimidine-5-carboxylate.

### Step 7: 2-((3-Ethoxy-5-isopropoxybenzyl)amino)pyrimidine-5-carboxylic acid

The title compound was prepared in analogy to step 3 of example 79 using ethyl 2-((3-ethoxy-5-isopropoxybenzyl)amino)pyrimidine-5-carboxylate.

### Step 8: (2-((3-Ethoxy-5-isopropoxybenzyl)amino)pyrimidin-5-yl)(6-oxa-1-azaspiro[3.3]heptan-1-yl)methanone

The title compound was prepared in analogy to step 4 of example 79 using 2-((3-ethoxy-5-isopropoxybenzyl)amino)pyrimidine-5-carboxylic acid and 6-oxa-1-azaspiro[3.3]heptane hemioxalate (intermediate B1). MS (ESI): m/z=413 [M+H]⁺

### Example 160. (2-((3-Ethoxy-5-isopropoxybenzyl)amino)pyrimidin-5-yl) (2-(2-hydroxypropan-2-yl)azetidin-1-yl)methanone

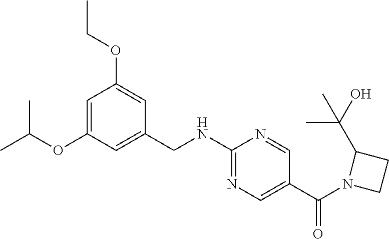 [SMILES: CCOc1cc(CNc2ncc(C(=O)N3CCC3C(C)(C)O)cn2)cc(OC(C)C)c1]

The title compound was prepared in analogy to example 159 using 2-(azetidin-2-yl)propan-2-ol 2,2,2-trifluoroacetate (intermediate B43) in step 8. MS (ESI): m/z=429 [M+H]⁺

### Example 161. (2-((5-Isopropoxy-2,3-dihydro-1H-inden-2-yl)amino) pyrimidin-5-yl)(6-oxa-1-azaspiro[3.3]heptan-1-yl)methanone

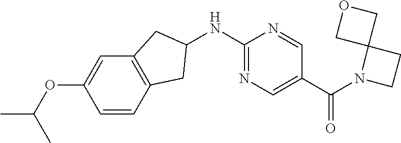 [SMILES: CC(C)Oc1ccc2c(c1)CC(Nc1ncc(C(=O)N3CCC34COC4)cn1)C2]

The title compound was prepared in analogy to example 159 using 5-bromo-2,3-dihydro-1H-inden-2-amine hydrobromide (intermediate A21) in step 1 and 2-iodopropane in step 5. MS (ESI): m/z=395 [M+H]⁺

### Example 162. (2-((5-Ethoxy-2,3-dihydro-1H-inden-2-yl)amino) pyrimidin-5-yl)(6-oxa-1-azaspiro[3.3]heptan-1-yl)methanone

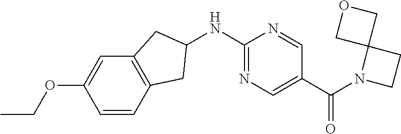 [SMILES: CCOc1ccc2c(c1)CC(Nc1ncc(C(=O)N3CCC34COC4)cn1)C2]

The title compound was prepared in analogy to Example 159 using 5-bromo-2,3-dihydro-1H-inden-2-amine hydrobromide (intermediate A21) in Step 1. MS (ESI): m/z=381 [M+H]⁺

### Example A

A compound of formula (I) can be used in a manner known per se as the active ingredient for the production of tablets of the following composition:

Per tablet

| | |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example B

A compound of formula (I) can be used in a manner known per se as the active ingredient for the production of capsules of the following composition:

Per capsule

| | |
|---|---|
| Active ingredient | 100.0 mg |
| Corn starch | 20.0 mg |
| Lactose | 95.0 mg |
| Talc | 4.5 mg |
| Magnesium stearate | 0.5 mg |
| | 220.0 mg |

In another embodiment, any one of the above described embodiments can be used alone or in combination with any one or more of the above described embodiments.

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

1. A compound having the formula (I): 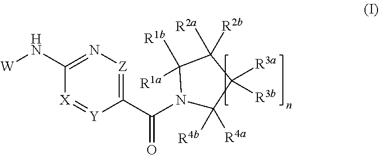
or a pharmaceutically acceptable salt, solvate or salt of the solvate, wherein
W is selected from 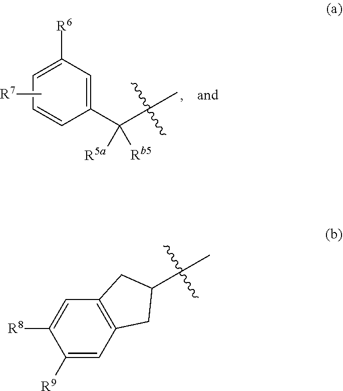
X, Y and Z are each independently selected from N and CH;
n is 0, 1 or 2;
R^{1a} and R^{1b}, R^{4a} and R^{4b} are each independently selected from
(a) hydrogen,
(b) C₁₋₆alkyl optionally substituted with OR^{a}, and
(c) C₁₋₆haloalkyl;
R^{2a}, R^{2b} R^{3a}, and R^{3b} are each independently selected from
(a) hydrogen,
(b) C₁₋₆alkyl optionally substituted with OR^{a} or NR^{b}R^{c},
(c) C₁₋₆haloalkyl,
(d) halogen,
(e) OR^{a},
(f) NR^{b}R^{c}, and
(g) S(O)ₘC₁₋₆alkyl; or
R^{1a}+R^{1b}, or R^{2a}+R^{2b}, or R^{3a}+R^{3b}, together with the carbon atom to which each pair is attached form a
(a) 4- to 6-membered carbocycle optionally substituted with one to three groups independently selected from C₁₋₄alkyl, and OH; or
(b) 4- to 6-membered heterocycle having 1 heteroatom selected from O, S(O)ₘ, and N—R^{b}; or
R^{1a}+R^{2a}, or R^{2a}+R^{3a} and the carbon atoms to which each pair is attached together form a
(a) 5- to 6-membered heterocycle having 1 heteroatom selected from O, S(O)ₘ, and N—R^{b}; or
(b) 5- to 6-membered heteroaryl;
R^{5a} and R^{5b} are each independently selected from
(a) hydrogen, and
(b) C₁₋₆alkyl,
R⁶ is selected from
(a) C₁₋₆alkyl,
(b) C₁₋₆haloalkyl,
(c) (CH₂)ₚC₃₋₆cycloalkyl,
(d) OR^{a},
(e) NR^{b}R^{c},
(f) halogen
(g) SF₅,
(h) CN,
(i) S(O)ₘC₁₋₆alkyl, and
(j) C(O)NR^{b}R^{c},
R⁷, R⁸ and R⁹ are each independently selected from
(a) hydrogen,
(b) C₁₋₆alkyl,
(c) C₁₋₆haloalkyl,
(d) (CH₂)ₚC₃₋₆cycloalkyl,
(e) OR^{a},
(f) NR^{b}R^{c},
(g) halogen
(h) SF₅,
(i) CN,
(j) S(O)ₘC₁₋₆alkyl, and
(k) C(O)NR^{b}R^{c};
m is 0, 1 or 2;
p is 0, 1 or 2;
R^{a} is selected from
(a) hydrogen,
(b) C₁₋₆alkyl,
(c) C₁₋₆haloalkyl, and
(d) (CH₂)ₚC₃₋₆cycloalkyl,
R^{b} and R^{c} are independently selected from
(a) hydrogen,
(b) —C(O)C₁₋₆alkyl,
(c) —SO₂C₁₋₆alkyl,
(d) C₁₋₆alkyl optionally substituted with S(O)ₘC₁₋₆alkyl,
(e) C₁₋₆halolkyl,
(f) C₃₋₆cycloalkyl,
(g) 4- to 6-membered heterocycle, and
(h) C(O)OC₁₋₆alkyl; or
R^{b}, R^{c} and the atom to which they are attached together form a 4- to 6-membered ring optionally containing one additional heteroatom selected from O, S(O)ₘ, and NH.

2. The compound of claim 1, wherein X is N, and Y and Z are each CH.

3. The compound of claim 1, wherein n is 0 or 1.

4. The compound of claim 1, wherein R^{4a} and R^{4b} are each hydrogen.

5. The compound of claim 1, wherein
R^{1a} and R^{1b} are each independently selected from
(a) hydrogen,
(b) C₁₋₆alkyl optionally substituted with OR^{a}, and
(c) C₁₋₆haloalkyl; or
R^{1a}+R^{1b} together with the carbon atom to which the pair is attached form a
(a) 4- to 6-membered carbocycle optionally substituted with one to three groups independently selected from C₁₋₄alkyl, and OH; or
(b) 4- to 6-membered heterocycle having 1 heteroatom selected from O, S(O)ₘ, and N—R^{b}; and
R^{2a}, R^{2b}, R^{3a}, R^{3b}, R^{4a} and R^{4b} are each hydrogen.

6. The compound of claim 5, wherein one of R^{1a} and R^{1b} is hydrogen, and the other is selected from (a) hydrogen, (b) C₁₋₄alkyl optionally substituted with OH, and (c) C₁₋₄fluoroalkyl.

7. The compound of claim 5, wherein
R^{1a}+R^{1b} together with the carbon atom to which the pair is attached form a
(a) 4- to 6-membered carbocycle optionally substituted with one to three groups independently selected from C₁₋₄alkyl, and OH; or
(b) 4- to 6-membered heterocycle having 1 heteroatom selected from O, S(O)ₘ, and N—R^{b}.

8. The compound of claim 1, wherein:
one of R^{2a} and R^{2b} is hydrogen and the other is selected from
(a) hydrogen,
(b) C₁₋₆alkyl optionally substituted with OR^{a} or NR^{b}R^{c},
(c) C₁₋₆haloalkyl,
(d) halogen,
(e) OR^{a},
(f) NR^{b}R^{c}, and
(g) S(O)ₘC₁₋₆alkyl; or
R^{2a}+R^{2b} together with the carbon atom to which the pair is attached form a
(a) 4- to 6-membered carbocycle optionally substituted with one to three groups independently selected from C₁₋₄alkyl, and OH; or
(b) 4- to 6-membered heterocycle having 1 heteroatom selected from O, S(O)ₘ, and N—R^{b}; and
R^{1a}, R^{1b}, R^{3a}, R^{3b}, R^{4a} and R^{4b} are each hydrogen.

9. The compound of claim 8, wherein:
R^{2a}+R^{2b} together with the carbon atom to which the pair is attached form a 4- to 6-membered heterocycle having 1 heteroatom selected from O, S(O)ₘ, and N—R^{b}.

10. The compound of claim 1, wherein W is selected from

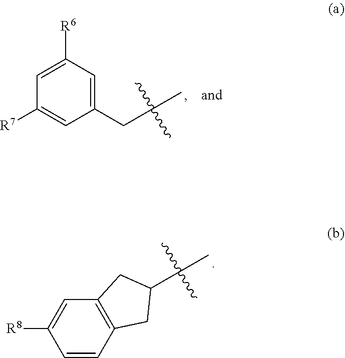

11. The compound of claim 1, having the formula (Ia) 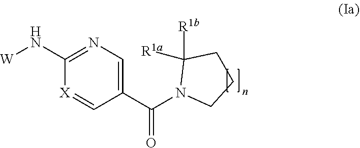
or a pharmaceutically acceptable salt, solvate or salt of the solvate thereof, wherein
W is selected from 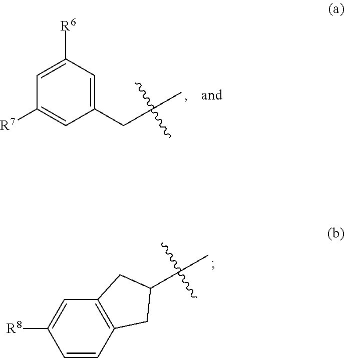
n is 0 or 1;
R⁶ is selected from
(a) C₁₋₄alkyl,
(b) C₁₋₄fluoroalkyl,
(c) (CH₂)ₚC₃₋₄cycloalkyl,
(d) OC₁₋₄alkyl,
(e) OC₁₋₄fluoroalkyl,
(f) NR^{b}R^{c},
(g) halogen,
(h) SF₅,
(i) CN, and
(j) S(O)₂C₁₋₄alkyl;
R⁷ is selected from
(a) C₁₋₄alkyl,
(b) C₁₋₄fluoroalkyl,
(c) (CH₂)ₚC₃₋₄cycloalkyl,
(d) OC₁₋₄alkyl,
(e) OC₁₋₄fluoroalkyl,
(f) O(CH₂)ₚC₃₋₄Cycloalkyl,
(g) NR^{b}R^{c},
(h) halogen, and
(i) hydrogen;
R⁸ is selected from
(a) C₁₋₄alkyl,
(b) C₃₋₄cycloalkyl,
(c) OC₁₋₄alkyl,
(d) halogen,
(e) CN, and
(f) hydrogen.

12. The compound of claim 10, wherein:
W is 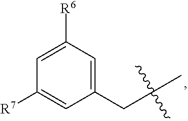
R⁶ is selected from
(a) C₁₋₄alkyl,
(b) C₁₋₂fluoroalkyl,
(c) C₃₋₄cycloalkyl,
(d) OC₁₋₄alkyl, and
(e) OC₁₋₂fluoroalkyl.
R⁷ is selected from
(a) C₁₋₄alkyl,
(b) C₁₋₂fluoroalkyl,
(c) C₃₋₄cycloalkyl,
(d) CH₂C₃₋₄cycloalkyl,
(e) OC₁₋₄alkyl,
(f) OC₁₋₂fluoroalkyl,
(g) OCH₂C₃₋₄cycloalkyl, and
(h) azetidinyl.

13. The compound of claim 11, wherein:
W is

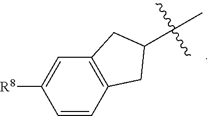

14. The compound of claim 11, wherein:
R^{1a} and R^{1b} are each independently selected from
(a) hydrogen,
(b) C₁₋₆alkyl optionally substituted with OR^{a}, and
(c) C₁₋₆haloalkyl; or
R^{1a}+R^{1b} together with the carbon atom to which the pair is attached form a
(a) 4- to 6-membered carbocycle optionally substituted with one to three groups independently selected from C₁₋₄alkyl, and OH; or
(b) 4- to 6-membered heterocycle having 1 heteroatom selected from O, S(O)ₘ, and N—R^{b}.

15. The compound of claim 11, wherein one of R^{1a} and R^{1b} is hydrogen, and the other is selected from (a) hydrogen, (b) C₁₋₄alkyl optionally substituted with OH, and (c) C₁₋₄fluoroalkyl.

16. The compound of claim 11, wherein:
R^{1a}+R^{1b} together with the carbon atom to which the pair is attached form a 4- to 6-membered heterocycle having 1 heteroatom selected from O, S(O)ₘ, and N—R^{b}; and
R^{b} is selected from
(a) hydrogen,
(b) —C(O)C₁₋₄alkyl,
(c) —SO₂C₁₋₄alkyl,
(d) C₁₋₄alkyl optionally substituted with S(O)ₘC₁₋₆alkyl,
(e) C₁₋₆halolkyl,
(f) C₃₋₄cycloalkyl,
(g) oxetanyl, and
(h) C(O)OC₁₋₄alkyl.

17. The compound of claim 11, wherein n is 0.

18. The compound of claim 1, selected from:
(2-((3-isopropyl-5-(trifluoromethoxy)benzyl)amino)pyrimidin-5-yl)(6-oxa-1-azaspiro[3.3]heptan-1-yl)methanone;
6-oxa-1-azaspiro[3.3]heptan-1-yl(2-((3-(trifluoromethoxy)benzyl)amino)pyrimidin-5-yl)-methanone;
2-oxa-6-azaspiro[3.3]heptan-6-yl(2-((3-(trifluoromethoxy)benzyl)amino)pyrimidin-5-yl)-methanone;
(2-((3-(tert-butyl)-5-(trifluoromethoxy)benzyl)amino)pyrimidin-5-yl)(6-oxa-1-azaspiro[3.3]heptan-1-yl)methanone;
(2-((3-(tert-butyl)-5-(trifluoromethoxy)benzyl)amino)pyrimidin-5-yl)(2-methylazetidin-1-yl)methanone;
(2-((3-isopropyl-5-(trifluoromethoxy)benzyl)amino)pyrimidin-5-yl)(2-methylazetidin-1-yl)-methanone;
(2-((2,3-dihydro-1H-inden-2-yl)amino)pyrimidin-5-yl)(6-oxa-1-azaspiro[3.3]heptan-1-yl)-methanone;
azetidin-1-yl(2-((2,3-dihydro-1H-inden-2-yl)amino)pyrimidin-5-yl)methanone;
(2-((3,5-bis(trifluoromethyl)benzyl)amino)pyrimidin-5-yl)(2-methylazetidin-1-yl)-methanone;
(2-((3,5-dichlorobenzyl)amino)pyrimidin-5-yl)(2-methylazetidin-1-yl)methanone;
(2-((3,5-dimethoxybenzyl)amino)pyrimidin-5-yl)(2-methylazetidin-1-yl)methanone;
(2-((3-methyl-5-(trifluoromethoxy)benzyl)amino)pyrimidin-5-yl)(2-methylazetidin-1-yl)-methanone;
6-oxa-1-azaspiro[3.3]heptan-1-yl(2-((1-(3-(trifluoromethoxy)phenyl)ethyl)amino)pyrimidin-5-yl)methanone;
3-(((5-(6-oxa-1-azaspiro[3.3]heptane-1-carbonyl)pyrimidin-2-yl)amino)methyl)-benzonitrile;
(2-((3-chlorobenzyl)amino)pyrimidin-5-yl)(6-oxa-1-azaspiro[3.3]heptan-1-yl)methanone;
(2-((3-(methylsulfonyl)benzyl)amino)pyrimidin-5-yl)(6-oxa-1-azaspiro[3.3]heptan-1-yl)-methanone;
(2-((3-(difluoromethoxy)benzyl)amino)pyrimidin-5-yl)(6-oxa-1-azaspiro[3.3]heptan-1-yl)-methanone;
(2-((3-(difluoromethyl)benzyl)amino)pyrimidin-5-yl)(6-oxa-1-azaspiro[3.3]heptan-1-yl)-methanone;
(2-((3-isopropyl-5-(pentafluorothio)benzyl)amino)pyrimidin-5-yl)(6-oxa-1-azaspiro[3.3]-heptan-1-yl)methanone;
6-oxa-1-azaspiro[3.3]heptan-1-yl(2-((3-(trifluoromethyl)benzyl)amino)pyrimidin-5-yl)-methanone;
(2-((3-isopropyl-5-(trifluoromethoxy)benzyl)amino)pyrimidin-5-yl)(pyrrolidin-1-yl)-methanone;
(2-((3-isopropyl-5-(trifluoromethoxy)benzyl)amino)pyrimidin-5-yl)(piperidin-1-yl)methanone;
(2-((3-cyclobutyl-5-(trifluoromethoxy)benzyl)amino)pyrimidin-5-yl)(6-oxa-1-azaspiro[3.3]-heptan-1-yl)methanone;
6-oxa-1-azaspiro[3.3]heptan-1-yl(2-((3-(pentafluorothio)benzyl)amino)pyrimidin-5-yl)-methanone;
(2-((3-bromo-5-(trifluoromethoxy)benzyl)amino)pyrimidin-5-yl)(6-oxa-1-azaspiro[3.3]-heptan-1-yl)methanone;
(2-((3-fluoro-5-(trifluoromethoxy)benzyl)amino)pyrimidin-5-yl)(6-oxa-1-azaspiro[3.3]-heptan-1-yl)methanone;
(2-((3-(cyclopropylmethoxy)-5-(trifluoromethoxy)benzyl)amino)pyrimidin-5-yl)(6-oxa-1-azaspiro[3.3]heptan-1-yl)methanone;
(2-((3-isopropoxy-5-(trifluoromethoxy)benzyl)amino)pyrimidin-5-yl)(6-oxa-1-azaspiro[3.3]heptan-1-yl)methanone;
(2-((3-(cyclopropylmethyl)-5-(trifluoromethoxy)benzyl)amino)pyrimidin-5-yl)(6-oxa-1-azaspiro[3.3]heptan-1-yl)methanone;
(2-((3-methoxy-5-(trifluoromethoxy)benzyl)amino)pyrimidin-5-yl)(6-oxa-1-azaspiro[3.3]-heptan-1-yl)methanone;
(2-((3-ethoxy-5-(trifluoromethoxy)benzyl)amino)pyrimidin-5-yl)(6-oxa-1-azaspiro[3.3]-heptan-1-yl)methanone;
(2-((5-methoxy-2,3-dihydro-1H-inden-2-yl)amino)pyrimidin-5-yl)(6-oxa-1-azaspiro[3.3]-heptan-1-yl)methanone;
(2-((5-chloro-2,3-dihydro-1H-inden-2-yl)amino)pyrimidin-5-yl)(6-oxa-1-azaspiro[3.3]heptan-1-yl)methanone;
2-((5-(6-oxa-1-azaspiro[3.3]heptane-1-carbonyl)pyrimidin-2-yl)amino)-2,3-dihydro-1H-indene-5-carbonitrile;
6-oxa-1-azaspiro[3.3]heptan-1-yl(2-((3-(2,2,2-trifluoroethoxy)-5-(trifluoromethoxy)-benzyl)amino)pyrimidin-5-yl)methanone;
(2-((3-cyclopropyl-5-(trifluoromethyl)benzyl)amino)pyrimidin-5-yl)(6-oxa-1-azaspiro[3.3]-heptan-1-yl)methanone;
(2-((3,5-dicyclopropylbenzyl)amino)pyrimidin-5-yl)(6-oxa-1-azaspiro[3.3]heptan-1-yl)-methanone;
(2-((3-cyclopropyl-5-isopropoxybenzyl)amino)pyrimidin-5-yl)(6-oxa-1-azaspiro[3.3]-heptan-1-yl)methanone;
(2-((3-isopropoxy-5-propylbenzyl)amino)pyrimidin-5-yl)(6-oxa-1-azaspiro[3.3]heptan-1-yl)methanone;
(2-((5-chloro-2,3-dihydro-1H-inden-2-yl)amino)pyrimidin-5-yl)(7-methyl-1,7-diazaspiro[3.5]nonan-1-yl)methanone;
(2-((5-chloro-2,3-dihydro-1H-inden-2-yl)amino)pyrimidin-5-yl)(2,2-dimethylazetidin-1-yl)-methanone;
(2-((5-fluoro-2,3-dihydro-1H-inden-2-yl)amino)pyrimidin-5-yl)(6-oxa-1-azaspiro[3.3]-heptan-1-yl)methanone;
(2-((5-bromo-2,3-dihydro-1H-inden-2-yl)amino)pyrimidin-5-yl)(6-oxa-1-azaspiro[3.3]heptan-1-yl)methanone;
(2-((5-chloro-2,3-dihydro-1H-inden-2-yl)amino)pyrimidin-5-yl)(2-methylazetidin-1-yl)-methanone;
(2-((5-chloro-2,3-dihydro-1H-inden-2-yl)amino)pyrimidin-5-yl)(2-(difluoromethyl)-azetidin-1-yl)methanone;
(2-((5-chloro-2,3-dihydro-1H-inden-2-yl)amino)pyrimidin-5-yl)(2-oxa-5-azaspiro[3.4]octan-5-yl)methanone;
(2-((5-chloro-2,3-dihydro-1H-inden-2-yl)amino)pyrimidin-5-yl)(7-oxa-1-azaspiro[3.5]nonan-1-yl)methanone;
(2-((5-chloro-2,3-dihydro-1H-inden-2-yl)amino)pyrimidin-5-yl)(6-oxa-1-azaspiro[3.5]nonan-1-yl)methanone;
(2-((5-chloro-2,3-dihydro-1H-inden-2-yl)amino)pyrimidin-5-yl)(6,6-dioxido-6-thia-1-azaspiro[3.3]heptan-1-yl)methanone;
(2-((3,5-dichlorobenzyl)amino)pyrimidin-5-yl)(6-oxa-1-azaspiro[3.3]heptan-1-yl)-methanone;
(2-((3,4-dichlorobenzyl)amino)pyrimidin-5-yl)(6-oxa-1-azaspiro[3.3]heptan-1-yl)-methanone;
(2-((3,5-diisopropoxybenzyl)amino)pyrimidin-5-yl)(6-oxa-1-azaspiro[3.3]heptan-1-yl)-methanone;
(2-((3,5-diethoxybenzyl)amino)pyrimidin-5-yl)(6-oxa-1-azaspiro[3.3]heptan-1-yl)-methanone;
(2-((2,5-dichlorobenzyl)amino)pyrimidin-5-yl)(6-oxa-1-azaspiro[3.3]heptan-1-yl)-methanone;
(2-((2,3-dichlorobenzyl)amino)pyrimidin-5-yl)(6-oxa-1-azaspiro[3.3]heptan-1-yl)-methanone;
azetidin-1-yl(2-((5-chloro-2,3-dihydro-1H-inden-2-yl)amino)pyrimidin-5-yl)methanone;
(2-((5-chloro-2,3-dihydro-1H-inden-2-yl)amino)pyrimidin-5-yl)(2-(methoxymethyl)-azetidin-1-yl)methanone;
(2-((5-chloro-2,3-dihydro-1H-inden-2-yl)amino)pyrimidin-5-yl)((S)-2-(methoxymethyl)-azetidin-1-yl)methanone;
(2-((3,5-diisopropoxybenzyl)amino)pyrimidin-5-yl)(2-(2-hydroxypropan-2-yl)azetidin-1-yl)methanone;
(2-((3-cyclopropyl-5-isopropoxybenzyl)amino)pyrimidin-5-yl)(2-(2-hydroxypropan-2-yl)-azetidin-1-yl)methanone;
(2-((5-chloro-2,3-dihydro-1H-inden-2-yl)amino)pyrimidin-5-yl)(2-(2-hydroxypropan-2-yl)-azetidin-1-yl)methanone;
(2-((5-bromo-2,3-dihydro-1H-inden-2-yl)amino)pyrimidin-5-yl)(6-oxa-1-azaspiro[3.3]-heptan-1-yl)methanone;
(S)-(2-((5-bromo-2,3-dihydro-1H-inden-2-yl)amino)pyrimidin-5-yl)(6-oxa-1-azaspiro[3.3]-heptan-1-yl)methanone;
(R)-(2-((5-bromo-2,3-dihydro-1H-inden-2-yl)amino)pyrimidin-5-yl)(6-oxa-1-azaspiro[3.3]-heptan-1-yl)methanone;
(2-((3-(azetidin-1-yl)-5-isopropoxybenzyl)amino)pyrimidin-5-yl)(6-oxa-1-azaspiro[3.3]-heptan-1-yl)methanone;
(2-(2-hydroxypropan-2-yl)azetidin-1-yl)(2-((3-isopropyl-5-(trifluoromethoxy)benzyl)-amino)pyrimidin-5-yl)methanone;
(2-((3,5-dicyclopropylbenzyl)amino)pyrimidin-5-yl)(2-(2-hydroxypropan-2-yl)azetidin-1-yl)methanone;
(2-((3-ethoxy-5-(trifluoromethoxy)benzyl)amino)pyrimidin-5-yl)(2-(2-hydroxypropan-2-yl)azetidin-1-yl)methanone;
(2-((3-cyclopropyl-5-ethoxybenzyl)amino)pyrimidin-5-yl)(6-oxa-1-azaspiro[3.3]heptan-1-yl)methanone;
(2-((3-(azetidin-1-yl)-5-(trifluoromethoxy)benzyl)amino)pyrimidin-5-yl)(2-(2-hydroxy-propan-2-yl)azetidin-1-yl)methanone;
(2-((3-isopropyl-5-(trifluoromethoxy)benzyl)amino)pyrimidin-5-yl)(2-methylazetidin-1-yl)methanone;
(R)-(2-((3-isopropyl-5-(trifluoromethoxy)benzyl)amino)pyrimidin-5-yl)(2-methylazetidin-1-yl)methanone;
(S)-(2-((3-isopropyl-5-(trifluoromethoxy)benzyl)amino)pyrimidin-5-yl)(2-methylazetidin-1-yl)methanone;
6-oxa-1-azaspiro[3.3]heptan-1-yl(5-((3-(trifluoromethoxy)benzyl)amino)pyrazin-2-yl)-methanone;
6-oxa-1-azaspiro[3.3]heptan-1-yl(6-((3-(trifluoromethoxy)benzyl)amino)pyridazin-3-yl)-methanone;
6-oxa-1-azaspiro[3.3]heptan-1-yl(6-((3-(trifluoromethoxy)benzyl)amino)pyridin-3-yl)methanone;
azetidin-1-yl(5-((3-isopropyl-5-(trifluoromethoxy)benzyl)amino)pyrazin-2-yl)methanone;
(5-((3-isopropyl-5-(trifluoromethoxy)benzyl)amino)pyrazin-2-yl)(6-oxa-1-azaspiro[3.3]heptan-1-yl)methanone;
(6-((3-isopropyl-5-(trifluoromethoxy)benzyl)amino)pyridin-3-yl)(6-oxa-1-azaspiro[3.3]heptan-1-yl)methanone;
(6-((3-cyclopropyl-5-(trifluoromethoxy)benzyl)amino)pyridin-3-yl)(6-oxa-1-azaspiro[3.3]heptan-1-yl)methanone;
(6-((5-chloro-2,3-dihydro-1H-inden-2-yl)amino)pyridin-3-yl)(6-oxa-1-azaspiro[3.3]heptan-1-yl)methanone;
(2-((3-cyclopropyl-5-(trifluoromethoxy)benzyl)amino)pyrimidin-5-yl)(1-azaspiro[3.3]heptan-1-yl)methanone;
(2-((3-cyclopropyl-5-(trifluoromethoxy)benzyl)amino)pyrimidin-5-yl)(6-oxa-1-azaspiro[3.3]heptan-1-yl)methanone;
azetidin-1-yl(2-((3-cyclopropyl-5-(trifluoromethoxy)benzyl)amino)pyrimidin-5-yl)methanone;
(2-((3-cyclopropyl-5-(trifluoromethoxy)benzyl)amino)pyrimidin-5-yl)(2-oxa-6-azaspiro[3.3]heptan-6-yl)methanone;
(2-((3-cyclopropyl-5-(trifluoromethoxy)benzyl)amino)pyrimidin-5-yl)(3-hydroxyazetidin-1-yl)methanone;
(2-((3-cyclopropyl-5-(trifluoromethoxy)benzyl)amino)pyrimidin-5-yl)(3-(2-hydroxypropan-2-yl)azetidin-1-yl)methanone;
(2-((3-cyclopropyl-5-(trifluoromethoxy)benzyl)amino)pyrimidin-5-yl)(3-fluoroazetidin-1-yl)methanone;
(2-((3-cyclopropyl-5-(trifluoromethoxy)benzyl)amino)pyrimidin-5-yl)(3,3-difluoroazetidin-1-yl)methanone;
(2-((3-cyclopropyl-5-(trifluoromethoxy)benzyl)amino)pyrimidin-5-yl)(3-hydroxy-3-methyl-azetidin-1-yl)methanone;
(2-((3-cyclopropyl-5-(trifluoromethoxy)benzyl)amino)pyrimidin-5-yl)(3-(methylsulfonyl)-azetidin-1-yl)methanone;
(2-((3-cyclopropyl-5-(trifluoromethoxy)benzyl)amino)pyrimidin-5-yl)(2,2-dimethylazetidin-1-yl)methanone;
(2-((3-cyclopropyl-5-(trifluoromethoxy)benzyl)amino)pyrimidin-5-yl)(2,3-dihydro-1H-pyrrolo[2,3-b]pyridin-1-yl)methanone;
(2-((3-cyclopropyl-5-(trifluoromethoxy)benzyl)amino)pyrimidin-5-yl)(2,3-dihydro-1H-pyrrolo[3,2-c]pyridin-1-yl)methanone;
tert-butyl 1-(2-((3-cyclopropyl-5-(trifluoromethoxy)benzyl)amino)pyrimidine-5-carbonyl)-1,6-diazaspiro[3.3]heptane-6-carboxylate;
(2-((3-cyclopropyl-5-(trifluoromethoxy)benzyl)amino)pyrimidin-5-yl)(2,4-dimethylazetidin-1-yl)methanone;
(2-((3-cyclopropyl-5-(trifluoromethoxy)benzyl)amino)pyrimidin-5-yl)(2-(methoxymethyl)-pyrrolidin-1-yl)methanone;
(S)-(2-((3-cyclopropyl-5-(trifluoromethoxy)benzyl)amino)pyrimidin-5-yl)(2-(methoxymethyl)pyrrolidin-1-yl)methanone;
(R)-(2-((3-cyclopropyl-5-(trifluoromethoxy)benzyl)amino)pyrimidin-5-yl)(2-(methoxymethyl)pyrrolidin-1-yl)methanone;
(2-((3-cyclopropyl-5-(trifluoromethoxy)benzyl)amino)pyrimidin-5-yl)(2-oxa-5-azaspiro[3.4]octan-5-yl)methanone;
(2-((3-cyclopropyl-5-(trifluoromethoxy)benzyl)amino)pyrimidin-5-yl)(6-oxa-1-azaspiro[3.4]octan-1-yl)methanone;
(2-((3-cyclopropyl-5-(trifluoromethoxy)benzyl)amino)pyrimidin-5-yl)(2,3-dihydro-1H-pyrrolo[2,3-c]pyridin-1-yl)methanone;
(2-((3-cyclopropyl-5-(trifluoromethoxy)benzyl)amino)pyrimidin-5-yl)(3-(methoxymethyl)-azetidin-1-yl)methanone;
(2-((3-cyclopropyl-5-(trifluoromethoxy)benzyl)amino)pyrimidin-5-yl)(3-((dimethylamino)-methyl)azetidin-1-yl)methanone;
(2-((3-cyclopropyl-5-(trifluoromethoxy)benzyl)amino)pyrimidin-5-yl)(7-oxa-1-azaspiro[3.5]nonan-1-yl)methanone;
(2-((3-cyclopropyl-5-(trifluoromethoxy)benzyl)amino)pyrimidin-5-yl)(1,1-dioxido-1-thia-6-azaspiro[3.3]heptan-6-yl)methanone;
(2-((3-cyclopropyl-5-(trifluoromethoxy)benzyl)amino)pyrimidin-5-yl)(2-(methoxymethyl)-azetidin-1-yl)methanone;
(R)-(2-((3-cyclopropyl-5-(trifluoromethoxy)benzyl)amino)pyrimidin-5-yl)(2-(methoxymethyl)azetidin-1-yl)methanone;
(S)-(2-((3-cyclopropyl-5-(trifluoromethoxy)benzyl)amino)pyrimidin-5-yl)(2-(methoxymethyl)azetidin-1-yl)methanone;
(2-((3-cyclopropyl-5-(trifluoromethoxy)benzyl)amino)pyrimidin-5-yl)(1-oxa-6-azaspiro[3.3]heptan-6-yl)methanone;
(2-((3-cyclopropyl-5-(trifluoromethoxy)benzyl)amino)pyrimidin-5-yl)(2,2-dioxido-2-thia-6-azaspiro[3.3]heptan-6-yl)methanone;
(2-((3-cyclopropyl-5-(trifluoromethoxy)benzyl)amino)pyrimidin-5-yl)(6,6-dioxido-6-thia-1-azaspiro[3.3]heptan-1-yl)methanone;
(2-((3-cyclopropyl-5-(trifluoromethoxy)benzyl)amino)pyrimidin-5-yl)(6-oxa-1-azaspiro[3.5]nonan-1-yl)methanone;
(2-((3-cyclopropyl-5-(trifluoromethoxy)benzyl)amino)pyrimidin-5-yl)(3-methoxyazetidin-1-yl)methanone;
(2-((3-cyclopropyl-5-(trifluoromethoxy)benzyl)amino)pyrimidin-5-yl)(3-(dimethylamino)-azetidin-1-yl)methanone;
(2-((3-cyclopropyl-5-(trifluoromethoxy)benzyl)amino)pyrimidin-5-yl)(hexahydro-1H-furo[3,4-b]pyrrol-1-yl)methanone;
(2-((3-cyclopropyl-5-(trifluoromethoxy)benzyl)amino)pyrimidin-5-yl)(7-methyl-1,7-diazaspiro[3.5]nonan-1-yl)methanone;
(2-((3-cyclopropyl-5-(trifluoromethoxy)benzyl)amino)pyrimidin-5-yl)(2-(difluoromethyl)-azetidin-1-yl)methanone;
(2-((3-cyclopropyl-5-(trifluoromethoxy)benzyl)amino)pyrimidin-5-yl)(2-(trifluoromethyl)-azetidin-1-yl)methanone;
(2-((3-cyclopropyl-5-(trifluoromethoxy)benzyl)amino)pyrimidin-5-yl)(2-(2-hydroxypropan-2-yl)azetidin-1-yl)methanone;
(2-((3-cyclopropyl-5-(trifluoromethoxy)benzyl)amino)pyrimidin-5-yl)(6-oxa-1-azaspiro[3.4]octan-1-yl)methanone;
(R)-(2-((3-cyclopropyl-5-(trifluoromethoxy)benzyl)amino)pyrimidin-5-yl)(6-oxa-1-azaspiro[3.4]octan-1-yl)methanone;
(S)-(2-((3-cyclopropyl-5-(trifluoromethoxy)benzyl)amino)pyrimidin-5-yl)(6-oxa-1-azaspiro[3.4]octan-1-yl)methanone;
(2-((3-cyclopropyl-5-(trifluoromethoxy)benzyl)amino)pyrimidin-5-yl)(2,4-dimethylazetidin-1-yl)methanone;
(2-((3-cyclopropyl-5-(trifluoromethoxy)benzyl)amino)pyrimidin-5-yl)((2R,4R)-2,4-dimethylazetidin-1-yl)methanone;
(2-((3-cyclopropyl-5-(trifluoromethoxy)benzyl)amino)pyrimidin-5-yl)((2S,4S)-2,4-dimethylazetidin-1-yl)methanone;
(2-((3-cyclopropyl-5-(trifluoromethoxy)benzyl)amino)pyrimidin-5-yl)((2R,4S)-2,4-dimethylazetidin-1-yl)methanone;
(2-((3-cyclopropyl-5-(trifluoromethoxy)benzyl)amino)pyrimidin-5-yl)(6-oxa-1-azaspiro[3.5]nonan-1-yl)methanone;
(R)-(2-((3-cyclopropyl-5-(trifluoromethoxy)benzyl)amino)pyrimidin-5-yl)(6-oxa-1-azaspiro[3.5]nonan-1-yl)methanone;
(S)-(2-((3-cyclopropyl-5-(trifluoromethoxy)benzyl)amino)pyrimidin-5-yl)(6-oxa-1-azaspiro[3.5]nonan-1-yl)methanone;
(2-((3-ethyl-5-(trifluoromethoxy)benzyl)amino)pyrimidin-5-yl)(6-oxa-1-azaspiro[3.3]heptan-1-yl)methanone;
(2-((5-methyl-2,3-dihydro-1H-inden-2-yl)amino)pyrimidin-5-yl)(6-oxa-1-azaspiro[3.3]heptan-1-yl)methanone;
(2-((5-cyclopropyl-2,3-dihydro-1H-inden-2-yl)amino)pyrimidin-5-yl)(6-oxa-1-azaspiro[3.3]heptan-1-yl)methanone;
(2-((5-ethyl-2,3-dihydro-1H-inden-2-yl)amino)pyrimidin-5-yl)(6-oxa-1-azaspiro[3.3]heptan-1-yl)methanone;
(2-((5,6-dibromo-2,3-dihydro-1H-inden-2-yl)amino)pyrimidin-5-yl)(6-oxa-1-azaspiro[3.3]-heptan-1-yl)methanone;
(2-((5,6-dimethyl-2,3-dihydro-1H-inden-2-yl)amino)pyrimidin-5-yl)(6-oxa-1-azaspiro[3.3]-heptan-1-yl)methanone;
(2-((5-chloro-2,3-dihydro-1H-inden-2-yl)amino)pyrimidin-5-yl)(6-oxa-1-azaspiro[3.3]-heptan-1-yl)methanone;
(R)-(2-((5-chloro-2,3-dihydro-1H-inden-2-yl)amino)pyrimidin-5-yl)(6-oxa-1-azaspiro[3.3]-heptan-1-yl)methanone;
(S)-(2-((5-chloro-2,3-dihydro-1H-inden-2-yl)amino)pyrimidin-5-yl)(6-oxa-1-azaspiro[3.3]-heptan-1-yl)methanone;
(2-((5-chloro-2,3-dihydro-1H-inden-2-yl)amino)pyrimidin-5-yl)(2-(2-hydroxypropan-2-yl)azetidin-1-yl)methanone;
(2-(((R)-5-chloro-2,3-dihydro-1H-inden-2-yl)amino)pyrimidin-5-yl)(2-(2-hydroxypropan-2-yl)azetidin-1-yl)methanone;
(2-((5-chloro-2,3-dihydro-1H-inden-2-yl)amino)pyrimidin-5-yl)(6-oxa-1-azaspiro[3.4]octan-1-yl)methanone;
(2-(((R)-5-chloro-2,3-dihydro-1H-inden-2-yl)amino)pyrimidin-5-yl)(6-oxa-1-azaspiro[3.4]octan-1-yl)methanone;
(2-((5-chloro-2,3-dihydro-1H-inden-2-yl)amino)pyrimidin-5-yl)(2-methylazetidin-1-yl)-methanone;
(2-(((R)-5-chloro-2,3-dihydro-1H-inden-2-yl)amino)pyrimidin-5-yl)(2-methylazetidin-1-yl)methanone;
(2-((5-chloro-2,3-dihydro-1H-inden-2-yl)amino)pyrimidin-5-yl)(6-hydroxy-6-methyl-1-azaspiro[3.3]heptan-1-yl)methanone;
(R)-(2-((5-chloro-2,3-dihydro-1H-inden-2-yl)amino)pyrimidin-5-yl)(6-hydroxy-6-methyl-1-azaspiro[3.3]heptan-1-yl)methanone;
2-((5-chloro-2,3-dihydro-1H-inden-2-yl)amino)pyrimidin-5-yl)((S)-2-(2-hydroxypropan-2-yl)azetidin-1-yl)methanone;
2-(((R)-5-chloro-2,3-dihydro-1H-inden-2-yl)amino)pyrimidin-5-yl)((S)-2-(2-hydroxypropan-2-yl)azetidin-1-yl)methanone;
(2-((5-chloro-2,3-dihydro-1H-inden-2-yl)amino)pyrimidin-5-yl)((R)-2-(2-hydroxypropan-2-yl)azetidin-1-yl)methanone;
(2-(((R)-5-chloro-2,3-dihydro-1H-inden-2-yl)amino)pyrimidin-5-yl)((R)-2-(2-hydroxypropan-2-yl)azetidin-1-yl)methanone;
(2-((3-(methylsulfonyl)-5-(trifluoromethoxy)benzyl)amino)pyrimidin-5-yl)(6-oxa-1-azaspiro[3.3]heptan-1-yl)methanone;
(2-((5-(methylsulfonyl)-2,3-dihydro-1H-inden-2-yl)amino)pyrimidin-5-yl)(6-oxa-1-azaspiro[3.3]heptan-1-yl)methanone;
3-(((5-(6-oxa-1-azaspiro[3.3]heptane-1-carbonyl)pyrimidin-2-yl)amino)methyl)-5-(trifluoromethoxy)benzonitrile;
3-(((5-(6-oxa-1-azaspiro[3.3]heptane-1-carbonyl)pyrimidin-2-yl)amino)methyl)-5-(trifluoromethoxy)benzamide;
(2-((3-cyclopropyl-5-(trifluoromethoxy)benzyl)amino)pyrimidin-5-yl)(1,6-diazaspiro[3.3]heptan-1-yl)methanone;
(2-((3-cyclopropyl-5-(trifluoromethoxy)benzyl)amino)pyrimidin-5-yl)(6-methyl-1,6-diazaspiro[3.3]heptan-1-yl)methanone;
(2-((3-cyclopropyl-5-(trifluoromethoxy)benzyl)amino)pyrimidin-5-yl)(6-ethyl-1,6-diazaspiro[3.3]heptan-1-yl)methanone;
(6-cyclobutyl-1,6-diazaspiro[3.3]heptan-1-yl)(2-((3-cyclopropyl-5-(trifluoromethoxy)-benzyl)amino)pyrimidin-5-yl)methanone;
(2-((3-cyclopropyl-5-(trifluoromethoxy)benzyl)amino)pyrimidin-5-yl)(6-(oxetan-3-yl)-1,6-diazaspiro[3.3]heptan-1-yl)methanone;
(2-((3-cyclopropyl-5-(trifluoromethoxy)benzyl)amino)pyrimidin-5-yl)(6-cyclopropyl-1,6-diazaspiro[3.3]heptan-1-yl)methanone;
(2-((3-cyclopropyl-5-(trifluoromethoxy)benzyl)amino)pyrimidin-5-yl)(6-isopropyl-1,6-diazaspiro[3.3]heptan-1-yl)methanone;
(2-((3-cyclopropyl-5-(trifluoromethoxy)benzyl)amino)pyrimidin-5-yl)(6-(2,2,2-trifluoroethyl)-1,6-diazaspiro[3.3]heptan-1-yl)methanone;
(2-((3-cyclopropyl-5-(trifluoromethoxy)benzyl)amino)pyrimidin-5-yl)(6-(2,2-difluoroethyl)-1,6-diazaspiro[3.3]heptan-1-yl)methanone;
(2-((3-cyclopropyl-5-(trifluoromethoxy)benzyl)amino)pyrimidine-5-yl)(6-(2-(methylsulfonyl)ethyl)-1,6-diazaspiro[3.3]heptan-1-yl)methanone;
(2-((3-isopropyl-5-(trifluoromethoxy)benzyl)amino)pyrimidin-5-yl)(6-(methylsulfonyl)-1,6-diazaspiro[3.3]heptan-1-yl)methanone;
(2-((5-chloro-2,3-dihydro-1H-inden-2-yl)amino)pyrimidin-5-yl)(6-(methylsulfonyl)-1,6-diazaspiro[3.3]heptan-1-yl)methanone;
1-(1-(2-((3-cyclopropyl-5-(trifluoromethoxy)benzyl)amino)pyrimidine-5-carbonyl)-1,6-diazaspiro[3.3]heptan-6-yl)ethanone;
1-(1-(2-((5-chloro-2,3-dihydro-1H-inden-2-yl)amino)pyrimidine-5-carbonyl)-1,6-diazaspiro[3.3]heptan-6-yl)ethanone;
(2-((3-cyclopropyl-5-(trifluoromethoxy)benzyl)amino)pyrimidin-5-yl)(6-methyl-1,6-diazaspiro[3.4]octan-1-yl)methanone;
(2-((3-cyclopropyl-5-(trifluoromethoxy)benzyl)amino)pyrimidin-5-yl)(6-methyl-1,6-diazaspiro[3.5]nonan-1-yl)methanone;
(2-((3-cyclopropyl-5-(trifluoromethoxy)benzyl)amino)pyrimidin-5-yl)(2-methyl-2,5-diazaspiro[3.4]octan-5-yl)methanone;
(2-((3-(azetidin-1-yl)-5-(trifluoromethoxy)benzyl)amino)pyrimidin-5-yl)(6-oxa-1-azaspiro[3.3]heptan-1-yl)methanone;
(2-((3-ethoxy-5-isopropoxybenzyl)amino)pyrimidin-5-yl)(6-oxa-1-azaspiro[3.3]heptan-1-yl)methanone;
(2-((3-ethoxy-5-isopropoxybenzyl)amino)pyrimidin-5-yl)(2-(2-hydroxypropan-2-yl)azetidin-1-yl)methanone;
(2-((5-isopropoxy-2,3-dihydro-1H-inden-2-yl)amino)pyrimidin-5-yl)(6-oxa-1-azaspiro[3.3]heptan-1-yl)methanone; and
(2-((5-ethoxy-2,3-dihydro-1H-inden-2-yl)amino)pyrimidin-5-yl)(6-oxa-1-azaspiro[3.3]heptan-1-yl)methanone,
or a pharmaceutically acceptable salt, solvate, or solvate of the salt of any of the foregoing.

19. A pharmaceutical composition, comprising a compound of claim 1, or a pharmaceutically acceptable salt thereof, and a pharmaceutically acceptable carrier.

20. A method for the treatment or prophylaxis of disease, disorder, or condition selected from the group consisting of: renal diseases, liver diseases, chronic inflammatory disorders or inflammatory diseases, respiratory diseases, vascular and cardiovascular diseases, fibrotic diseases, cancer, ocular diseases, metabolic diseases, cholestatic and other forms of chronic pruritus and acute and chronic organ transplant rejection, which comprises administering to a patient in need of such treatment a therapeutically effective amount of a compound of claim 1, or a pharmaceutically acceptable salt, solvate or salt of a solvate thereof, or the pharmaceutical composition of claim 19.

21. A method for the treatment or prophylaxis of chronic inflammatory disorders which comprises administering to a patient in need of such treatment a therapeutically effective amount of a compound of claim 1, or a pharmaceutically acceptable salt, solvate or salt of a solvate thereof, or the pharmaceutical composition of claim 19.

22. The method of claim 21, wherein the chronic inflammatory disorder is rheumatoid arthritis (RA), multiple sclerosis (MS), idiopathic pulmonary fibrosis (IPF), hepatitis or atherosclerosis.

23. The method of claim 21, wherein the chronic inflammatory disorder is multiple sclerosis.

24. A method of inhibiting ATX, comprising contacting a cell with a compound of claim 1, or a pharmaceutically acceptable salt, solvate or salt of a solvate thereof.

25. The method of claim 24, wherein the cell is a mammalian cell.

26. A method of decreasing LPA production in a cell, comprising contacting a cell with a compound of claim 1, or a pharmaceutically acceptable salt, solvate or salt of a solvate thereof.

27. The method of claim 26, wherein the cell is a mammalian cell.
